(19) United States
(12) Patent Application Publication
(10) Pub. No.: US 2025/0034599 A1
(43) Pub. Date: Jan. 30, 2025

(54) GENE DUPLICATIONS FOR CRABTREE-WARBURG-LIKE AEROBIC XYLOSE FERMENTATION

(71) Applicant: Wisconsin Alumni Research Foundation, Madison, WI (US)

(72) Inventors: Chris Todd Hittinger, Madison, WI (US); Trey Sato, Madison, WI (US); Sae-Byuk Lee, Madison, WI (US)

(73) Assignee: Wisconsin Alumni Research Foundation, Madison, WI (US)

(21) Appl. No.: 18/745,084

(22) Filed: Jun. 17, 2024

Related U.S. Application Data
(63) Continuation of application No. 18/052,410, filed on Nov. 3, 2022, now Pat. No. 12,049,660.
(60) Provisional application No. 63/275,308, filed on Nov. 3, 2021.

(51) Int. Cl. C12P 7/10; C10L 1/02; C12N 9/02; C12N 9/10; C12N 9/12; C12N 9/92

(52) U.S. Cl. CPC C12P 7/10; C10L 1/02; C12N 9/0093; C12N 9/1022; C12N 9/1205; C12N 9/92; C12Y 117/99; C12Y 202/01002; C12Y 207/01017; C12Y 503/01005; C10L 2200/0469

(57) ABSTRACT

An engineered yeast strain capable of efficient fermentation of xylose to ethanol, and methods of making and using the strain, are provided

### CROSS-REFERENCE TO RELATED APPLICATIONS

This application claims the benefit of the filing date of U.S. application No. 63/275,308, filed on Nov. 3, 2021, the disclosure of which is incorporated by reference herein.

### STATEMENT OF GOVERNMENT SUPPORT

This invention was made with government support under DE-FC02-07ER64494 and DE-SC0018409 awarded by the US Department of Energy. The government has certain rights in the invention.

### BACKGROUND

Over the past few decades, lignocellulosic biomass derived from non-edible plants, such as switchgrass, have been developed as potentially sustainable feedstocks for the production of renewable biofuels, which can replace petroleum-based fuels (Narayanaswamy et al., 2011; Sun and Jin, 2021; Williams et al., 2019). Most lignocellulosic feedstocks require thermo-chemical pretreatment and enzymatic hydrolysis to break down cellulose and hemicellulose in plant cell walls into glucose and xylose, which are the dominant hexoses and pentoses, respectively (Pauly and Keegstra, 2008). However, native Saccharomyces cerevisiae, the main fermentative microbe used by the bioethanol industry, does not consume or ferment xylose, resulting in inefficient cellulosic biofuel production whose cost is not competitive with fossil fuels (Cunha et al., 2019; Kim et al., 2013).

To overcome the economic issues impeding the commercialization of lignocellulosic-based biofuels, genetic engineering and adaptive laboratory evolution (ALE) of S. cerevisiae have been widely-used strategies for increasing the rate of xylose fermentation into ethanol, isobutanol, lactic acid and other useful biproducts. Enabling xylose utilization by S. cerevisiae has been achieved by the introduction of xylose metabolism enzymes from other fungi or bacteria. Xylose reductase and xylitol dehydrogenase (XR-XDH) (Jin et al., 2000; Johansson et al., 2001) or xylose isomerase (XI) (Brat et al., 2009; Kuyper et al., 2005) enzymes convert xylose into xylulose. Both pathways require xylulokinase (XK) to phosphorylate xylulose into xylulose-5-phosphate, which can then be converted into ethanol via the pentose phosphate pathway and glycolysis (Hahn-Hagerdal et al., 2007). Individual insertions of these pathways into S. cerevisiae have not been sufficient for xylose-to-ethanol conversion at industrially relevant rates and yields in lignocellulosic hydrolysates, prompting many to explore additional genetic improvements. See a comprehensive review on genetic discoveries that enhance xylose consumption by the XR-XDH pathway, e.g., Kwak and Jin, 2017).

For the XI pathway, several groups have employed combinations of rational engineering and ALE to uncover genetic modifications that increase xylose consumption and conversion into ethanol. Mutations in GRE3 (Lee et al., 2012; Lee et al., 2014; Sato et al., 2016; Traff et al., 2001), which encodes an aldose reductase capable of converting xylose into xylitol, an inhibitor of XI (Yamanaka, 1969); PHO13 (Bamba et al., 2016; Lee et al., 2014); and HXT7 (Reider Apel et al., 2016) were discovered by rational engineering and ALE to improve xylose consumption. Increased expression of XI by targeted or evolved integration of multiple XI copies into the genome has also been found to enhance xylose consumption (Dos Santos et al., 2016; Jeong et al., 2020). Finally, genome sequencing of strains from ALE identified synergistic interactions between loss of function mutations in HOG1, ISU1, and IRA2 genes that enhance xylose consumption by S. cerevisiae (Dos Santos et al., 2016; Sato et al., 2016). These genetic changes impacted various metabolic pathways, including xylose catabolismthe pentose phosphate pathway, glycolysis, and respiration, which together enhanced aerobic and anaerobic xylose consumption (Sato et al., 2016).

Despite the use of multiple genetic strategies, xylose fermentation by engineered S. cerevisiae strains has not attained nearly the same rate and yield as native glucose fermentation. Glucose uptake and catabolismoccur with such high flux that S. cerevisiae ferments glucose at high concentrations even in the presence of oxygen (Crabtree, 1929; Warburg et al., 1927), despite the trade off in lower ATP yield compared to aerobic respiration. This phenomenon, known as the Crabtree-Warburg Effect, may have emerged in S. cerevisiae through the evolution of intricate regulatory mechanisms, such as down regulation of genes functioning in respiration through glucose repression, and whole genome duplication (Conant and Wolfe, 2006; Lin and Li, 2011; Pfeiffer and Morley, 2014; Thompson et al., 2013). The funneling of glucose towards fermentative metabolism aerobically is thought to occur by the overflow of pyruvate from respiration into ethanol-producing fermentation, consequentially providing S. cerevisiae with a competitive advantage against ethanol-sensitive microbes in the natural environments (Kotter and Ciriacy, 1993; Pronk et al., 1996).

In contrast to glucose, several studies have determined that strains of S. cerevisiae engineered for xylose metabolism primarily respire xylose in the presence of oxygen. For example, under aerobic conditions, an engineered, xylose-fermenting strain with a mutation in ISU1 upregulated proteins involved in mitochondrial respiration when grown on xylose (Sato et al., 2016). Furthermore, treatment of this strain with Antimycin A, an inhibitor of oxidative phosphorylation, blocks aerobic growth and consumption of xylose. Others have reported that xylose-metabolizing S. cerevisiae strains recognize xylose as a non-fermentable carbon source, resulting in the up-regulation of genes related to TCA cycle, glyoxylate pathway, respiratory metabolism, and gluconeogenesis when cultivated on xylose aerobically (Jin et al., 2004; Runquist et al., 2009; Salusjarvi et al., 2008; Scalcinati et al., 2012). These results indicate that xylose flux in engineered strains may not be high enough to cause the overflow of pyruvate into ethanol aerobically, which happens naturally for glucose in yeasts demonstrating the Crabtree/Warburg Effect.

### SUMMARY

In one embodiment, an engineered Saccharomyces cerevisiae strain capable of efficient fermentation of xylose to ethanol is provided. In one embodiment, an engineered yeast may be obtained through directed evolution, where strains are identified as producing more ethanol than previously possible from a given feedstock through xylose fermentation. An engineered strain may also be prepared by introducing the genetic alterations found in the evolved strains into other strains. For instance, evolved strains are sequenced and that information is used to genetically engineer a strain that is very effective at fermenting xylose, e.g., conversion of xylose to ethanol at 55% of the theoretical maximum yield. In one embodiment, duplications of genes encoding engineered xylose metabolism enzymes, as well as TKL1, a gene encoding a transketolase in the pentose phosphate pathway, were genetic changes associated with the evolved phenotype. Reengineered duplications of genes of these enzymes, in combination with deletion mutations in HOG1, ISU1, GRE3, and IRA2, increased the rates of aerobic and anaerobic xylose fermentation. Xylose fermentation activity of an engineered strain was also demonstrated using an industrially relevant switchgrass hydrolysate (biomass processed to free sugars for fermentation). A xylose-fermenting yeast strain allows for large-scale utilization of xylose as well as glucose in processing of biomass into ethanol. Utilization of the xylose portion of biomass, e.g., from corn stover, would lead to a large increase in the overall efficiency of, for example, corn biofuel production and allow for utilization of more xylose-rich alternative feedstocks. Lignocellulose feedstocks have been cost prohibitive for the amount of ethanol recovered. Other substrates that may be employed include but are not limited to switchgrass, poplar, sorghum and sugarcane bagasse.

In one embodiment, a recombinant yeast is provided that has been genetically engineered to include one or more copies of one or more non-native genes that facilitate xylose fermentation, wherein the non-native genes include a xylulokinase gene (e.g., XYL3) or a xylose isomerase gene (e.g., XYLA); and include one or more additional copies of one or more genes, e.g., native genes, that encode a transaldolase (e.g., Tal1) or a transketolase (e.g., Tkl); and optionally include one or more of a disabling mutation in a gene encoding Cox15 polypeptide so as to exhibit reduced amounts of functional Cox15 polypeptide; a disabling mutation in a gene encoding Isu1 polypeptide so as to exhibit reduced amounts of functional Isu1 polypeptide, a disabling mutation in a gene encoding Hog1 polypeptide so as to exhibit reduced amounts of functional Hog1 polypeptide, a disabling mutation in a gene encoding Ira2 polypeptide so as to exhibit reduced amounts of functional Ira2 polypeptide, or a disabling mutation in a gene encoding Gre3 polypeptide so as to exhibit reduced amounts of functional Gre3 polypeptide, or any combination thereof. In one embodiment, the gene encoding xylulokinase and the gene encoding xylose isomerase are from different organisms. In one embodiment, the gene encoding xylulokinase and the gene encoding xylose isomerase are from the same organism. In one embodiment, the gene encoding xylulokinase is from a different genus or species of yeast. In one embodiment, the gene encoding xylose isomerase is from a bacterium. In one embodiment, the bacterium is Clostridium. Streptomyces, Bacteroidetes, Bacteroides, E. coli, or Bacillus. In one embodiment, the recombinant yeast is from the genus Saccharomyces. In one embodiment, the recombinant yeast has at least two copies of the gene encoding xylulokinase. In one embodiment, the recombinant yeast has one copy of the gene encoding xylulokinase. In one embodiment, the recombinant yeast has one copy of the gene encoding xylose isomerase. In one embodiment, the recombinant yeast has at least two copies of the gene encoding xylose isomerase. In one embodiment, the recombinant yeast has one additional copy of the gene encoding transketolase. In one embodiment, the recombinant yeast has at least two additional copies of the gene encoding transketolase. In one embodiment, the recombinant yeast has one additional copy of the gene encoding transaldolase. In one embodiment, the recombinant yeast has at least two additional copies of the gene encoding transaldolase. In one embodiment, the disabling mutation in the gene encoding Isu1 polypeptide comprises a substitution of a tyrosine for the histidine at amino acid residue position 138 of SEQ ID NO:3. In one embodiment, the disabling mutation in the gene encoding Hog1 polypeptide comprises a deletion of the adenine at nucleotide position 844 of SEQ ID NO:7. In one embodiment, the transketolase has at least 80% amino acid sequence identity to SEQ ID NO: 17 or SEQ ID NO:18. In one embodiment, the transaldolase has at least 80% v amino acid sequence identity to SEQ ID NO: 19 or SEQ ID NO:20. In one embodiment, the xylose isomerase has at least 80% v amino acid sequence identity to any one of SEQ ID Nos. 10-12. In one embodiment, the xylulokinase has at least 80% amino acid sequence identity to any one of SEQ ID Nos. 13-16.

Further provided is a yeast inoculum, comprising the recombinant yeast described herein and a culture medium.

Also provided is a method of fermenting a hydrolysate having xylose into ethanol, comprising: contacting under ethanol-producing conditions the recombinant yeast and the hydrolysate for a period of time sufficient to allow fermentation of at least a portion of the hydrolysate into ethanol. In one embodiment, the method further comprises separating the ethanol from fermented hydrolysate. In one embodiment, the method further comprises hydrolyzing a cellulosic material to produce the hydrolysate comprising xylose; and contacting the recombinant yeast to the hydrolysate under conditions that permit fermentation. In one embodiment, the cellulosic material comprises a lignocellulosic biomass. In one embodiment, the lignocellulosic biomass comprises at least one material selected from the group consisting of agricultural residues, wood, municipal solid wastes, paper and pulp industry wastes, and herbaceous crops. In one embodiment, the conditions include aerobic conditions. In one embodiment, the conditions include anaerobic conditions.

In one embodiment, a recombinant yeast is provided that has been genetically engineered to include one or more additional copies of a native transketolase gene (e.g., encoding Tkl1) or a combination of a non-native xylulokinase gene (e.g., XYL3), a non-native xylose isomerase gene (e.g., XYLA), and a native transaldolase gene (e.g., encoding TAL1); and optionally include one or more of a disabling mutation in a gene encoding Cox15; a disabling mutation in a gene encoding Isu1 polypeptide so as to exhibit reduced amounts of functional Isu1 polypeptide, a disabling mutation in a gene encoding Hog1 polypeptide so as to exhibit reduced amounts of functional Hog1 polypeptide, a disabling mutation in a gene encoding Ira2 polypeptide so as to exhibit reduced amounts of functional Ira2 polypeptide, or a disabling mutation in a gene encoding Gre3 polypeptide so as to exhibit reduced amounts of functional Gre3 polypeptide.

In one embodiment, a recombinant yeast is provided that has been genetically engineered to: include at least two additional copies of a native transketolase gene, at least two copies of a non-native xylulokinase gene, at least two copies of a non-native xylose isomerase gene, and at least two additional copies of a native transaldolase gene; and optionally to include one or more of a disabling mutation in a gene encoding Cox15; a disabling mutation in a gene encoding ISU1 polypeptide so as to exhibit reduced amounts of functional Isu1 polypeptide, a disabling mutation in a gene encoding Hog1 polypeptide so as to exhibit reduced amounts of functional Hog1 polypeptide, a disabling mutation in a gene encoding Ira2 polypeptide so as to exhibit reduced amounts of functional Ira2 polypeptide, or a disabling mutation in a gene encoding Gre3 polypeptide so as to exhibit reduced amounts of functional Gre3 polypeptide.

### BRIEF DESCRIPTION OF THE FIGURES

FIG. 1. Adaptive laboratory evolution provided for engineered respiration-deficient yeast strains to ferment xylose into ethanol aerobically. The evolved (Evo1 and Evo2), respiration-deficient (cox15Δ)), and parent containing wild-type COX15 (Parent) strains were cultured aerobically in YPX medium. The evolved strains were evolved from the respiration-deficient strain. Average cell density (A), extracellular xylose (B), and ethanol (C) concentrations from independent triplicate experiments with SEM reported. Parent, cox15Δ, Evo1, and Evo2 indicate strains Y560, Y583, Y1031, and Y1033, respectively (Table 2).

FIG. 2. Evolved strains can ferment xylose into ethanol aerobically in the presence of Antimycin A. The evolved strains (Evo1 and Evo2) containing the empty plasmid (Evo1+Empty or Evo2+Empty) or pRSCENHygMX-COX15 plasmid (Evo1+COX15 or Evo2+COX15 ) were cultured aerobically in YPX+Hygromycin B medium containing a DMSO control or were cultured aerobically 0.5 μg/mL Antimycin A, an inhibitor of Complex III. Average cell density (A and D), extracellular xylose (B and E), and ethanol (C and F) concentrations from independent triplicate experiments with SEM are reported. Evo1, Evo2, Evo1+COX15, and Evo2+COX15 indicate strains Y1031+Empty, Y1033+Empty, Y1031+COX15, and Y1033+COX15, respectively.

FIG. 3. Two independently evolved clones that ferment xylose aerobically contain overlapping duplications in ChrIV and ChrXVI. Average read depths between Y22-3 parental strain (Parent) and the evolved strains (Evo1 and Evo2) were analyzed using sppIDer (Langdon et al., 2018) (A). TAL1-xylA-XYL3 expression cassette and TKL1 gene are duplicated in ChrIV (B) and ChrXVI (C), respectively. Parent, Evo1, and Evo2 indicate strains Y560, Y1031, and Y1033, respectively.

FIG. 4. Duplications of xylA (A-C) and TKL1 (D-F) are responsible for aerobic xylose fermentation by the evolved strains. The evolved strains (Evo1 and Evo2) and the evolved strains with deletion of one copy of xylA or TKL1 were cultured aerobically in YPX medium. Average cell density (A and D), extracellular xylose (B and E), and ethanol (C and F) concentrations from independent triplicate experiments with SEM are reported. Evo1, Evo2, Evo1 xylAxylaΔ, Evo2 xylA/xylaΔ, Evo1 TKL1/tkl1Δ, and Evo2 TKL1/tkl1Δ indicate strains Y1031, Y1033, Y1176, Y1183, Y1185, and Y1189, respectively.

FIG. 5. Duplications of the XYL cassette and TKL1 together with deletion mutations in HOG1, ISU1, and IRA2 enhance fermentation of xylose aerobically and anaerobically. The evolved strains (Evo1 and Evo2) and the engineered strains with deletion of COX15 were cultured aerobically in YPX medium (A-C). The engineered strains with wild-type COX15 were cultured anaerobically in YPX medium (D-F). Average cell density (A and D), extracellular xylose (B and E), and ethanol (C and F) concentrations from independent triplicate experiments with SEM are reported. 3WT indicates a genotype of wild-type HOG1, ISU1, and IRA2.

FIG. 6. Duplications of the XYL cassette and TKL1 increase the xylose conversion rate, while deletion mutations in HOG1, ISU1, and IRA2 reprogram aerobic xylose metabolism from respiration into fermentation. The engineered strains with wild-type COX15 were cultured aerobically in YPX medium. Average cell density (A), extracellular xylose (B), and ethanol (C) concentrations from independent triplicate experiments with SEM are reported. All the strains contain at least one copy of the XYL cassette and TKL1. 3WT indicates a genotype of wild-type HOG1, ISU1, and IRA2.

FIG. 7. Proposed model for aerobic xylose metabolic fluxes, including Crabtree-Warburg-like overflow into fermentation. The wild-type strain contains one copy of the XYL cassette and TKL1 with wild-type copies of HOG1, ISU1, and IRA2. SC, the minimal switching capacity for changing metabolic flux from respiration to fermentation; MC, a minimal capacity for enabling respiratory metabolism; MF, total metabolic flux of the strains. R, respiratory flux; F, fermentative flux.

FIG. 8. Respiration is required for aerobic conversion of xylose into ethanol by the mutant gre3Δhog1Δisu1Δira2Δ xylose-fermenting strain. Respiration-deficient (cox15Δ)), and parent containing wild-type COX15 (Parent) strains were cultured aerobically in YPX medium containing DMSO control or 0.5 μg/mL Antimycin A. Average cell density (A), extracellular xylose (B), and ethanol (C) concentrations from independent triplicate experiments with SEM are reported. Parent and cox15Δ indicate strains Y560 and Y583, respectively (Table 2).

FIG. 9. The engineered respiration-deficient mutant strain adapted to xylose aerobically two independent flasks. The respiration-deficient Parent strain was cultured in three different flasks (A). Two different cultures adapted to xylose aerobically within 19 (B) and 18 (C) generations. The evolved strains (Evo1 and Evo2) were evolved from the respiration-deficient Parent (Y583) strain containing one copy of the XYL cassette with deletion mutations in HOG1, ISU1, GRE3, IRA2, and COX15. Evo1 and Evo2 indicate strains Y1031 and Y1033, respectively.

FIG. 10. Evolved mutations do not enable growth on respiratory carbon sources aerobically. The evolved (Evo1 and Evo2), respiration-deficient (cox15Δ), and Parent containing wild-type COX15 strains were cultured aerobically in YP medium containing glycerol (A) or galactose (B). The evolved strains were evolved from the respiration-deficient strain. Average cell density from independent triplicate experiments with SEM is reported. Parent, cox15Δ, Evo1, and Evo2 indicate strains Y560, Y583, Y1031, and Y1033, respectively.

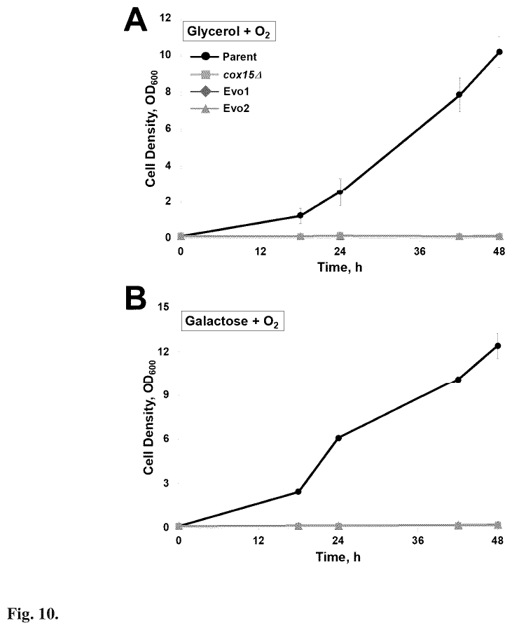

FIG. 11. Evolved mutations do not enable growth on xylose when under glucose-repression. The evolved (Evo1 and Evo2) and parent containing wild-type COX15 (Parent) strains were cultured aerobically in YPX medium with or without 0.5 μg/mL glucosamine. Average cell density (A), extracellular xylose (B), and ethanol (C) concentrations from independent triplicate experiments with SEM are reported. Parent, Evo1, and Evo2 indicate strains Y560, Y1031, and Y1033, respectively.

FIG. 12. Evolved strains ferment xylose into ethanol anaerobically faster than their parent strain. The evolved (Evo1 and Evo2), respiration-deficient (cox15Δ)), and Parent containing wild-type COX15 strains were cultured anaerobically in YPX medium. The evolved strains were evolved from the respiration-deficient strain. Average cell density (A), extracellular xylose (B), and ethanol (C) concentrations from independent triplicate experiments with SEM are reported. Parent, cox15Δ, Evo1, and Evo2 indicate strains Y560, Y583, Y1031, and Y1033, respectively.

FIG. 13. Restoration of COX15 modestly enhances anaerobic xylose fermentation by the evolved strains. The evolved strains (Evo1 and Evo2) transformed with the empty vector control and the evolved strains expressing COX15 (Evo1+COX15 and Evo2+COX15 ) were cultured anaerobically in YPX medium. Average cell density (A), extracellular xylose (B), and ethanol (C) concentrations from independent triplicate experiments with SEM are reported. Evo1, Evo2, Evo1+COX15, and Evo2 +COX15 indicate strains Y1031+Empty, Y1033+Empty, Y1031+COX15, and Y1033+COX15, respectively.

FIG. 14. Evolved strains have different requirements for the extra copy of XYL3 and no requirement for the extra copy of TAL1 in aerobic xylose fermentation. The evolved strains (Evo1 and Evo2) and the evolved strains with a deletion of one copy of XYL3 or TAL1 were cultured aerobically in YPX medium. Average cell density (A, D), extracellular xylose (B, E), and ethanol (C, F) concentrations from independent triplicate experiments with SEM are reported. Asterisk (*) refers to the engineered copy of TAL1 (note that native TAL1 remained in all strains, so strains tested have 2 or 3 copies of TAL1). Evo1, Evo2, Evo1 TAL1*/tal1*Δ, Evo2 TAL1*/tal1*Δ, Evo1 ssXYL3/Ssxyl3Δ, and Evo2 ssXYL3/Ssxyl3Δ indicate strains Y1031, Y1033, Y1174, Y1181, Y1178, and Y1184, respectively.

FIG. 15. Duplication of the XYL cassette alone enables modest xylose fermentation aerobically. The engineered strains with deletion of COX15 were cultured aerobically in YPX medium. Average cell density (A), extracellular xylose (B), and ethanol (C) concentrations from independent triplicate experiments with SEM are reported. All the engineered strains were based on the gre3Δhog1Δisu1Δira2Δ mutant strain containing one copy of the XYL cassette and TKL1 as the Parent strain. cox15Δ, +XYL cox15Δ, +TKL1 cox15Δ, and +XYL +TKL1 cox15Δ, indicate strains Y1370, Y1372, Y1406, and Y1374, respectively.

FIG. 16. Duplications of the XYL cassette and TKL1 enable respiration-dependent growth on xylose aerobically. The engineered strains with wild-type COX15 were cultured aerobically in YPX (A-C) and YPD (D-F) media containing 0.5 μg/mL Antimycin A. Average cell density (A, D), extracellular xylose (B, E), and ethanol (C, F) concentrations from independent triplicate experiments with SEM are reported. All the engineered strains were based on the gre3Δhog1Δisu1Δira2Δ mutant strain containing one copy of the XYL cassette and TKL1 as the Parent strain. 3WT indicates a genotype of wild types HOG1, ISU1, and IRA2. Parent, +XYL +TKL1, and +XYL +TKL1 3WT indicate strains Y560, Y1327, and Y1348, respectively.

FIG. 17. The combination of the duplicated XYL cassette and TKL1 enable faster xylose fermentation anaerobically. The engineered strains with wild-type COX15 were cultured anaerobically in YPX medium. Average cell density (A), extracellular xylose (B), and ethanol (C) concentrations from independent triplicate experiments with SEM are reported. All the engineered strains were based on the gre3Δhog1Δisu1Δira2Δ mutant strain containing one copy of the XYL cassette and TKL1 as the Parent strain. Parent, +XYL, +TKL1, and +XYL +TKL1 indicate strains Y560, Y1228, Y1309, and Y1327, respectively.

FIG. 18. Duplications of the XYL cassette and TKL1 together with deletion mutations in HOG1, ISU1, GRE3, and IRA2 enable faster anaerobic xylose fermentation in the CEN.PK strain background. The engineered CEN.PK113-5D strains with wild-type COX15 were cultured anaerobically in YPX medium. Average cell density (A), extracellular xylose (B), and ethanol (C) concentrations from independent triplicate experiments with SEM are reported. All the engineered CEN.PK113-5D strains were based on the gre3Δhog1Δisu1Δira2Δ mutant strain containing one copy of the XYL cassette and TKL1 as the Parent strain. 3WT indicates a genotype of wild types HOG1, ISU1, and IRA2. Parent, +XYL +TKL1, and +XYL +TKL1 3WT indicate strains Y176, Y1429, and Y1431, respectively.

### DETAILED DESCRIPTION

Bottlenecks in the efficient conversion of xylose into cost-effective biofuels have limited the widespread use of plant lignocellulose as a renewable feedstock. The yeast Saccharomyces cerevisiae ferments glucose into ethanol with such high metabolic flux that it ferments high concentrations of glucose aerobically, a trait called the Crabtree-Warburg Effect. In contrast to glucose, most engineered S. cerevisiae strains do not ferment xylose at economically viable rates and yields, and they require respiration to achieve sufficient xylose metabolic flux and energy return for growth aerobically.

As disclosed herein, respiration-deficient S. cerevisiae strains were evolved to grow on and ferment xylose to ethanol aerobically, a trait analogous to the Crabtree-Warburg Effect for glucose. Through genome sequence comparisons and directed engineering, it was determined that duplications of genes encoding engineered xylose metabolism enzymes, as well as TKL1, a gene encoding a transketolase in the pentose phosphate pathway, were the causative genetic changes for the evolved phenotype. Reengineered duplications of these enzymes, in combination with deletion mutations in HOG1, ISU1, GRE3, and IRA2, increased the rates of aerobic and anaerobic xylose fermentation. Importantly, these genetic modifications function in another genetic background and they increase the rate and yield of xylose-to-ethanol conversion in industrially-relevant switchgrass hydrolysate, indicating that these specific genetic modifications may enable the sustainable production of industrial biofuels from yeast. A model is proposed for how key regulatory mutations prime yeast for aerobic xylose fermentation by lowering the threshold for overflow metabolism, allowing mutations to increase xylose flux and to redirect it into fermentation products.

Thus, the disclosure relates generally to methods and compositions for digesting lignocellulosic material and more particularly to methods that involve exposing the material to yeast variants, e.g., S. cerevisiae variants, having enhanced capacities for anaerobic and aerobic xylose fermentation, e.g., in industrially relevant lignocellulosic hydrolysates.

In a first aspect, provided herein is a recombinant yeast that has been genetically engineered to recombinantly express a xylose isomerase, a xyluokinase, a transaldolase and a transketolase. The recombinantly expressed xylose isomerase may be a native xylose isomerase. The recombinantly expressed xylose isomerase may be a non-native, e.g., from a different species, xylose isomerase. The recombinantly expressed xylulokinase may be a native xylulokinase. The recombinantly expressed xylulokinase may be a non-native xylulokinase. The recombinantly expressed transketolase may be a native transketolase. The recombinantly expressed trasnketolase may be a non-native transketolase. The recombinantly expressed transaldolase may be a native transaldolase. The recombinantly expressed transaldolase may be a non-native transaldolase. In one embodiment, there may be one, two, three or more copies of genes encoding the non-native enzymes. In one embodiment, there may be one, two, three or more additional copies of genes encoding the native enzymes, e.g., for a diploid, the recombinant yeast may have a total of three, four, five or more copies of the gene encoding the native enzyme.

In one embodiment, the recombinant yeast may exhibit reduced amounts of functional Isu1 polypeptide. In one embodiment, the recombinant yeast may exhibit reduced amounts of functional Hog1 polypeptide. In one embodiment, the recombinant yeast may exhibit reduced amounts of functional Ira2 polypeptide. In one embodiment, the recombinant yeast may exhibit reduced amounts of functional Gre3 polypeptide. In one embodiment, the recombinant yeast may exhibit reduced amounts of functional Cox15 polypeptide. The genetically engineered recombinant yeast is capable of increased aerobic xylose fermentation relative to a wild-type yeast or another recombinant yeast not exhibiting reduced amounts of functional Isu1 polypeptide, functional Hog1 polypeptide, functional Ira2 polypeptide, functional Gre3 polypeptide, or functional Cox15 polypeptide, or any combination thereof. Thus, the recombinant yeast may comprise a disabling mutation in a gene encoding Isu1 polypeptide. The disabling mutation may comprise a substitution of a tyrosine for the histidine at amino acid residue position 138 of SEQ ID NO:3. The recombinant yeast may further comprise a disabling mutation in a gene encoding Hog1 polypeptide and exhibit reduced amounts of functional Hog1 polypeptide. The disabling mutation in a gene encoding Isu1 may comprise a substitution of a tyrosine for the histidine at amino acid residue position 138 of SEQ ID NO:3, and the disabling mutation in the gene encoding Hog1 may comprise a deletion of the adenine at nucleotide position 844 of SEQ ID NO:7. The recombinant yeast may be of the genus Saccharomyces. The recombinant yeast may be of the species Saccharomyces cerevisiae. A portion of an extrachromosomal vector stably maintained in the recombinant yeast can comprise the disabling mutation. A nucleic acid sequence comprising the disabling mutation may be integrated into a chromosome of the recombinant yeast.

In another aspect, provided herein is a yeast inoculum comprising a recombinant yeast as provided herein and a culture medium.

In a further aspect, provided herein is a recombinant yeast that has been genetically engineered to recombinantly express a xylose isomerase, a xyluokinase, a transaldolase, and a transketolase. In one embodiment, the recombinant yeast exhibits reduced amounts of functional Isu1 and Hog1 polypeptides, and at least one of functional GRE3, Ira1, and Ira2 polypeptides, and optionally reduced amounts of Cox15. The recombinant yeast may be capable of increased anaerobic xylose fermentation relative to a wild-type yeast or another recombinant yeast not exhibiting reduced amounts of functional Isu1 and Hog1 polypeptides, and at least one of functional GRE3, Ira1, and Ira2 polypeptides. The recombinant yeast can comprise a disabling mutation in a gene encoding Isu1, a disabling mutation in a gene encoding Hog1, and at least one of a disabling mutation in a gene encoding GRE3, a disabling mutation in a gene encoding Ira1, and a disabling mutation in a gene encoding Ira2. The recombinant yeast may exhibit reduced amounts of functional Isu1, Hog1, GRE3, and Ira2 polypeptides and may be capable of increased anaerobic xylose fermentation relative to a wild-type yeast or another recombinant yeast not exhibiting reduced amounts of functional Isu1, Hog1, reE3, and/or Ira2 polypeptides. The disabling mutation in the gene encoding Isu1 can comprise a substitution of a tyrosine for the histidine at amino acid residue position 138 of SEQ ID NO:3; a disabling mutation in the gene encoding Hog1 can comprise a deletion of the adenine at nucleotide position 844 of SEQ ID NO:7; a disabling mutation in the gene encoding Gre3 can comprise a substitution of a threonine for the alanine at amino acid residue position 46 of SEQ ID NO:4; and a disabling mutation in the gene encoding Ira2 can comprise a substitution of a stop codon for the glutamate at amino acid residue at position 2927 of SEQ ID NO:2. The recombinant yeast may be of the genus Saccharomyces. The recombinant yeast may be of the species Saccharomyces cerevisiae. A portion of an extrachromosomal vector stably maintained in the recombinant yeast can comprise the disabling mutations. A nucleic acid sequence comprising the disabling mutations can be integrated into a chromosome of the recombinant yeast.

In another aspect, provided herein is a yeast inoculum comprising a recombinant yeast as provided herein and a culture medium.

In a further aspect, provided herein is a method of fermenting cellulosic material into ethanol. The method can comprise contacting under ethanol-producing conditions a recombinant yeast provided herein to cellulosic material for a period of time sufficient to allow fermentation of at least a portion of the cellulosic material into ethanol. The method can further comprise separating the ethanol from fermented cellulosic material. The method can further comprise hydrolyzing the cellulosic material to produce a hydrolysate comprising xylose; and contacting the recombinant yeast to the hydrolysate under conditions that permit fermentation. The cellulosic material can comprise a lignocellulosic biomass. The lignocellulosic biomass can comprise at least one material selected from the group consisting of agricultural residues, wood, municipal solid wastes, paper and pulp industry wastes, and herbaceous crops.

As can be appreciated, the present disclosure contemplates the use of recombinant yeast as described herein, including certain exemplary recombinant Saccharomyces cerevisiae strains specifically identified herein, for use in the fermentation of xylose-containing cellulosic materials and for production of ethanol or other biofuels and other bioproducts.

### Compositions of the Disclosure

Efficient fermentation of cellulosic feedstock is an essential step in the production of biofuel from plant materials. While S. cerevisiae excels at fermentation of glucose from corn and sugar cane, the fermentation of renewable lignocellulosic biomass presents a significant challenge. Xylose, which is a pentose sugar and a major component of hemicellulose, can comprise almost 30% of total cell wall carbohydrate in grasses. Its conversion, along with glucose, into ethanol is important for any economically-viable cellulosic biofuel process. However, native S. cerevisiae cannot efficiently ferment xylose, as most strains have either lost or downregulated the activities of xylose catabolismproteins. Even when engineered to express the minimal enzymes from native xylose metabolizing organisms, S. cerevisiae is still unable ferment xylose from innocuous lab media at industrially-acceptable rates. However, several Ascomycete yeasts that both ferment and assimilate xylose have been identified, including Pichia stipites (Scheffersomyces stipitis) whose genome has been sequenced. The present disclosure is based, at least in part, on genetic modifications that permit substantially faster xylose fermentation under anaerobic conditions—conditions preferred for industrial ethanol production from plant biomass.

Accordingly, one aspect of the present disclosure relates to strains genetically engineered to be xylose-utilizing and ethanol-producing yeast strains. In particular, the present disclosure provides further genetic modifications to eukaryotic host cells that have been engineered to express xylose metabolism enzymes. Such further genetic modifications improve the efficiency of xylose metabolism in such host cells. In exemplary embodiments, modified host cells of the present disclosure are yeasts that have been additionally genetically engineered for enhanced anaerobic and/or aerobic xylose fermentation and increased ethanol production. The modified host cells are well-suited for producing a variety of fermentation products, including ethanol, in fermentation processes that use xylose or a combination of xylose and glucose as carbon sources.

As used herein, a “host cell” is a cell which has been transformed or transfected, or is capable of transformation or transfection by an exogenous polynucleotide sequence. A host cell that has been transformed or transfected may be more specifically referred to as a “recombinant host cell.” A preferred host cell is a host cell that is naturally capable of alcoholic fermentation, preferably, anaerobic alcoholic fermentation. Host cells may also exhibit a high tolerance to ethanol, low pH, organic acids, and/or elevated temperatures. Such characteristics or activities of the host cell may be naturally present in the host cell or may be introduced or modified by genetic modification.

Exemplary host cells for the present disclosure include yeast cells, particularly yeast cells of the genus Saccharomyces. Preferred yeast species as host cells include Saccharomyces cerevisiae, S. bulderi, S. barnetti, S. exiguus, S. uvarum, S. diastaticus, K. lactis, K. marxianus, and K fragilis, of which yeast cells of the genus Saccharomyces and yeast cells of the species Saccharomyces cerevisiae are preferred. Yeasts of the genus Saccharomyces possess both a metabolic pathway and a fermentative pathway for respiration.

“Yeasts” are eukaryotic micro-organisms classified in the kingdom Fungi. Most reproduce asexually by budding, although some yeasts undergo sexual reproduction by meiosis. Yeasts are unicellular, although some species with yeast forms may become multi-cellular through the formation of a string of connected budding cells known as pseudohyphae, or false hyphae, as seen in most molds. Yeasts do not form a single taxonomic or phylogenetic grouping. The term “yeast” is often taken as a synonym for Saccharomyces cerevisiae, but the phylogenetic diversity of yeasts is illustrated by their assignment to two taxonomic classes of fungi, the ascomycetes and the basidiomycetes. As used herein, wild type yeast refers to a yeast strain designated GLBRCYO (YB-210). GLBRCY0/YB-210 is a strain of S. cerevisiae that can be obtained from the ARS Culture Collection,

National Center for Agricultural Utilization Research, Peoria, Ill., USA; under NRRL YB-210.

A suitable host yeast cell contains at least one native gene (a “xylose isomerase gene”) that produces an active xylose isomerase enzyme that is capable of catalyzing the interconversion of D-xylose to D-xylulose. Xylose isomerase can also catalyze the interconversion of D-ribose to D-ribulose and D-glucose to D-fructose. The enzyme can be specific to the reduction of xylose or non-specific (i.e., capable of catalyzing the conversion of a range of pentose sugars). In some cases, a suitable host yeast cell is genetically engineered to contain an expression cassette containing Clostridium phytofermentans xylose isomerase (CphytoXylA), which can confer anaerobic xylose fermentation by S. cerevisiae with additional genetic modifications (see Brat et al., Applied Environmental Microbiol. 75:2304 (2009)), driven by the ScerTDH3 promoter. In exemplary embodiments, the expression cassette further comprises ScerTAL1 , a Pentose Phosphate Pathway transaldolase enzyme that can improve xylose metabolism when overexpressed (see Ni et al., Applied Environmental Microbiol. 73:2061 (2007); Walfridsson et al., Applied Environmental Microbiol. 61:4184 (1995)), and SstipXy13 driven by the ScerPGK1 and ScerTEF2 promoters, respectively. For example, the host yeast cell can comprise a TAL1-XylA-XYL3 gene expression cassette.

Recombinant yeast of the present disclosure can further comprise genetic modifications intended to delete or disrupt genes encoding certain polypeptides. By “delete or disrupt”, it is meant that the entire coding region of the gene is eliminated (deletion), or the gene or its promoter and/or terminator region is modified (such as by deletion, insertion, or mutation) so that the gene no longer produces an active enzyme, or produces an enzyme with severely reduced activity. The deletion or disruption can be accomplished by genetic engineering methods, forced evolution or mutagenesis, and/or selection or screening.

Recombinant yeast of the present disclosure may comprise genetic modifications that cause reduced levels of, for example, functional Isu1, GRE3, Ira2, Ira1, Cox15, and/or Hog1 polypeptides, or any combination thereof. Isu1 is a polypeptide for mitochondrial iron-sulfur (Fe—S) protein biogenesis. Gre3 is an aldolase enzyme. Hog1 is a mitogen-activated protein (MAP) kinase involved in osmoregulation. Ira1 and Ira2 are Ras GTPase activating proteins that act as a negative regulators of cyclic AMP (cAMP) signaling. Consistent with their role as negative regulators of the Ras-cAMP pathway, disruption of either Ira2 or Ira1 decreases the rate at which Ras proteins hydrolyze GTP to GDP and increases intracellular cAMP levels (Tanaka et al., Mol Cell Biol 9(2):757-68 (1990)). The amino acid sequence of an exemplary Ira2 is set forth as SEQ ID NO:2. The amino acid sequence of an exemplary Gre3 is set forth as SEQ ID NO:4, respectively. The amino acid sequence of an exemplary Isu1 is set forth as SEQ ID NO:3. The amino acid sequence of an exemplary Hog1 is set forth as SEQ ID NO:5.

In some cases, a recombinant yeast of the present disclosure may comprise a disabling mutation that substitutes a threonine amino acid residue for the alanine located amino acid residue position 46 of SEQ ID NO:4, whereby the yeast exhibits a reduced amount of functional Gre3 polypeptide. In other cases, a recombinant yeast of the present disclosure may comprise a disabling mutation that substitutes a stop codon for the glutamate at amino acid residue at position 2927 of SEQ ID NO:2, whereby the yeast exhibits a reduced amount of functional Ira2 polypeptide. Alternatively, a recombinant yeast may comprise a disabling mutation Ira1, whereby the recombinant yeast exhibits a reduced amount of functional Ira1. In other cases, a recombinant yeast of the present disclosure may comprise a disabling mutation that substitutes a tyrosine for the histidine at amino acid residue position 138 of SEQ ID NO:3, whereby the yeast exhibits a reduced amount of functional Isu1 polypeptide. In further cases, a recombinant yeast of the present disclosure may comprise a disabling mutation that deletes an adenine nitrogenous base at nucleotide position 844, e.g., SEQ ID NO:7, of a sequence that encodes SEQ ID NO:5, whereby the deletion causes a codon frame-shift and the yeast exhibits a reduced amount of functional HOG1 polypeptide.

In exemplary embodiments, a recombinant yeast of the disclosure comprises a disabling mutation at each of loci ISU1, GRE3, HOG1, COX15, and IRA2, whereby the mutations result in reduced amounts of functional Isu1, GRE3, Hog1, Cox15 and Ira2 polypeptides, respectively. In some cases, the disabling mutations include a missense mutation in the S. cerevisiae gene encoding Isu1, a missense mutation in the gene encoding GRE3, an aldolase enzyme, a missense mutation in the gene encoding Ira2, and a codon frame-shift mutation in the gene encoding Hog1. Deletion of GRE3 was previously shown to improve xylose fermentation in xylose isomerase-engineered S. cerevisiae strains (Traff et al., Applied and Environmental Microbiol. 67:5668 (2001)). In exemplary embodiments, a recombinant yeast of the present disclosure comprises a disabling mutation at the GRE3 locus that substitutes a threonine for the alanine at amino acid residue position 46 of SEQ ID NO:4; a disabling mutation at the IRA2 locus that substitutes a stop codon for the glutamate at amino acid residue at position 2927 of SEQ ID NO:2; a disabling mutation at the ISU1 locus that substitutes a tyrosine for the histidine at amino acid residue position 138 of SEQ ID NO:3; and a disabling mutation at the HOG1 locus that deletes an adenine nitrogenous base at nucleotide position 844 of a nucleotide sequence, e.g., SEQ ID NO:7, that encodes SEQ ID NO:5. A disabling mutation in the gene encoding COX15 may be accomplished by deleting all or a portion of that gene.

Genetically modified yeasts of the present disclosure containing genetic modifications that reduce or disrupt expression of one or more of Isu1, Hog1, Gre3, Ira2, Cox15, and/or Ira1 polypeptides are useful to ferment xylose pentose sugars to desirable fermentation products such as ethanol. For example, genetically engineered yeast comprising disabling mutations at three loci (e.g., isu1Δhog1Δgre3Δ; isu1Δhog1Δira2Δ; isu1Δhog1Δira1Δ) or four loci (e.g., isu1Δhog1Δgre3Δ, and either ira1Δ or ira2Δ) exhibit substantially faster anaerobic xylose fermentation relative to controls. Anaerobic xylose fermentation was fastest for genetically engineered yeast comprising mutations in four loci (isu1Δhog1 Δgre3Δ, and either ira1Δ or ira2Δ). Recombinant yeast may not comprise null mutations at an IRA1 locus and an IRA2 locus since the double mutation is lethal. For aerobic xylose metabolism, a genetically engineered yeast comprises genetic modifications that reduces or disrupt Isu1 polypeptide expression. Such a genetically engineered yeast may have mutations at additional loci.

It is contemplated that certain additional genetic modifications may produce other desirable characteristics and/or to enable the yeast cell to produce certain products at industrially-acceptable levels.

Genetic modification of the host cell can be accomplished in one or more steps via the design and construction of appropriate vectors and transformation of the host cell with those vectors. Nucleic acid constructs useful in the disclosure may be prepared in conventional ways, by isolating the desired genes from an appropriate host, by synthesizing all or a portion of the genes, or combinations thereof. Similarly, the regulatory signals, the transcriptional and translational initiation and termination regions, may be isolated from a natural source, be synthesized, or combinations thereof. The various fragments may be subjected to endonuclease digestion (restriction), ligation, sequencing, in vitro mutagenesis, primer repair, or the like. The various manipulations are well known in the literature and will be employed to achieve specific purposes.

The various nucleic acids and/or fragments thereof may be combined, cloned, isolated and sequenced in accordance with conventional ways. Standard techniques of molecular biology, microbiology, recombinant DNA, and immunology, which are within the skill of the art, are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

The nucleotides which occur in the various nucleotide sequences appearing herein have their usual single-letter designations (A, G, T, C or U) used routinely in the art. In the present specification and claims, references to Greek letters may either be written out as alpha, beta, etc. or the corresponding Greek letter symbols (e.g., α, β, etc.) may sometimes be used.

The term “isolated nucleic acid” used in the specification and claims means a nucleic acid isolated from its natural environment or prepared using synthetic methods such as those known to one of ordinary skill in the art. Complete purification is not required in either case. The nucleic acids of the disclosure can be isolated and purified from normally associated material in conventional ways such that in the purified preparation the nucleic acid is the predominant species in the preparation. At the very least, the degree of purification is such that the extraneous material in the preparation does not interfere with use of the nucleic acid of the disclosure in the manner disclosed herein. The nucleic acid is preferably at least about 85% pure, more preferably at least about 95% pure and most preferably at least about 99% pure.

Further, an isolated nucleic acid has a structure that is not identical to that of any naturally occurring nucleic acid or to that of any fragment of a naturally occurring genomic nucleic acid spanning more than three separate genes. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene. Specifically excluded from this definition are nucleic acids present in mixtures of clones, e.g., as those occurring in a DNA library such as a cDNA or genomic DNA library. An isolated nucleic acid can be modified or unmodified DNA or RNA, whether fully or partially single-stranded or double-stranded or even triple-stranded. A nucleic acid can be chemically or enzymatically modified and can include so-called non-standard bases such as inosine, as described in a preceding definition.

After each manipulation, the DNA fragment or combination of fragments (polynucleotides) may be inserted into the cloning vector, the vector transformed into a cloning host, e.g., E. coli, the cloning host grown up, lysed, the plasmid isolated and the fragment analyzed by restriction analysis, sequencing, combinations thereof, or the like. “Polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

Targeted integration can be accomplished by designing a vector having regions that are homologous to the upstream (5′-) and downstream (3′-) flanks of the target gene. Either of both of these regions may include a portion of the coding region of the target gene. The gene cassette (including associated promoters and terminators if different from those of the target gene) and selection markers (with associated promoters and terminators as may be needed) can reside on a vector between the regions that are homologous to the upstream and downstream flanks of the target gene. Targeted cassette insertion can be verified by any appropriate method such as, for example, PCR. A host cell may be transformed according to conventional methods that are known to practitioners in the art. Electroporation and/or chemical (such as calcium chloride- or lithium acetate-based) transformation methods can be used. The DNA used in the transformations can either be cut with particular restriction enzymes or used as circular DNA. Methods for transforming yeast strains are described in WO 99/14335, WO 00/71738, WO 02/42471, WO 03/102201, WO 03/102152 and WO 03/049525; these methods are generally applicable for transforming host cells in accordance with this disclosure. Other methods for transforming eukaryotic host cells are well known in the art such as from standard handbooks, such as Sambrook and Russel (2001) “Molecular Cloning: A Laboratory Manual (3rd edition),” Cold Spring Harbor Laboratory, Cold Spring Harbor Laboratory Press, or F. Ausubel et al., eds., “Current protocols in molecular biology,” Green Publishing and Wiley Interscience, New York (1987).

In another aspect, compositions of the present disclosure include yeast inoculums comprising recombinant yeast as provided herein. A yeast inoculum of the present disclosure can comprise a recombinant yeast as provided herein and (b) a culture medium. In exemplary embodiments, the recombinant yeast is S. cerevisiae and the culture medium is a liquid culture medium. Yeast inocula of the present disclosure include large-scale preparations of sufficient quantities of viable yeast cells for use in, for example, xylose fermentation and other industrial ethanol-producing methods. A yeast inoculum of the present disclosure can be contacted to cellulosic material for xylose fermentation.

### Methods of the Disclosure

The methods provided by the present disclosure involve the incorporation of genetic modifications into a host organism, e.g., incorporating genes encoding certain polypeptides into a single host organism, and the use of those organisms to convert xylose to ethanol. In particular, the present disclsoure provides methods of fermenting cellulosic material comprising the 5-carbon sugar xylose under anaerobic or aerobic conditions, where the method comprises use of a recombinant yeast.

In exemplary embodiments, recombinant yeast of the present disclosure are used to make a useful fuel (e.g., ethanol) or plant material-derived chemical feedstock by converting xylose and other sugars under appropriate fermentation conditions. The sugars can come from a variety of sources including, but not limited to, cellulosic material. The cellulosic material can be lignocellulosic biomass. As used herein, the term “lignocellulosic biomass” refers to any materials comprising cellulose, hemicellulose, and lignin, wherein the carbohydrate polymers (cellulose and hemicelluloses) are tightly bound to the lignin. Generally, lignocellulosic material for making ethanol is feedstock such as corn stover, which consists of the stems, cobs, and leaves from the corn plants (i.e., the non-grain material). Corn stover is typically shredded by mechanical means and incorporated by tillage into topsoil for decomposition. In addition to lignocellulosic ethanol production from corn stover, other feedstocks such as sorghum, wheat, or another grain can be used. In some cases, lignocellulosic biomass comprises material selected from the group consisting of materials that comprise at least 75% cellulose, cellulose/hemicelluloses, xylose, biomass, and chitin. In other cases, the lignocellulosic biomass comprises at least one material selected from the group consisting of agricultural residues, wood, municipal solid wastes, paper and pulp industry wastes, and herbaceous crops. As used herein, the term “biomass” refers to a renewable energy source, is biological material from living or recently living organisms. As an energy source, biomass can either be used directly, or converted into other energy products such as biofuel. Biomass includes plant or animal matter that can be converted into fibers or other industrial chemicals, including biofuels. Industrial biomass can be grown from numerous types of plants, including miscanthus, switchgrass, hemp, corn, poplar, willow, sorghum, sugarcane, bamboo, and a variety of tree species, ranging from eucalyptus to oil palm (palm oil). Thus, biomass can include wood biomass and non-wood biomass.

In some cases, cellulosic material is contacted with one or more of the genetically engineered yeasts disclosed herein (e.g., a yeast strain genetically modified to exhibit reduced amounts of functional Isu1 , GRE3, Hog1, Ira1, Cox15, and/or Ira2 polypeptides) under anaerobic or aerobic conditions. For example, a method of fermenting cellulosic material can comprise contacting under anaerobic conditions a recombinant yeast as provided herein to cellulosic material for a period of time sufficient to allow fermentation of at least a portion of the cellulosic material. In exemplary embodiments, a recombinant yeast used according to the methods provided herein is Saccharomyces cerevisiae.

The fermentation process may be an aerobic or an anaerobic fermentation process. Anaerobic fermentation is herein defined as a fermentation process run in the absence of oxygen or in which substantially no oxygen is consumed, preferably less than 5, 2.5 or 1 mmol/L/h, e.g., 0 mmol/L/h is consumed (i.e., oxygen consumption is not detectable), and where organic molecules serve as both electron donor and electron acceptors. In the absence of oxygen, NADH produced in glycolysis and biomass formation cannot be oxidized by oxidative phosphorylation.

In some cases, the method can include a first hydrolyzation step. For example, when cellulosic material is used in the methods disclosed herein, the material can be hydrolyzed to produce a hydrolysate comprising xylose, which is subsequently contacted to one or more recombinant yeasts of the present disclosure. As used herein, the term “hydrolysate” refers to a fermentable sugar-containing product produced from cellulosic material (e.g., biomass), typically through pretreatment and saccharification processes. In general, cellulosic material is pretreated using thermal, physical, and/or chemical treatments, and saccharified enzymatically. Physical and chemical treatments may include grinding, milling, cutting, base treatment such as with ammonia or NaOH, and acid treatment. In some cases, plant biomass can be pretreated using AFEX™. While highly effective at pretreating grasses for enzymatic hydrolysis, AFEX™ pretreament generates diverse inhibitory compounds from corn stover that impair xylose fermentation (Schwalbach et al., Applied Environ. Microbiol. 78:3442 (2012); Koppram et al., Biotechnol. Biofuels 5:32 (2012); Lau & Dale, PNAS USA 106:1368 (2009)). The inhibitory compounds are degradation products derived from plant cell walls such as hemicellulose and lignin-derived acetate and aromatic molecules.

Enzymatic saccharification typically makes use of an enzyme composition or blend to break down cellulose and/or hemicellulose and to produce a hydrolysate containing 6-carbon sugars (e.g., glucose) and 5-carbon sugars (e.g., xylose, arabinose). For review of saccharification enzymes, see Lynd et al., Microbiol. Mol. Biol. Rev. 66:506-577 (2002). Saccharification enzymes may be obtained commercially. In some cases, saccharification enzymes may be produced using recombinant microorganisms that have been engineered to express one or more saccharifying enzymes.

In some cases, methods of the present disclosure further comprise an ethanol separation or extraction step. Following conversion of sugars into ethanol, the ethanol can be separated from a fermentation culture using, for example, a standard distillation method or by filtration using membranes or membrane systems known in the art. Methods of separating or extracting are not restricted to those disclosed herein.

Methods of the present disclosure can be conducted continuously, batch-wise, or some combination thereof.

In another aspect, provided herein are methods for producing fuels and chemical feedstocks from glycerol (or glycerin). Glycerol is a by-product of biodiesel production, which, using a recombinant yeast of the present disclosure, could be further converted to a fuel or chemical feedstock such as, for example, ethanol, lactic acid, isobutanol, and propanediol. In some cases, a method of converting glycerol to ethanol can comprise contacting glycerol to one or more of the genetically engineered yeasts disclosed herein (e.g., a yeast strain genetically modified to exhibit reduced amounts of functional Isu1, GRE3, Hog1, Ira1, Cox15, and/or Ira2 polypeptides) under appropriate fermentation conditions. In exemplary embodiments, methods are provided for producing lactic acid from glycerol. In such cases, the method comprises contacting under anaerobic conditions a recombinant yeast provided herein to glycerol for a period of time sufficient to allow fermentation of at least a portion of the glycerol into lactic acid. Lactic acid is in high demand as a chemical feedstock for the biodegradable plastic known as polylactic acid (PLA), a biopolymer that is useful in a variety of applications including packaging material and medical devices (e.g., surgical sutures, orthopedic implants). The raw materials to manufacture lactic acid are expensive and limit use of PLA. In other cases, the method of converting glycerol into a useful fuel comprises contacting under anaerobic conditions a recombinant yeast as provided herein to glycerol for a period of time sufficient to allow fermentation of at least a portion of the glycerol into ethanol or butanol.

In exemplary embodiments, a recombinant yeast used according to the methods provided herein is Saccharomyces cerevisiae (S. cerevisiae). Following conversion of glycerol into ethanol, the fuel or chemical feedstock can be separated from a fermentation culture using, for example, a standard distillation method or by filtration using membranes or membrane systems known in the art. Methods of separating or extracting are not restricted to those disclosed or exemplified herein.

### Articles of Manufacture

In a further aspect, the present disclosure provides an article of manufacture containing any one or more of the recombinant yeasts disclosed herein is provided. An article of manufacture can contain one of the microorganisms disclosed herein (e.g., one or more of the yeast strains), or an article of manufacture can contain two or more of the microorganisms disclosed herein. Articles of manufacture disclosed herein also can include, for example, components necessary for growth of the particular microorganism(s).

It is understood that this disclosure is not limited to the particular methodology, protocols, materials, and reagents described, as these may vary. It is also to be understood that the terminology used herein is for the purpose of describing particular embodiments only, and is not intended to limit the scope of the present disclosure, which will be limited only by the appended claims.

It must be noted that as used herein and in the appended claims, the singular forms “a,” “an,” and “the” include plural reference unless the context clearly dictates otherwise. As well, the terms “a” (or “an”), “one or more” and “at least one” can be used interchangeably herein. It is also to be noted that the terms “comprising,” “including,” and “having” can be used interchangeably.

### Exemplary Sequences

Exemplary xylose isomerase sequences for use in the host cells and methods of the disclosure include but are not limited to

(SEQ ID NO: 10)
mknyfpnvpe vkyegpnstn pfafkyydae rivagktmke
hcrfalswwh tlcaggadpf gvttmdrsyg nitdpmefak
akvdagfelm tklgieyfcf hdadiapege nfeeskknlf
vivdyikekm dqtgikllwg tannfghprf mhgastscna
dvfayaaaki knaldatikl ggkgyvfwgg regyetllnt
dlgleldnma rlmkmaveyg rangfdgdfy iepkpkeptk
hqydfdtatv lgflrkygle kdfkmniean hatlaghtfe
helalarvng vfgsvdanqg dpnlgwdtdq fptdvhsatl
amlevlkagg ftngglnfda kvrrgsfefd diaygyiagm
dtfalglika aeiiedgria kfvedryasy ktgigkaivd
gttsleeleq yvlthnepvm qsgrqevles ivnnilfr,
(SEQ ID NO: 11)
mknyfpnvpe vkyegpnstn pfafkyydan kvvagktmke
hcrfalswwh tlcaggadpf gvttmdrtyg nitdpmelak
akvdagfelm tklgieffcf hdadiapegd tfeeskknlf
eivdyikekm dqtgikllwg tannfshprf mhgastscna
dvfayaaaki knaldatikl ggkgyvfwgg regyetllnt
dlgleldnma rlmkmaveyg rangfdgdfy iepkpkeptk
hqydfdtatv laflrkygle kdfkmniean hatlaghtfe
helamarvng afgsvdanqg dpnlgwdtdq fptdvhsatl
amlevlkagg ftngglnfda kvrrgsfefd diaygyiagm
dtfalglika aeiiddgria kfvddryasy ktgigkaivd
gttsleeleq yvlthsepvm qsgrqevlet ivnnilfr,
(SEQ ID NO: 12)
MKNYFPNVPEVKYEGPNSTNPFAFKYYDANKVVAGKTMKEHCRFALSWWH
TLCAGGADPFGVTTMDRTYGNITDPMELAKAKVDAGFELMTKLGIEFFCF
HDADIAPEGDTFEESKKNLFEIVDYIKEKMDQTGIKLLWGTANNFSHPRF
MHGASTSCNADVFAYAAAKIKNALDATIKLGGKGYVFWGGREGYETLLNT
DLGLELDNMARLMKMAVEYGRANGFDGDFYIEPKPKEPTKHQYDFDTATV
LAFLRKYGLEKDFKMNIEANHATLAGHTFEHELAMARVNGAFGSVDANQG
DPNLGWDTDQFPTDVHSATLAMLEVLKAGGFTNGGLNFDAKVRRGSFEFD
DIAYGYIAGMDTFALGLIKAAEIIDDGRIAKFVDDRYASYKTGIGKAIVD
GTTSLEELEQYVLTHSEPVMQSGRQEVLETIVNNILFR,

or a polypeptide having at least 80%, 82%, 85%, 86%, 88%, 90%, 92%, 94%, 95%, 97%, 98% or 99% amino acid sequence identity thereto.

Exemplary xylulokinase sequences for use in the host cells and methods of the disclosure include but are not limited to

(SEQ ID NO: 13)
mtttpfdapd klflgfdlst qqlkiivtde nlaalktynv
efdsinssvq kgviaindei skgaiispvy mwldaldhvf
edmkkdgfpf nkvvgisgsc qqhgsvywsr taekvlseld
aesslssqmr saftfkhapn wqdhstgkel eefervigad
aladisgsra hyrftglqir klstrfkpek ynrtarislv
ssfvasvllg ritsieeada cgmnlydiek refneellai
aagvhpeldg veqdgeiyra ginelkrklg pvkpityese
gdiasyfvtr ygfnpdckiy sftgdnlati islplapnda
lislgtsttv liitknyaps sqyhlfkhpt mpdhymgmic
ycngslarek vrdevnekfn vedkkswdkf neildkstdf
nnklgiyfpl geivpnaaaq ikrsvlnskn eivdvelgdk
nwqpeddvss ivesqtlscr lrtgpmlsks gdssasssas
pqpegdgtdl hkvyqdlvkk fgdlytdgkk qtfesltarp
nrcyyvggas nngsiirkmg silapvngny kvdipnacal
ggaykaswsy eceakkewig ydqyinrlfe vsdemnsfev
kdkwleyang vgmlakmese lkh,
(SEQ ID NO: 14)
mtttpfdapd klflgfdlst qqlkiivtde nlaalktynv
efdsinssvq kgviaindei skgaiispvy mwldaldhvf
edmkkdgfpf nkvvgisgsc qqhgsvywsr taekvlseld
aesslssqmr saftfkhapn wqdhstgkel eefervigad
aladisgsra hyrftglqir klstrfkpek ynrtarislv
ssfvasvllg ritsieeada cgmnlydiek refneellai
aagvhpeldg veqdgeiyra ginelkrklg pvkpityese
gdiasyfvtr ygfnpdckiy sftgdnlati islplapnda
lislgtsttv liitknyaps sqyhlfkhpt mpdhymgmic
ycngslarek vrdevnekfn vedkkswdkf neildkstdf
nnklgiyfpl geivpnaaaq ikrsvlnskn eivdvelgdk
nwqpeddvss ivesqtlscr lrtgpmlsks gdssasssas
pqpegdgtdl hkvyqdlvkk fgdlytdgkk qtfesltarp
nrcyyvggas nngsiirkmg silapvngny kvdipnacal
ggaykaswsy eceakkewig ydqyinrlfe vsdemnsfev
kdkwleyang vgmlakmese lkh,
(SEQ ID NO: 15)
mtttpfdapd klflgfdlst qqlkiivtde nlaalktynv
efdsinssvq kgviaindei skgaiispvy mwldaldhvf
edmkkdgfpf nkvvgisgsc qqhgsvywsr taekvlseld
aesslssqmr saftfkhapn wqdhstgkel eefervigad
aladisgsra hyrftglqir klstrfkpek ynrtarislv
ssfvasvllg ritsieeada cgmnlydiek refneellai
aagvhpeldg veqdgeiyra ginelkrklg pvkpityese
gdiasyfvtr ygfnpdckiy sftgdnlati islplapnda
lislgtsttv liitknyaps sqyhlfkhpt mpdhymgmic
ycngslarek vrdevnekfn vedkkswdkf neildkstdf
nnklgiyfpl geivpnaaaq ikrsvlnskn eivdvelgdk
nwqpeddvss ivesqtlscr irtgpmlsks gdssasssas
pqpegdgtdl hkvyqdlvkk fgdlftdgkk qtfesltarp
nrcyyvggas nngsiixkmg silapvngny kvdipnacal
ggaykaswsy eceakkewig ydqyinrlfe vsdemnsfev
kdkwleyang vgmlakmese lkh,
(SEQ ID NO: 16)
MTTTPFDAPDKLFLGFDLSTQQLKIIVTDENLAALKTYNVEFDSINSSVQ
KGVIAINDEISKGAIISPVYMWLDALDHVFEDMKKDGFPFNKVVGISGSC
QQHGSVYWSRTAEKVLSELDAESSLSSQMRSAFTFKHAPNWQDHSTGKEL
EEFERVIGADALADISGSRAHYRFTGLQIRKLSTRFKPEKYNRTARISLV
SSFVASVLLGRITSIEEADACGMNLYDIEKREFNEELLAIAAGVHPELDG
VEQDGEIYRAGINELKRKLGPVKPITYESEGDIASYFVTRYGFNPDCKIY
SFTGDNLATIISLPLAPNDALISLGTSTTVLIITKNYAPSSQYHLFKHPT
MPDHYMGMICYCNGSLAREKVRDEVNEKFNVEDKKSWDKFNEILDKSTDF
NNKLGIYFPLGEIVPNAAAQIKRSVLNSKNEIVDVELGDKNWQPEDDVSS
IVESQTLSCRLRTGPMLSKSGDSSASSSASPQPEGDGTDLHKVYQDLVKK
FGDLYTDGKKQTFESLTARPNRCYYVGGASNNGSIIRKMGSILAPVNGNY
KVDIPNACALGGAYKASWSYECEAKKEWIGYDQYINRLFEVSDEMNSFEV
KDKWLEYANGVGMLAKMESELKH,

or a polypeptide having at least 80%, 82%, 85%, 86%, 88%, 90%, 92%, 94%, 95%, 97%, 98% or 99% amino acid sequence identity thereto.

Exemplary transketolase (TKL) sequences for use in the host cells and methods of the disclosure include but are not limited to

(SEQ ID NO: 17)
mtqftdidkl avstirilav dtvskansgh pgaplgmapa
ahvlwsqmrm nptnpdwinr drfvlsngha vallysmlhl
tgydlsiedl kqfrqlgsrt pghpefelpg vevttgplgq
gisnavgmam aqanlaatyn kpgftlsdny tyvflgdgcl
qegisseass laghlklgnl iaiyddnkit idgatsisfd
edvakryeay gwevlyveng nedlagiaka iaqaklskdk
ptlikmttti gygslhagsh svhgaplkad dvkqlkskfg
fnpdksfvvp qevydhyqkt ilkpgveann kwnklfseyq
kkfpelgael arrlsgqlpa nwesklptyt akdsavatrk
lsetvledvy nqlpeliggs adltpsnltr wkealdfqpp
ssgsgnysgr yirygireha mgaimngisa fganykpygg
tflnfvsyaa gavrlsalsg hpviwvathd sigvgedgpt
hqpietlahf rslpniqvwr padgnevsaa yknsleskht
psiialsrqn lpqlegssie saskggyvlq dvanpdiilv
atgsevslsv eaaktlaakn ikarvvslpd fftfdkqple
yrlsvlpdnv pimsvevlat tcwgkyahqs fgidrfgasg
kapevfkffg ftpegvaera qktiafykgd klisplkkaf,
(SEQ ID NO: 18)
MTQFTDIDKLAVSTIRILAVDTVSKANSGHPGAPLGMAPAAHVLWSQMRM
NPTNPDWINRDRFVLSNGHAVALLYSMLHLTGYDLSIEDLKQFRQLGSRT
PGHPEFELPGVEVTTGPLGQGISNAVGMAMAQANLAATYNKPGFTLSDNY
TYVFLGDGCLQEGISSEASSLAGHLKLGNLIAIYDDNKITIDGATSISFD
EDVAKRYEAYGWEVLYVENGNEDLAGIAKAIAQAKLSKDKPTLIKMTTTI
GYGSLHAGSHSVHGAPLKADDVKQLKSKFGFNPDKSFVVPQEVYDHYQKT
ILKPGVEANNKWNKLFSEYQKKFPELGAELARRLSGQLPANWESKLPTYT
AKDSAVATRKLSETVLEDVYNQLPELIGGSADLTPSNLTRWKEALDFQPP
SSGSGNYSGRYIRYGIREHAMGAIMNGISAFGANYKPYGGTFLNFVSYAA
GAVRLSALSGHPVIWVATHDSIGVGEDGPTHQPIETLAHFRSLPNIQVWR
PADGNEVSAAYKNSLESKHTPSIIALSRQNLPQLEGSSIESASKGGYVLQ
DVANPDIILVATGSEVSLSVEAAKTLAAKNIKARVVSLPDFFTFDKQPLE
YRLSVLPDNVPIMSVEVLATTCWGKYAHQSFGIDRFGASGKAPEVFKFFG
FTPEGVAERAQKTIAFYKGDKLISPLKKAF,

or a polypeptide having at least 80%, 82%, 85%, 86%, 88%, 90%, 92%, 94%, 95%, 97%, 98% or 99% amino acid sequence identity thereto.

Exemplary transaldolase (Tal) sequences for use in the host cells and methods of the disclosure include but are not limited to

(SEQ ID NO: 19)
msepaqkkqk vannsleqlk asgtvvvadt gdfgsiakfq
pqdsttnpsl ilaaakqpty aklidvavey gkkhgkttee
qvenavdrll vefgkeilki vpgrvstevd arlsfdtqat
iekarhiikl feqegvsker vlikiastwe giqaakelee
kdgihcnltl lfsfvqavac aeaqvtlisp fvgrildwyk
sstgkdykge adpgvisvkk iynyykkygy ktivmgasfr
stdeiknlag vdyltispal ldklmnstep fprvldpvsa
kkeagdkisy isdeskfrfd lnedamatek lsegirkfsa
divtlfdlie kkvta,
(SEQ ID NO: 20)
MSEPAQKKQKVANNSLEQLKASGTVVVADTGDFGSIAKFQPQDSTTNPSL
ILAAAKQPTYAKLIDVAVEYGKKHGKTTEEQVENAVDRLLVEFGKEILKI
VPGRVSTEVDARLSFDTQATIEKARHIIKLFEQEGVSKERVLIKIASTWE
GIQAAKELEEKDGIHCNLTLLFSFVQAVACAEAQVTLISPFVGRILDWYK
SSTGKDYKGEADPGVISVKKIYNYYKKYGYKTIVMGASFRSTDEIKNLAG
VDYLTISPALLDKLMNSTEPFPRVLDPVSAKKEAGDKISYISDESKFRFD
LNEDAMATEKLSEGIRKFSADIVTLFDLIEKKVTA,

or a polypeptide having at least 80%, 82%, 85%, 86%, 88%, 90%, 92%, 94%, 95%, 97%, 98% or 99% amino acid sequence identity thereto.

Exemplary Ira2 sequences for use, e.g., to decrease or disable expression, in the host cells and methods of the disclosure include but are not limited to

(SEQ ID NO: 2)
msqptknkkk ehgtdskssr mtrtlvnhil ferilpilpv
esnlstysev eeyssfiscr svlinvtvsr danamvegtl
eliesllqgh eiisdkgssd viesiliilr llsdaleynw
qnqeslhynd isthvehdqe qkyrpklnsi lpdyssthsn
gnkhffhqsk pqalipelas kllescaklk fntrtlqilq
nmishvhgni lttlsssilp rhksyltrhn hpshckmids
tlghilrfva asnpseyfef irksvqvpvt qththshshs
hslpssvyns ivphfdlfsf iylskhnfkk yleliknlsv
tlrktiyhcl llhysakaim fwimarpaey yelfnllkdn
nnehskslnt lnhtlfeeih stfnvnsmit tnqnahqgss
spsssspssp psssssdnnn qniiakslsr qlshhqsyiq
qqserklhss wttnsqssts lssstsnstt tdfsthtqpg
eydpslpdtp tmsnitisas sllsqtptpt tqlqqrlnsa
aaaaaaaasp snstptgyta eqqsrasyda hktghtgkdy
dehflsvtrl dnvlelythf ddtevlphts vlkflttltm
fdidlfneln atsfkyipdc tmhrpkerts sfnntahetg
sektsgikhi tqglkkltsl psstkktvkf vkmllrnlng
nqavsdvall dtmrallsff tmtsavflvd rnlpsvlfak
rlipimgtnl svgqdwnski nnslmvclkk nsttfvqlql
iffssaiqfd helllarlsi dtmannlnmq klclytegfr
iffdipskke lrkaiavkis kffktlfsii adillqefpy
fdeqitdiva sildgtiine ygtkkhfkgs spslcsttrs
rsgstsqssm tpvsplgldt dicpmntlsl vgsstsrnsd
nvnslnsspk nlssdpylsh lvaprarhal ggpssiirnk
ipttltsppg teksspvqrp qtesisatpm aitnstplss
aafgirsplq kirtrrysde slgkfmkstn nyiqehlipk
dlneatlqda rriminifsi fkrpnsyfii phninsnlqw
vsqdfrnimk pifvaivspd vdlqntaqsf mdtllsnvit
ygesdenisi egyhllcsyt vtlfamglfd lkinnekrqi
llditvkfmk vrshlagiae ashhmeyisd sekltfplim
gtvgralfvs lyssqqkiek tlkiayteyl sainfherni
ddadktwvhn iefveamchd nyttsgsiaf qrrtrnnilr
fatipnaill dsmrmiykkw htythsksle kqerndfrnf
agilaslsgi lfinkkilqe mypylldtvs elkknidsfi
skqcqwlnyp dlltrensrd ilsvelhpls fnllfnnlrl
klkelacsdl sipenessyv lleqiikmlr tilgrdddny
vmmlfsteiv dlidlltdei kkipaycpky lkaiiqmtkm
fsalqhsevn lgvknhfhvk nkwirqitdw fqvsiareyd
fenlskplke mdlvkrdmdi lyidtaieas taiayltrht
fleippaasd pelsrsrsvi fgfyfnilmk glekssdrdn
ypvflrhkms vlndnvilsl tnlsntnvda slqftlpmgy
sgnrnirnaf levfinivtn yrtytaktdl gkleaadkfl
rytiehpqls sfgaavcpas didayaagli nafetrnath
ivvaqlikne iekssrptdi lrrnscatrs lsmlarskgn
eylirtlqpl lkkiiqnrdf feieklkped sdaerqielf
vkymnelles isnsvsyfpp plfyicqniy kvacekfpdh
aiiaagsfvf lrffcpalvs pdseniidis hlsekrtfis
lakviqnian gsenfsrwpa lcsqkdflke csdrifrfla
elcrtdrtid iqvrtdptpi afdyqflhsf vylyglevrr
nvlneakhdd gdidgddfyk ttflliddvl gqlgqpkmef
sneipiyire hmddypelye fmnrhafrni etstayspsv
hestssegip iitltmsnfs drhvdidtva ykflqiyari
wttkhcliid ctefdeggld mrkfislvmg llpevapknc
igcyyfnvne tfmdnygkcl dkdnvyvssk iphyfinsns
deglmksvgi tgqglkvlqd irvslhditl ydekrnrftp
vslkigdiyf qvlhetprqy kirdmgtlfd vkfndvyeis
rifevhvssi tgvaaeftvt fqderrlifs spkyleivkm
fyyaqirles eyemdnnsst sspnsnnkdk qqkertkllc
hlllvsligl fdeskkmkns synliaatea sfglnfgshf
hrspevyvpe dtttflgvig kslaesnpel taymfiyvle
alknnviphv yiphticgls ywipnlyqhv yladdeegpe
nishifrili rlsvretdfk avymqyvwll llddgrltdi
ivdevinhal erdsenrdwk ktislltvlp ttevanniiq
kilakirsfl pslkleamtq swseltilvk isihvffets
llvqmylpei lfivsllidv gprelrsslh qllmnvchsl
ainsalpqdh rnnldeisdi fahqkvkfmf gfsedkgril
qifsassfas kfnildffin nilllmeyss tyeanvwktr
ykkyvlesvf tsnsflsars imivgimgks yiteglckam
lietmkviae pkitdehlfl aishiftysk ivegldpnld
lmkhlfwfst lflesrhpii fegallfvsn cirrlymaqf
enesetslis tllkgrkfah tflskienls givwnednft
hilifiinkg lsnpfiksta fdflkmmfrn syfehqinqk
sdhylcymfl lyfvlncnqf eellgdvdfe gemvnienkn
tipkillewl ssdnenanit lyqgailfkc svtdepsrfr
faliirhllt kkpicalrfy svirneirki safeqnsdcv
plafdilnll vthsesnsle klheesierl tkrglsivts
sgifaknsdm mipldvkped iyerkrimtm ilsrmscsa,

or a polypeptide having at least 80%, 82%, 85%, 86%, 88%, 90%, 92%, 94%, 95%, 97%, 98% or 99% amino acid sequence identity thereto.

Exemplary sequences to decrease or disable expression of Isu l in the host cells of the disclosure include but are not limited to sequences that encode

(SEQ ID NO: 3)
Met Leu Pro Val Ile Thr Arg Phe Ala Arg Pro Ala
Leu Met Ala Ile Arg Pro Val Asn Ala Met Gly Val
Leu Arg Ala Ser Ser Ile Thr Lys Arg Leu Tyr His
Pro Lys Val Ile Glu His Tyr Thr His Pro Arg Asn
Val Gly Ser Leu Asp Lys Lys Leu Pro Asn Val Gly
Thr Gly Leu Val Gly Ala Pro Ala Cys Gly Asp Val
Met Arg Leu Gln Ile Lys Val Asn Asp Ser Thr Gly
Val Ile Glu Asp Val Lys Phe Lys Thr Phe Gly Cys
Gly Ser Ala Ile Ala Ser Ser Ser Tyr Met Thr Glu
Leu Val Gln Gly Met Thr Leu Asp Asp Ala Ala Lys
Ile Lys Asn Thr Glu Ile Ala Lys Glu Leu Ser Leu
Pro Pro Val Lys Leu His Cys Ser Met Leu Ala Glu
Asp Ala Ile Lys Ala Ala Ile Lys Asp Tyr Lys Ser
Lys Arg Asn Thr Pro Thr Met Leu Ser,

or a polypeptide having at least 80%, 82%, 85%, 86%, 88%, 90%, 92%, 94%, 95%, 97%, 98% or 99% amino acid sequence identity thereto.

Exemplary sequences to decrease or disable expression of Gre3 in the host cells of the disclosure include but are not limited to sequence that encode

(SEQ ID NO: 4)
Met Ser Ser Leu Val Thr Leu Asn Asn Gly Leu Lys
Met Pro Leu Val Gly Leu Gly Cys Trp Lys Ile Asp
Lys Lys Val Cys Ala Asn Gln Ile Tyr Glu Ala Ile
Lys Leu Gly Tyr Arg Leu Phe Asp Gly Ala Cys Asp
Tyr Gly Asn Glu Lys Glu Val Gly Glu Gly Ile Arg
Lys Ala Ile Ser Glu Gly Leu Val Ser Arg Lys Asp
Ile Phe Val Val Ser Lys Leu Trp Asn Asn Phe His
His Pro Asp His Val Lys Leu Ala Leu Lys Lys Thr
Leu Ser Asp Met Gly Leu Asp Tyr Leu Asp Leu Tyr
Tyr Ile His Phe Pro Ile Ala Phe Lys Tyr Val Pro
Phe Glu Glu Lys Tyr Pro Pro Gly Phe Tyr Thr Gly
Ala Asp Asp Glu Lys Lys Gly His Ile Thr Glu Ala
His Val Pro Ile Ile Asp Thr Tyr Arg Ala Leu Glu
Glu Cys Val Asp Glu Gly Leu Ile Lys Ser Ile Gly
Val Ser Asn Phe Gln Gly Ser Leu Ile Gln Asp Leu
Leu Arg Gly Cys Arg Ile Lys Pro Val Ala Leu Gln
Ile Glu His His Pro Tyr Leu Thr Gln Glu His Leu
Val Glu Phe Cys Lys Leu His Asp Ile Gln Val Val
Ala Tyr Ser Ser Phe Gly Pro Gln Ser Phe Ile Glu
Met Asp Leu Gln Leu Ala Lys Thr Thr Pro Thr Leu
Phe Glu Asn Asp Val Ile Lys Lys Val Ser Gln Asn
His Pro Gly Ser Thr Thr Ser Gln Val Leu Leu Arg
Trp Ala Thr Gln Arg Gly Ile Ala Val Ile Pro Lys
Ser Ser Lys Lys Glu Arg Leu Leu Gly Asn Leu Glu
Ile Glu Lys Lys Phe Thr Leu Thr Glu Gln Glu Leu
Lys Asp Ile Ser Ala Leu Asn Ala Asn Ile Arg Phe
Asn Asp Pro Trp Thr Trp Leu Asp Gly Lys Phe Pro
Thr Phe Ala,

or a polypeptide having at least 80%, 82%, 85%, 86%, 88%, 90%, 92%, 94%, 95%, 97%, 98% or 99% amino acid sequence identity thereto.

Exemplary sequences to decrease or disable expression of Hog1 in the host cells of the disclosure include but are not limited to sequences that encode

(SEQ ID NO: 5)
Met Thr Thr Asn Glu Glu Phe Ile Arg Thr Gln Ile
Phe Gly Thr Val Phe Glu Ile Thr Asn Arg Tyr Asn
Asp Leu Asn Pro Val Gly Met Gly Ala Phe Gly Leu
Val Cys Ser Ala Thr Asp Thr Leu Thr Ser Gln Pro
Val Ala Ile Lys Lys Ile Met Lys Pro Phe Ser Thr
Ala Val Leu Ala Lys Arg Thr Tyr Arg Glu Leu Lys
Leu Leu Lys His Leu Arg His Glu Asn Leu Ile Cys
Leu Gln Asp Ile Phe Leu Ser Pro Leu Glu Asp Ile
Tyr Phe Val Thr Glu Leu Gln Gly Thr Asp Leu His
Arg Leu Leu Gln Thr Arg Pro Leu Glu Lys Gln Phe
Val Gln Tyr Phe Leu Tyr Gln Ile Leu Arg Gly Leu
Lys Tyr Val His Ser Ala Gly Val Ile His Arg Asp
Leu Lys Pro Ser Asn Ile Leu Ile Asn Glu Asn Cys
Asp Leu Lys Ile Cys Asp Phe Gly Leu Ala Arg Ile
Gln Asp Pro Gln Met Thr Gly Tyr Val Ser Thr Arg
Tyr Tyr Arg Ala Pro Glu Ile Met Leu Thr Trp Gln
Lys Tyr Asp Val Glu Val Asp Ile Trp Ser Ala Gly
Cys Ile Phe Ala Glu Met Ile Glu Gly Lys Pro Leu
Phe Pro Gly Lys Asp His Val His Gln Phe Ser Ile
Ile Thr Asp Leu Leu Gly Ser Pro Pro Lys Asp Val
Ile Asn Thr Ile Cys Ser Glu Asn Thr Leu Lys Phe
Val Thr Ser Leu Pro His Arg Asp Pro Ile Pro Phe
Ser Glu Arg Phe Lys Thr Val Glu Pro Asp Ala Val
Asp Leu Leu Glu Lys Met Leu Val Phe Asp Pro Lys
Lys Arg Ile Thr Ala Ala Asp Ala Leu Ala His Pro
Tyr Ser Ala Pro Tyr His Asp Pro Thr Asp Glu Pro
Val Ala Asp Ala Lys Phe Asp Trp His Phe Asn Asp
Ala Asp Leu Pro Val Asp Thr Trp Arg Val Met Met
Tyr Ser Glu Ile Leu Asp Phe His Lys Ile Gly Gly
Ser Asp Gly Gln Ile Asp Ile Ser Ala Thr Phe Asp
Asp Gln Val Ala Ala Ala Thr Ala Ala Ala Ala Gln
Ala Gln Ala Gln Ala Gln Ala Gln Val Gln Leu Asn
Met Ala Ala His Ser His Asn Gly Ala Gly Thr Thr
Gly Asn Asp His Ser Asp Ile Ala Gly Gly Asn Lys
Val Ser Asp His Val Ala Ala Asn Asp Thr Ile Thr
Asp Tyr Gly Asn Gln Ala Ile Gln Tyr Ala Asn Glu
Phe Gln Gln,

or a polypeptide having at least 80%, 82%, 85%, 86%, 88%, 90%, 92%, 94%, 95%, 97%, 98% or 99% amino acid sequence identity thereto. An exemplary nucleotide sequence encoding HOG1 is

(SEQ ID NO: 7)
atgaccacta acgaggaatt cattaggaca cagatattcg
gtacagtttt cgagatcaca aatagataca atgatttaaa
ccccgttggg atgggggcat ttgggttggt ttgctcagcc
acggacactt tgacatctca gccagttgcc attaagaaaa
tcatgaaacc tttttccact gcagtgctgg ccaaaaggac
atatcgtgaa ctaaaactac taaaacatct aagacacgag
aacttgattt gccttcagga catatttctt tctccattgg
aagatatata ttttgtcacg gaattacaag gaacagattt
acatagactc ttgcaaacaa gacccttgga aaagcaattt
gttcagtatt tcctatacca aattctaagg ggtttaaaat
acgttcactc cgcgggcgtc attcatagag atttgaaacc
gagcaacatt ctgattaatg aaaactgtga tttgaagatt
tgcgatttcg gtctagcaag aattcaagac cctcaaatga
caggctatgt ttccactaga tactacaggg cacctgaaat
catgctaacg tggcaaaaat atgacgtcga ggtcgacatt
tggtccgctg gttgtatttt tgccgaaatg attgaaggta
agcctttgtt ccctgggaaa gatcatgttc accaattttc
gatcatcact gacttgttgg gatctccgcc aaaggatgtg
ataaatacta tttgttccga aaatactcta aaatttgtta
cttcgttacc acacagagat ccaattccat tttctgaaag
atttaaaaca gtcgaacctg atgccgtaga ccttttggaa
aaaatgctgg tttttgatcc taagaagaga atcactgcgg
cggatgcctt ggctcatcct tattcggctc cttaccacga
tccaacggat gaaccagtag ccgatgccaa gttcgattgg
cactttaatg acgctgatct gcctgtcgat acctggcgtg
ttatgatgta ctcagaaatc ctagacttcc ataagattgg
tggcagtgat ggacagattg atatatctgc cacgtttgat
gaccaagttg ctgcagccac cgctgccgcg gcgcaggcac
aggctcaggc tcaggctcaa gttcagttaa acatggctgc
gcattcgcat aatggcgctg gcactactgg aaatgatcac
tcagatatag ctggtggaaa caaagtcagc gatcatgtag
ctgcaaatga caccattacg gactacggta accaggccat
acagtacgct aatgagttcc aacagtaa,

or a nucleotide sequence having at least 80%, 82%, 85%, 86%, 88%, 90%, 92%, 94%, 95%, 97%, 98% or 99% nucleic acid sequence identity thereto.

Exemplary sequences to decrease or disable expression of Cox15 in the host cells of the disclosure include but are not limited to sequences that encode

(SEQ ID NO: 6)
mlfrnievgr qaaklltrts srlawqsiga srnistirqq
irktqlynfk ktvsirpfsl sspvfkphva sesnpiesrl
ktsknvaywl igtsglvfgi vvlggltrlt esglsitewk
pvtgtlppmn qkeweeefik ykespefkll nshidldefk
fiffmewihr lwgraigavf ilpavyfavs kktsghvnkr
lfglagllgl qgfvgwwmvk sgldqeqlda rkskptvsqy
rltthlgtaf flymgmlwtg leilreckwi knpvqaislf
kkldnpaigp mrkislalla vsfltamsgg mvagldagwv
yntwpkmger wfpssrelmd enfcrredkk dlwwrnllen
pvtvqlvhrt cayvaftsvl aahmyaikkk aviprnamts
lhvmmgvvtl qatlgiltil ylvpislasi hqagalallt
sslvfasqlr kprapmrnvi itlphsskvt sgkilseask
laskpl,

or a polypeptide having at least 80%, 82%, 85%, 86%, 88%, 90%, 92%, 94%, 95%, 97%, 98% or 99% amino acid sequence identity thereto.

Exemplary sequences to decrease or disable expression of IRA1 in the host cells of the disclosure include but are not limited to sequences such as

(SEQ ID NO: 8)
cacccgtcct gtggatcatc ttttgccctg caaatagagc ttcaaactta acattcttct
tcagcatata acatacaaca agattaaggc tctttctaaa atgaatcaaa gcgatccgca
agacaaaaaa aatttcccaa tggaatactc tttgaccaag catctttttt tttgataggc
ttctacttgt tcttcccata gaatctaatt tgaaaacata tgctgatgtg gaggcagatt
cagttttcaa ttcgtgtcgg tccatcattt tgaatatagc catcactaag gacttgaacc
cgattatcga aaacacatta ggtttaattg acttgattgt gcaagatgaa gaaattacgt
ctgacaatat tacagatgat attgcccatt ctatattggt tcttttgaga ttactgagtg
atgtttttga gtattactgg gatcaaaaca atgacttcaa gaaaattaga aacgataatt
acaaaccggg attttcaagt cacaggccaa acttccatac atctaggcca aagcacacga
gaatcaatcc agctttggcg acgatgttac tatgtaaaat ttctaagctg aagttcaata
caagaacttt aaaggtttta cagaacatgt ctcaccatct ttctggcagc gctactatct
caaaatcgag tattttaccc gattcacagg aatttttaca aaagagaaac tatccagcat
ataccgagaa aatagattta acaatagatt atatccagag attcatatct gcttccaatc
atgttgaatt cacaaagtgt gtcaaaacaa aagttgttgc acctttattg atatcacaca
cctcaaccga attgggcgta gtaaaccact tggatttatt tggttgtgag tatttgactg
ataagaatct gctagcatat ctggacatac tacaacacct gtcaagttac atgaagagga
ccatttttca ttcgcttttg ttatattatg cttccaaagc ttttttattt tggataatgg
caaggccaaa agaatacgtc aaaatttata acaatctaat atcatcagat tataatagtc
cgtcttcttc atctgataat ggtggttcga ataattctga taaaacgtct atatcccaac
tagtctcact gttattcgat gacgtttatt ccacttttag tggatcatca ttattaacaa
atgtcaataa tgaccaccac taccatcttc atcattcatc ttcttcatca aagacgacca
acactaatag tccaaactct atatcaaaaa cgtcgataaa gcagtcgagt gtgaatgctt
ctggcaatgt ttctccgtct cagttttcta ctgggaatga tgcatcgcct acttccccta
tggcatcatt gagttcaccc ttaaacacga acatcctagg gtatccgtta tctccaatca
cttcaacact aggacaggcg aatacttcca catcgactac ggctgcaact accaaaacag
atgcagatac gccctctact atgaatacta acaacaataa taacaataac aacagcgcta
atcttaataa tattccacaa cgcatatttt ccttagatga catttcatcc tttaactcga
gtagaaaatc actcaatcta gatgatagta actccttgtt tctttgggat acttctcagc
attctaatgc atcgatgaca aatacaaata tgcatgcagg agttaataat tctcagtctc
agaacgatca gtcttcttta aactatatgg aaaatattat ggagctgtat tccaactata
ccggatcaga actatcctcc catactgcca tattaaggtt tttggtggtt ctgaccttat
tagacagtga agtatatgat gagatgaact caaattcgta tagaaaaatt tcggaaccga
taatgaatat taatccgaag gactctaata cttcaagttg gggctcagca tccaaaaacc
caagtatcag gcacctcacc catggcttaa aaaaacttac tttacagcaa ggcaggaaac
gtaacgttaa atttttgaca tatttgatta gaaatttgaa tgggggacag ttcgtttcag
atgtttcctt gattgactct atcaggtcca ttctattctt aatgacaatg acgtcttcta
tatcccaaat cgattcaaat attgcttctg ttattttttc gaagagattc tacaacttgt
tgggtcaaaa tttagaggtc ggcaccaatt ggaattctgc cactgcaaat acttttattt
ctcattgtgt tgaaaggaat ccccttacac ataggcgttt acaattagag ttttttgcaa
gcggtttaca gctggattct gatttatttt taagacattt acaactggaa aaagaactca
atcacataga ccttcccaaa atatcgttat acactgaagg atttagggta ttttttcacc
tagtaagcac caaaaaactt catgaggata ttgcagaaaa aacctcctct gtgttaaaga
gacttttctg cataattgct gatattttgt tgaaagcaac gccttatttt gacgataatg
taaccaagat tattgcttct atattggatg ggcatatttt agatcaattt gacgctgcgc
gaacactttc taatgatgat catgtcagtt ttgatgctgc cacaagcgtt tacactgagc
caaccgaaat tattcataac tcatcggatg cctcgttagt ctcttcactt tcccaatcac
ccttatcaat taactcagga agcaatatca ccaatacgcg cacctgggat attcaatcaa
tcttaccaac cttatcgaac agatcaagtg cttctgattt gagcttgtct aacattttga
ctaatccgtt ggaggcacaa caaaataata atgcaaactt gttagcccat agattatctg
gggttcctac tactaagaga tacgcttcac cgaacgactc tgaaagatca cgacaaagtc
catattcttc tccgccgcaa ttgcaacaaa gtgacttgcc ttctccgctc tcagtcctct
cgtcaagtgc aggattttct tctaatcatt cgattacggc aaccccaact attttgaaaa
acatcaaatc tccaaaacca aacaaaacaa aaaaaattgc tgatgataaa caattgaaac
agccttctta ttcaagagta atactgagtg acaatgatga agcaagaaag attatgatga
acattttcag cattttcaaa agaatgacca actggtttat acgcccagat gctaatacag
aattcccgaa gacttttacg gatattataa aaccactttt tgtctctata ttggattcta
atcaaagact acaagttaca gcgcgggctt ttattgaaat cccattaagt tatatagcta
cttttgaaga cattgataat gatcttgacc caagagtact gaatgaccat tatttgttat
gtacatatgc cgttactttg tttgcttcat cattgtttga tttgaagtta gaaaatgcga
agagagagat gctactagac attattgtta aatttcaacg agtccgttct tatttatcaa
atttagcaga aaaacacaac ctagtccagg caataattac gacggagagg ttgacgctgc
cattattagt tggtgctgta ggaagtggaa ttttcatttc attatactgc agtcgtggaa
atacgccacg cttaataaaa atttcatgtt gtgaatttct acgatccttg agattttatc
aaaaatacgt aggcgctttg gatcaatatt ccatttacaa tattgatttc atagatgcta
tggcccagga caatttcact gcctcaggat cagtggcttt gcaacggcgt ctaagaaata
atattttaac ttatatcaaa ggatccgact caatcctttt ggattcaatg gacgtgattt
acaagaagtg gttttacttc agctgttcga aatcagttac gcaagaggaa ctagtagatt
ttagaagctt ggcaggcatt ctagcttcta tgtcaggtat tctgtctgat atgcaagagt
tggaaaaaag caagagcgct ccagataatg aaggagacag cttatcattt gagtcacgaa
atcccgctta tgaggtgcac aaaagtctta aactcgagtt aacgaaaaaa atgaatttct
ttatttcaaa acaatgtcaa tggttgaata atccaaatct attaacaaga gaaaattcga
gagatatatt aagtattgag ttgcatcctc tatcttttaa cttattgttt aacaacctag
gactgaaaat agatgaactg atgtcaattg atctttcaaa gtcacatgag gattcatcgt
ttgttttact agagcagata ataattataa taagaactat actaaagagg gatgatgatg
aaaagataat gctactcttt tcgacggact tgcttgatgc ggtcgataag ttgatcgaaa
tagtggagaa aatttccatc aagtcctcca aatattataa gggaattatc caaatgtcga
aaatgtttag agcatttgag cactctgaaa agaacctggg catttcaaat catttccatt
taaagaataa atggttgaag ttagttattg gttggttcaa actatctatt aataaggatt
atgattttga aaacctgtca agaccattaa gggaaatgga tttgcagaaa agggacgaag
attttttgta tatcgacact tctattgaat ctgcaaaagc attggcttac ctaacacata
atgttccttt agaaataccg ccttcaagct caaaagaaga ttggaacaga tcttctacag
tatcatttgg caatcacttt actattttgt taaaaggtct ggagaaaagc gcggacctga
atcagtttcc agtttcatta aggcataaga tcagtatact taatgaaaat gtaataatag
cgctaacgaa cttatctaat gccaatgtca acgtttcttt aaaattcact ttaccaatgg
gttattctcc aaataaagat atcagaatcg cctttttaag agttttcatc gacatagtaa
ccaactatcc agttaaccct gagaaacatg aaatggataa aatgctagct atagacgact
tcctgaaata tataatcaag aacccaatat tagcattttt cggaagttta gcgtgttctc
ctgctgatgt tgatttatat gctggtggat tcttaaacgc ctttgacact agaaatgcgt
ctcatatcct tgttactgag ctccttaaac aagaaatcaa acgggccgca agatcagacg
atattctcag aagaaatagt tgtgcaacaa gggctttgtc actttacact agatctagag
gtaacaaata tttgataaaa actttgagac ccgttttgca agggatagtg gataacaagg
agtcttttga aattgataag atgaaaccag gatccgaaaa ctccgaaaag atgttagact
tatttgagaa gtacatgaca agattaattg acgcaattac aagttctatt gatgatttcc
caatagaatt agttgatatc tgtaaaacaa tttacaatgc tgctagtgta aattttccag
aatacgcata tattgccgtt gggtcattcg ttttcttgag gtttatcggg cctgctttag
ttagtcctga ttcggaaaat atcattattg ttacgcacgc ccatgacaga aagcccttta
ttacactagc taaagttatt caaagtttag ctaatggcag ggaaaatata ttcaagaaag
atatcttagt ttcaaaagaa gagtttttga aaacctgtag tgataaaata ttcaattttt
tgtctgaatt gtgcaagata ccgactaaca atttcaccgt caatgtaaga gaagatccga
caccaataag ctttgactac tcatttttgc ataaattctt ttacctcaat gagtttacca
taagaaaaga aattattaat gaatccaaat taccagggga gttcagcttt ttgaaaaata
ctgttatgct caacgacaaa attcttggtg tattgggaca acctagcatg gaaataaaaa
atgaaattcc tccttttgta gtcgagaatc gggaaaaata tccttcattg tatgaattca
tgagtcgcta tgccttcaaa aaagtggaca tgaaagaaga agaagaggat aatgcgccat
ttgtacatga agcaatgaca ttggatggca tacaaatcat tgtcgtaact tttaccaatt
gcgagtacaa taattttgta atggactcac tggtctataa agttctgcag atatatgcaa
gaatgtggtg ctctaaacat tatgtagtta tcgattgtac caccttttat gggggtaagg
ctaatttcca aaaattgact actctatttt tcagtttgat accagagcaa gcatcaagta
attgtatggg atgttattac ttcaacgtca acaaatcatt tatggaccaa tgggcctcat
catatactgt agaaaatccg tacttggtca ctacaattcc ccgttgtttc atcaacagca
atactgacca aagtttgata aagtccttag gattgagtgg taggagtttg gaagttttga
aagatgtaag agttactttg catgatatta ccctttatga caaggaaaaa aagaagtttt
gtcccgtgtc cttgaagata ggaaacaaat acttccaagt tttacatgag attccgcagt
tgtacaaggt taccgtatca aacaggacat tcagcatcaa attcaacaat gtttacaaga
tatcaaattt aatttcagtc gatgtctcta acaccacagg cgtttcctcg gaatttacgt
taagtcttga taatgaagaa aagttggtat tttgcagtcc gaagtaccta gaaattgtga
aaatgtttta ttatgcccag ttaaagatgg aagaagactt tggtacggat ttttcgaacg
atatttcatt ttcaacatcc tcttcagcag ttaatgcttc ttactgcaat gttaaagaag
ttggtgaaat tatatcacat ttgtcattgg tgatccttgt aggtttattc aatgaggatg
atctcgtcaa aaacatatca tacaaccttc tcgtggcaac gcaagaagca tttaatttag
attttgggac aaggcttcac aaatccccag agacatatgt acccgatgat accaccacgt
tcttggccct aattttcaag gctttttcag aatcttcaac ggaactaact ccatatatat
ggaaatatat gctggatggc cttgaaaacg acgtgattcc tcaagaacat attcctacgg
ttgtctgttc attgtcatac tgggtaccaa acttatatga acatgtatat ttggcaaatg
acgaagaggg accagaggcg atttcacgta taatctatag cttaatcagg ttgacggtca
aagagccaaa tttcacgaca gcttaccttc aacagatttg gtttttactg gcattggatg
gtcgtctcac gaacgtgata gttgaagaaa tagtaagtca tgcgctggat agagattcag
aaaacagaga ctggatgaaa gctgtgtcaa tactaaccag ttttccaacg acagagattg
cttgtcaggt aatagagaag ctaataaata tgatcaaatc ttttctacct tctctagcag
ttgaggcttc cgcacacagt tggtctgagc ttactatttt atcaaaaatt agtgtgtcaa
ttttctttga atcaccctta ctttcccaga tgtatttacc ggagattctt ttcgctgtgt
ctctgttaat tgatgtcggt ccttcggaaa taagagtctc attgtacgag ttgttgatga
atgtttgtca ttctttaacc aacaatgagt ccttacctga aaggaatagg aaaaatttgg
atatcgtctg tgcaacattc gcacgtcaaa agttgaactt tatctccggt tttagccaag
aaaaaggtag agttttacca aattttgccg cttcctcctt ctccagtaaa ttcggaacat
tagatctctt cactaaaaac attatgctat tgatggaata tggttctatt tcagagggtg
cacaatggga ggcaaaatat aagaaatatt tgatggatgc gatttttggc catcggtcgt
tcttctctgc gagagctatg atgattctag gtataatgag taagtcgcac acgtcccttt
tcctttgtaa agaactttta gttgaaacca tgaaggtctt cgcagagcca gttgtggatg
atgaacaaat gttcatcatt atagctcatg tctttactta cagcaaaatt gtcgaagggt
tagatccttc ttcagaatta atgaaagagc tattttggct tgctacaata tgtgttgaat
cccctcatcc tttactcttt gaaggtggtc tcctgttcat ggtaaattgt ttgaagcgac
tgtacacggt ccatcttcaa cttggattcg atggcaaatc gctagccaaa aaattaatgg
aatctagaaa ttttgctgct acgcttttgg ctaagttaga gtcatacaat ggatgcatat
ggaacgaaga taattttcct catattattt taggtttcat tgcaaacggt ttatccattc
ctgtcgtaaa aggagccgca ttagattgtc tacaggccct tttcaagaat acatattacg
aaagaaagtc caacccaaaa tcctccgatt atctttgtta ccttttctta ctccatttgg
tcttaagtcc tgaacaactt tctaccttgt tacttgaagt cggcttcgaa gatgaactgg
tacctttaaa taatacacta aaagtgccac ttactttgat caactggcta agttcagact
cagataaatc taatatagtc ttataccaag gagcactttt gtttagctgt gttatgtcag
acgaaccatg taaattccgt tttgctctat tgatgaggta tttgctcaaa gtcaacccta
tttgtgtatt caggttctat acgctgacta gaaaggaatt caggaggtta tcaaccctag
aacaatcatc tgaagcggtt gctgtctctt ttgaattgat tgggatgctt gttacacaca
gtgagtttaa ttacctagag gaatttaatg atgaaatggt cgaactttta aaaaagagag
gcttgtcagt tgtgaagcct ctggatattt ttgatcagga acatatagaa aagttaaaag
gagagggtga acatcaagtg gcaatttatg agagaaaaag attagcaaca atgatactgg
caagaatgtc gtgctcctaa ttttaaatta gagtattgca gtgattatat acgtttttcc
ttataattat attttgtttt atatttgttg gattgtagat tttaaattaa atgaaacgta
gagatttgtg taaagtataa tgattgcaag gttaagagtg aaacattttt agagcactac
taactagcat tttccaaggt ggaaagatcc gaaaaagcac gattaccgga aatccaactg
ttgaccattt ccaaacttgc ctccggttga tc,

or a nucleotide sequence having at least 80%, 82%, 85%, 86%, 88%, 90%, 92%, 94%, 95%, 97%, 98% or 99% nucleic acid sequence identity thereto.

Exemplary disabling mutations include but are not limited to:

| | Functional Gene | Nucleotide | Amino Acid |
|---|---|---|---|
| Gene | Annotation | Difference | Difference |
| ISU1 | Fe—S cluster assembly | C412T | H138Y |
| HOG1 | MAP kinase signaling | A844del | M282frame- |
| | | | shift⁴ |
| GRE3 | Aldose reductase | G136A⁵ | A46T |
| IRA2 | Inhibitor of RAS | G8782T | E2928Stop |

The invention will be described by the following non-limiting examples.

### EXAMPLE

Corn stover biomass, the most common source of carbon for production of bioethanol, is about 35% glucose polymer and about 19% xylose polymer. Current methods of bioethanol production can only access the glucose, as fermentation of xylose to ethanol is too slow and inefficient to be economically viable. Opening up xylose as a viable carbon source would increase the total amount of fuel available per unit of feedstock, as well as enabling the usage of feedstocks with a higher ratio of xylose to glucose. It has been a goal of researchers and industry for some time to generate large amounts of bioethanol from non-food feedstocks including switchgrass, but the only way the economics as using switchgrass as an ethanol feedstock work is if xylose as well as glucose can be efficiently fermented.

In this study, genetic changes were identified that enable S. cerevisiae to ferment xylose into ethanol at high flux, such that it can ferment xylose aerobically without the need for respiration, a hallmark of the Crabtree-Warburg Effect for glucose. Respiration was blocked in a xylose-consuming S. cerevisiae strain by deleting COX15, which encodes a mitochondrial inner membrane protein involved in the synthesis of heme A, an essential prosthetic group for cytochrome c (Barros et al., 2001; Glerum et al., 1997). This respiration-deficient mutant strain was then subjected to aerobic ALE with xylose as the primary carbon source. Evolved clones selected for their abilities to ferment xylose into ethanol aerobically without respiration were then genome-sequenced to determine the causative genetic changes. By reengineering two genetic changes found in the evolved strains into the original parent strain, a strain was generated with increased xylose fermentation aerobically in the absence of respiration. Importantly, this reengineered strain fermented xylose into ethanol at faster rates anaerobically in both lab medium and lignocellulosic hydrolysate. Finally, these genetic changes increased xylose conversion in another strain background, suggesting that the combination of genetic modifications could be incorporated into existing industrial S. cerevisiae strains

### Materials and Methods

### Media

Standard undefined yeast lab media were prepared as previously described (Sherman, 2002). Briefly, media for culturing yeast contained 10 g/L yeast extract, 20 g/L peptone (YP), and various carbon source concentrations (X=20-30 g/L xylose, D=20 g/L dextrose/glucose, Gal=20 g/L galactose, Gly=20 g/L glycerol, EtOH=15 g/L ethanol). Solid media also contained 2.5% agar and 200 μg/mL Geneticin (US Biological, Swampscott, MA), 200 μg/mL Hygromycin B (US Biological, Swampscott, MA), 100 μg/mL Nourseothricin (Jena Bioscience, Jena, Germany), or 200 μg/mL Zeocin (Thermo Fisher Scientific, Waltham, MA) as needed. AFEX-pretreated switchgrass hydrolysate (ASGH) was prepared as described elsewhere (Zhang et al., 2020).

### Construction of Engineered S. cerevisiae Strains

Yeast strains used in this study are described in Table 2. Deletions of COX15, TAL1 , xylA, XYL3, TKL1, GRE3, IRA2, HOG1, and ISU1 were performed by integration of polymerase chain reaction (PCR) products generated from LoxP-KanMX-LoxP (pUG6) or LoxP-HphMX-LoxP (pUG75) plasmid templates (Guldener et al., 1996) and primers containing 40-60 bp of homology sequences flanking the targeted gene (Parreiras et al., 2014). PCR products were purified and transformed (Gietz and Schiestl, 2007) into the appropriate strains. Cre recombinase-mediated excision of LoxP-flanked antibiotic markers was carried out as published elsewhere (Guldener et al., 1996). Complementation of the cox15Δ mutation was performed with the modified pRS416 plasmid (Christianson et al., 1992) Christianson et al., 1992) with the URA3 auxotrophic marker replaced with the HphMX marker (pRSCENHygMX) and containing the COX15 promoter, open reading frame (ORF), and terminator. Insertion of additional copies of TAL1 -xylA-XYL3 expression cassette (XYL cassette) and TKL1 promoter, terminator, and ORF were performed using CRISPR/Cas9-mediated genome editing as previously described (Higgins et al., 2018) with some modifications. First, a KanMX antibiotic marker was used to replace the GRE3 ORF (gre3Δ:: LoxP-KanMX) or inserted between nucleotides 199269 and 199270 of Chrl (ChrI¹⁹⁹²⁶⁹) by homologous recombination in the desired strain. ChrI¹⁹⁹²⁶⁹ is the site of pseudogene, which has been previously used as a location for transgene insertion (Hittinger and Carroll, 2007). Selected strains were then transformed with the pXIPHOS plasmid (Higgins et al., 2018) to express Cas9 and an sgRNA sequence (ATGAAGGAGAAAACTCACCG; SEQ ID NO:1), which was designed by CRISpy-pop (Stoneman et al., 2020) to target KanMX. The XYL cassette and TKL1 PCR products containing 50-60 bp of flanking sequences to GRE3 and the ChrI locus, respectively, were co-transformed at 20-fold molar excess as repair templates. Transformed colonies were restreaked onto YPD twice to cure the pXIPHOS plasmid, followed by PCR and Sanger sequencing verification of TAL1 -xylA-XYL3 and TKL1 insertions. All strains were confirmed for gene deletion and antibiotic marker excision by PCR with independent outside flanking primers. Sanger sequencing of PCR products and DNA plasmids was performed by University of Wisconsin-Madison Biotechnology Center.

### Adaptive Laboratory Evolution (ALE)

ALE for selection of clones that ferment xylose aerobically without respiration was performed using the GLBRCY583 (Y583) strain, which lacks COX15. Three separate flasks containing 30 mL YP medium with 3% xylose and 0.05% glucose were inoculated with Y583 to optical density at 600 nm wavelength (OD₆₀₀) of 0.2 and shaken at 250 rpm and 30° C. When the cell densities of each flask reached OD₆₀₀=2-6, the cultures were subcultured with fresh medium to OD₆₀₀=0.2. Medium for the second passage included 0.1% glucose, whereas all subsequent passages lacked glucose. Between 6-8 passages for each culture were performed until the maximum cell densities were reached within 48 h and all xylose was consumed from the medium as determined by high performance liquid chromatography (HPLC) and refractive index detection (RID) (Schwalbach et al., 2012). Two out of the three ALE cultures that displayed significant growth on xylose-only medium were plated onto YPX+Hygromycin B agar plates. Isolated colonies from the two evolved flasks were tested for growth on xylose aerobically in flask experiments described below. OD₆₀₀ measurements were performed using 1-cm pathlength cuvettes and a Beckman Coulter DU720 spectrophotometer. Single clones (Evo1 and Evo2) from the two independently-evolved flasks with the fastest cell growth and xylose consumption rates were selected for further study.

### Genomic DNA Library Preparation and Sequence Analysis

Genomic DNA preparation and Illumina sequencing were performed as described previously (Sato et al., 2016) with some modifications. DNA was submitted to the University of Wisconsin-Madison Biotechnology Center. DNA concentration was verified using the Qubit® dsDNA HS Assay Kit (Life Technologies, Grand Island, NY). Samples were prepared according the TruSeq Nano DNA LT Library Prep Kit (Illumina Inc., San Diego, California, USA) with minor modifications. Samples were sheared using a Covaris M220 Ultrasonicator (Covaris Inc, Woburn, MA, USA), and were size selected for an average insert size of 550 bp using SPRI bead-based size exclusion. Quality and quantity of the finished libraries were assessed using an Agilent DNA 1000 chip and Qubit® dsDNA HS Assay Kit, respectively. Libraries were standardized to 2 nM.

Cluster generation was performed using the Illumina PE Cluster Kits v4 and the Illumina cBot. Paired-end, 125 bp sequencing was performed, using v4 SBS chemistry on an Illumina HiSeq2500 sequencer. Images were analyzed using the Illumina Pipeline, version 1.8.2. All DNA sequencing reads have been deposited in the NCBI SRA under Bio-Project PRJNA279877. Paired-end reads were mapped to GLBRCY22-3 (GCA_001634645.1) (McIlwain et al., 2016) using Bwa-mem 0.7.12-r1039 (Li, 2013). Genomic variants were identified using GATK (v3.4) (McKenna et al., 2010) with duplicate marking and indel realignment. Variants were called using Haplotype Caller without input of known sites/variants. GATK variant filtration was done using the GATK-recommended criteria: QD<2, FS>60, and MQ<40. Coverage analysis was performed by summing unique counts in 500-bp non-overlapping windows using custom Python and R scripts and visualized using the sppIDer pipeline (Langdon et al., 2018).

### Cell Culturing and Phenotypic Growth Analysis

Aerobic tube and anaerobic flask fermentation assays were performed as previously described (Parreiras et al., 2014) with some modifications. For aerobic and anaerobic growth assays, inoculum cultures were started from single colonies grown in YPD medium overnight and passaged to OD₆₀₀=0.3. Log phase cultures were then diluted to OD₆₀₀=0.1 in the appropriate medium at the start of the assay. Yeast cultures were grown in culture tubes containing 10 mL of medium shaken at 250 rpm at 30° C. or in 30 mL of medium stirred with a magnetic stir bar in flasks placed in an anaerobic chamber (Coy Laboratory Products Inc. Grass Lake, MI), which was maintained with 2-5% H₂, 2-5% CO₂ and 90-95% N₂. For COX15 complementation experiments, cox15Δ strains transformed with the pRSCENHygMX (Empty vector) or pRSCENHygMX-COX15 plasmid were cultured in medium containing 200 μg/mL Hygromycin B. For cell culture experiments using Antimycin A, yeast cells were grown to log phase in YPD medium aerobically and then shifted into tubes containing 10 mL fresh YPD or YPX medium treated with 10 μL DMSO or 10 μL of 0.5 mg/mL Antimycin A in DMSO (0.5 μg/mL final concentration, A8674, Sigma-Aldrich, St. Louis, MO), and incubated at 30° C. with shaking. For aerobic cell growth assay using glucosamine, yeast cells were grown to log phase in YPD medium aerobically and then shifted into tubes containing 10 mL fresh YPD or YPD medium treated with 250 μL of 200 mg/mL D-(+)-glucosamine hydrochloride (5 mg/mL final concentration, G4875, Sigma-Aldrich), and incubated at 30° C. with shaking.

For anaerobic bioreactor experiments, fermentations were conducted in 0.25 L Minibio bioreactors (Applikon Biotechnology Inc., Foster City, CA) containing 100 mL of 7% glucan-loading ASGH. Prior to fermentation, hydrolysates were adjusted to pH 5.0 and filtered through a 0.2 μm filter to remove precipitates and to ensure sterility. After transfer to the fermentation vessel, hydrolysates were sparged with 100% N₂ at the flow rate of ˜20 mL/min overnight before the inoculation. S. cerevisiae strains were grown to early stationary phase in YPD aerobically and then diluted OD₆₀₀=0.2 in fresh YPD for ˜10 h. Cultures were then centrifuged at 3,000×g for 5 min, the cell pellets were resuspended into ˜10 mL of hydrolysate from the pre-sparged vessels and then inoculated back into each bioreactor to a starting OD₆₀₀ of 0.5. Fermentations were conducted at 30° C. with continuous stirring (500 rpm) and sparged at ˜20 mL/min with 100% N₂. During the fermentation, pH was maintained at 5.0 by automated addition of 5% NaOH. Cell density measurements were blanked with OD₆₀₀ measurements from uninoculated hydrolysate diluted 1:10 or 1:25 with water. Extracellular glucose, xylose, ethanol, glycerol, and galactose concentrations for all experiments were determined by HPLC-RID. Calculations for xylose consumption and ethanol production rates and yields from anaerobic bioreactor fermentations were determined for the time period after all glucose was depleted and as described elsewhere (Sato et al., 2016).

### Results

### Evolved Yeast Mutants Convert Xylose Into Ethanol Aerobically in the Absence of Respiration

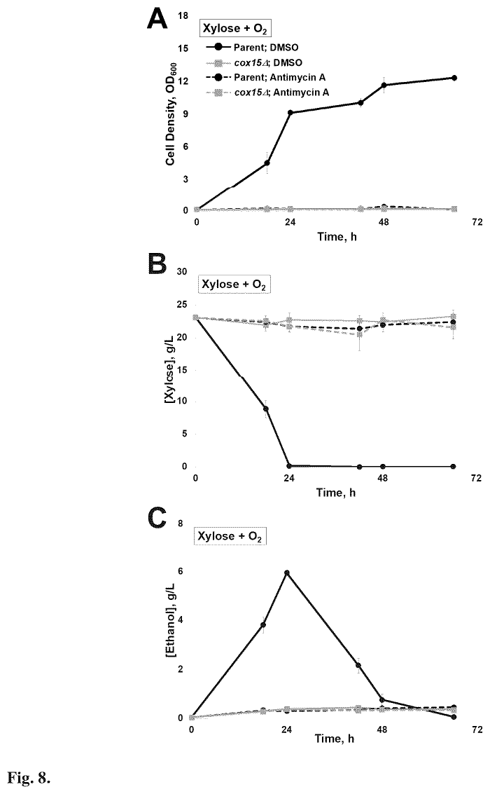

Previously, a S. cerevisiae strain was engineered to express single copies of xylose isomerase (xylA from Clostridium phytofermentans) and xylulokinase (XYL3 from Scheffersomyces stipitis), as well as an additional copy of the native transaldolase TAL1 by genomic insertion of a DNA cassette (henceforth called the “XYL cassette”). Informed by ALE (Sato et al., 2016), deletion mutations were engineered in HOG1, ISU1, GRE3, and IRA2 to enable rapid conversion of xylose into ethanol anaerobically. Interestingly, this xylose-fermenting strain (hereafter referred to as the “Parent” strain) converted xylose into ethanol in the presence of oxygen, albeit worse than the theoretically expected ethanol yield from xylose. However, this Parent strain could not grow on and ferment xylose aerobically in the presence of Antimycin A (Sato et al., 2016), or with the cox15Δ mutation, which is essential for the function of Electron Transport Chain Complex IV (FIG. 8). Thus, the Parent strain requires respiration to grow on xylose in the presence of oxygen.

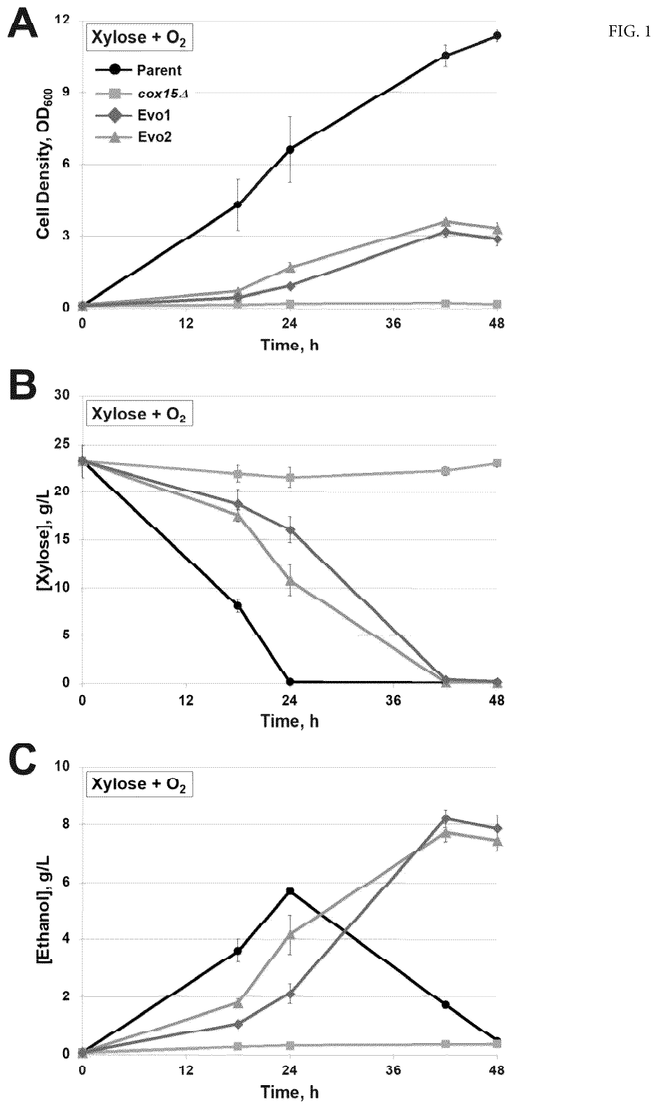

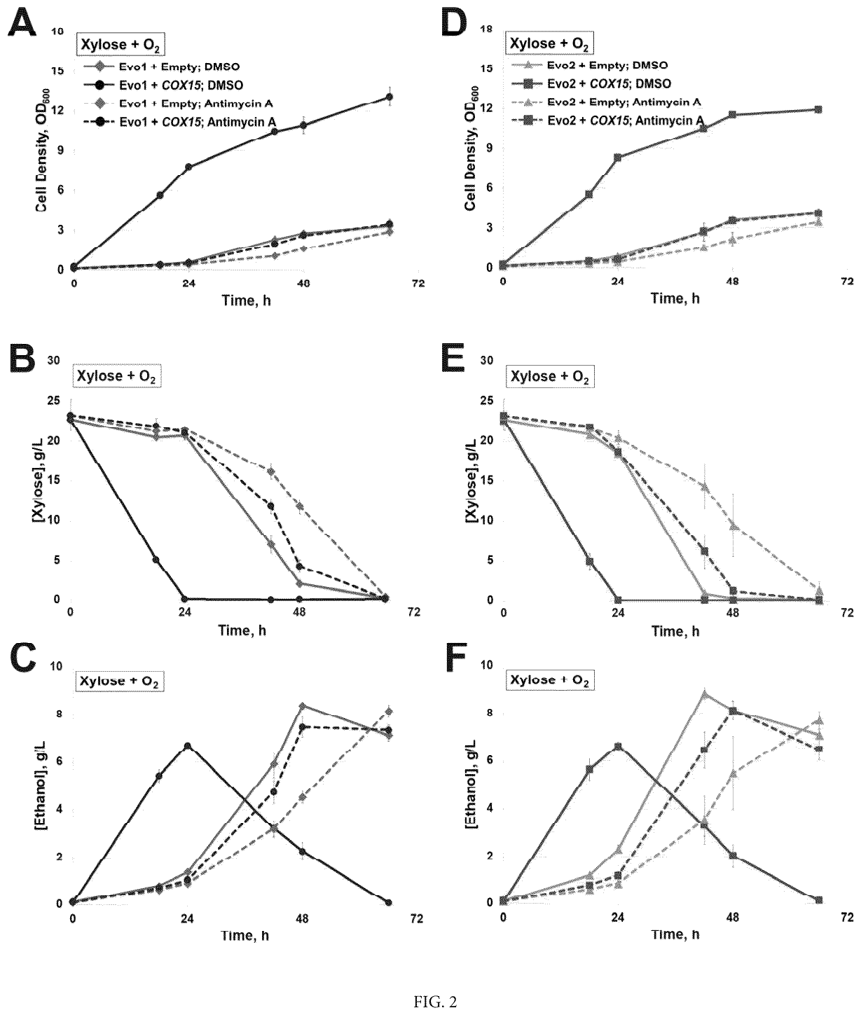

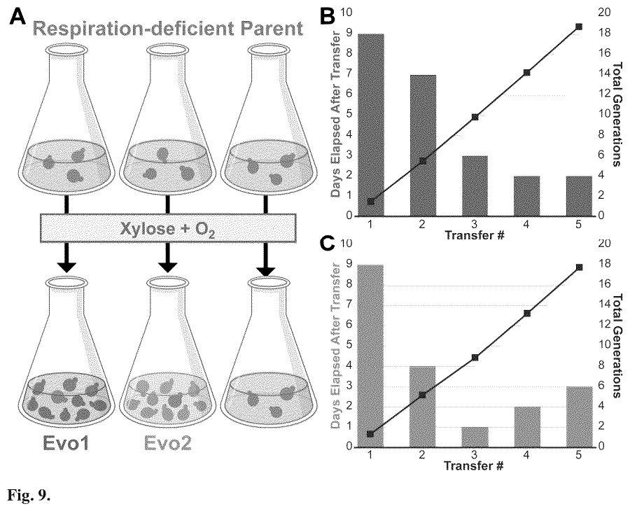

To increase the flux of xylose to ethanol without aerobic respiration, ALE was used to select for respiration-deficient mutants that grow aerobically on xylose. Specifically, the Parent strain with the cox15Δ mutation was evolved aerobically on xylose in three separate flasks (FIG. 8 (A)). Two out of the three flasks displayed significant growth during the third passage (FIG. 8 (B-C)). From each of these two flasks, independent clones (Evo1 and Evo2) were isolated and confirmed to grow on and ferment xylose into ethanol aerobically (FIG. 1). The evolved strains grew on and consumed xylose more slowly than the Parent strain, but they produced higher levels of ethanol, likely due to their inabilities to respire. It was confirmed that the Evo1 and Evo2 strains did not evolve the ability to respire without COX15 since both Evo1 and Evo2 strains did not grow on glycerol or galactose (FIG. 9). These results indicated that the evolved strains ferment xylose into ethanol aerobically without the requirement for respiration. A possible, albeit unlikely, mechanism for aerobic growth on xylose by the evolved strains could be from bypassing the need for Cox15p. To test this, the Evo1 and Evo2 strains were transformed with a low copy plasmid with or without COX15. The evolved strains expressing COX15 restored the ability to respire xylose and ethanol aerobically (FIG. 2). Then these strains were cultured in xylose aerobically with the addition of Antimycin A, which inhibits oxidative phosphorylation by the Electron Transport Chain Complex III. The evolved strains, with or without COX15, could grow on and ferment xylose into ethanol aerobically in the presence of Antimycin A (FIG. 2), which is a phenotype analogous to how Crabtree-Warburg-positive yeasts metabolize glucose. Together, these results suggest that the evolved strains can metabolize xylose into ethanol aerobically without the requirements for Complex III and IV activities.

### Evolved Mutations do not Bypass Glucose Repression

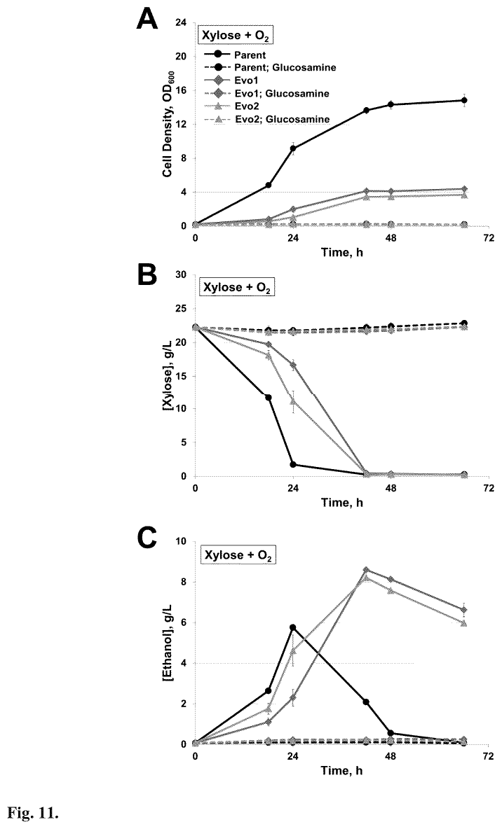

Another hallmark of Crabtree-Warburg-positive yeasts is glucose-mediated repression of metabolic pathways. In the presence of high levels of glucose, the Snflp pathway is inactivated to repress the expression of genes involved in gluconeogenesis, respiration, and non-fermentable carbon metabolism, leading Crabtree-Warburg-positive yeasts to metabolize glucose first (Kayikci and Nielsen, 2015). Extracellular glucose is also bound by the sensors Rtg2p and Snf3p, leading to a signaling cascade that relieves repression by the transcription factor Rtg1p (Johnston and Kim, 2005). These regulatory pathways likely lead to inefficient fermentation of other sugars, such as xylose, present in lignocellulosic hydrolysates into ethanol; thus, overcoming glucose repression is important for the biofuel industry. It was examined whether aerobic xylose fermentation by the evolved strains could be repressed by the non-metabolizable glucose analog, glucosamine. Aerobic growth and xylose consumption by both the Parent and the evolved strains were blocked in the presence of glucosamine (FIG. 11), which suggests that the fermentation of xylose into ethanol aerobically by the Evo1 and Evo2 strains requires genes and pathways that are repressed by glucose.

### Evolved Mutations Enhance the Rate of Anaerobic Xylose Fermentation

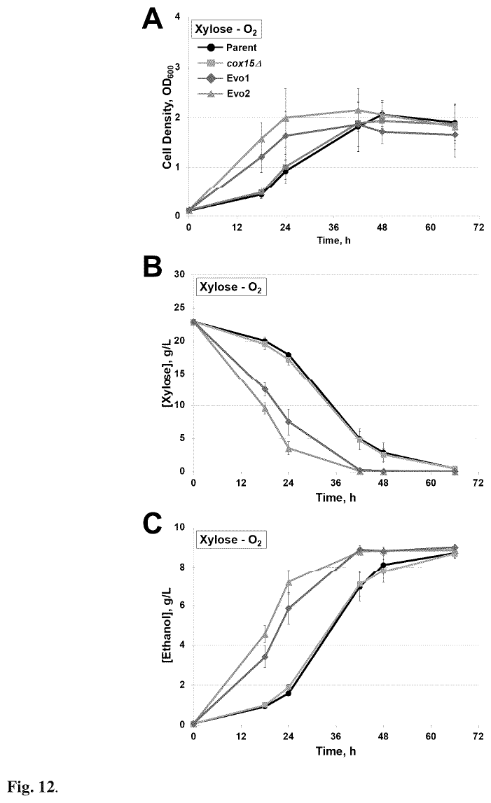

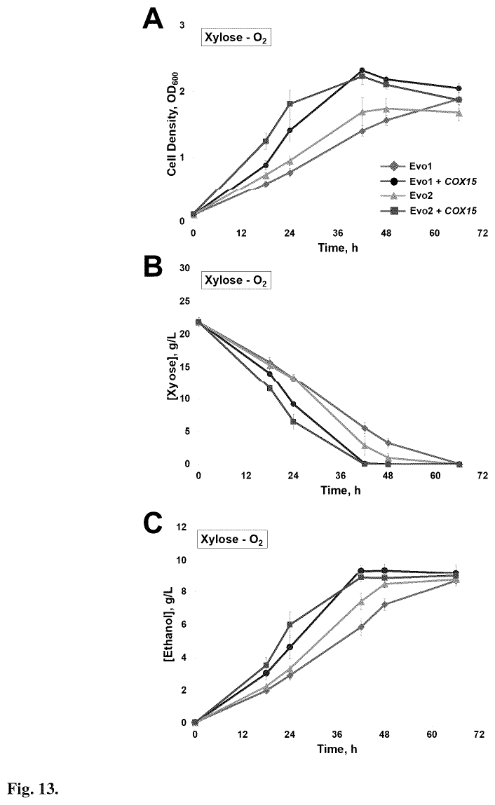

The Crabtree-Warburg Effect in S. Cerevisiae is a product of high glucose flux, which consequentially results in rapid glucose consumption aerobically and anaerobically. Thus, S. cerevisiae is used as an industrial workhorse for its ability to rapidly convert glucose into biofuels without the costly need for aeration. It was investigated if the evolved mutations positively impacted the rate of anaerobic xylose fermentation. Under anaerobic conditions, both the evolved strains fermented xylose into ethanol faster than the Parent strain, with or without COX15 (FIG. 12). Unexpectedly, the evolved strains expressing COX15 displayed slightly increased anaerobic xylose fermentation rates but had no differences in final ethanol titer (FIG. 13), suggesting that COX15 may have direct or indirect anaerobic functions in xylose fermentation. Importantly, these studies further indicate that the evolved mutations increase the rate of xylose fermentation under anaerobic conditions.

### Evolved Strains Contain Overlapping Duplications in ChrIV and XVI

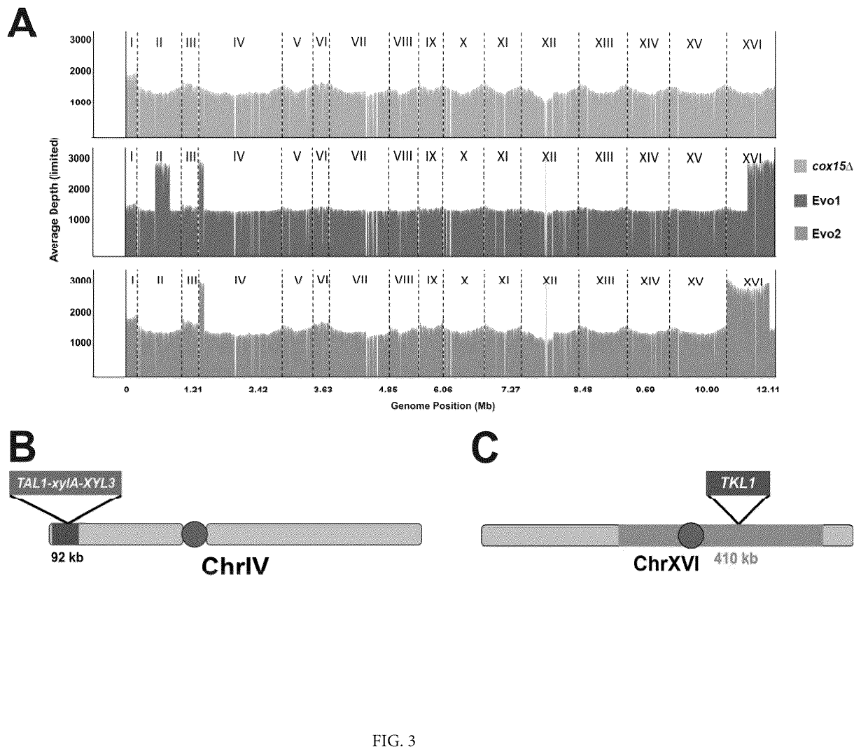

To identify the evolved genetic changes that caused the Crabtree-Warburg-like phenotype, the Evo1 and Evo2 strains were sequenced using Illumina HiSeq technology and mapped their sequencing reads to the Y22-3 parental genome (McIlwain et al., 2016). Multiple single nucleotide changes in the evolved strains were identified that were not present in the unevolved cox15Δ strain. However, the mutations in the two independently evolved strains did not overlap in the same genes or in genes within the same pathways or biological functions. Nevertheless, when comparing the read coverages across all chromosomes, it was found that the read coverages across regions of ChrII, IV, and XVI were two-fold higher in the evolved strains than the unevolved cox15Δ strain, indicating that segmental duplications occurred during the directed evolution (FIG. 3 (A)). Despite being evolved independently, both the evolved strains had duplications of nearly identical 92 kb region of ChrIV and in a large, 410 kb overlapping region of ChrXVI, whereas the ChrII duplication was only present in the Evo1 strain. It was found that the duplicated region of ChrIV contained the XYL cassette expressing TAL1, xylA, and XYL3, which had been inserted into the HO locus (Parreiras et al., 2014) (FIG. 3 (B)). The overlapping region in ChrXVI notably included TKL1, which encodes transketolase, a pentose phosphate pathway enzyme involved in xylose catabolism (FIG. 3 (C)).

### Duplications of Engineered Xylose Metabolism Genes and TKL1 are Required for Robust Aerobic Xylose Fermentation

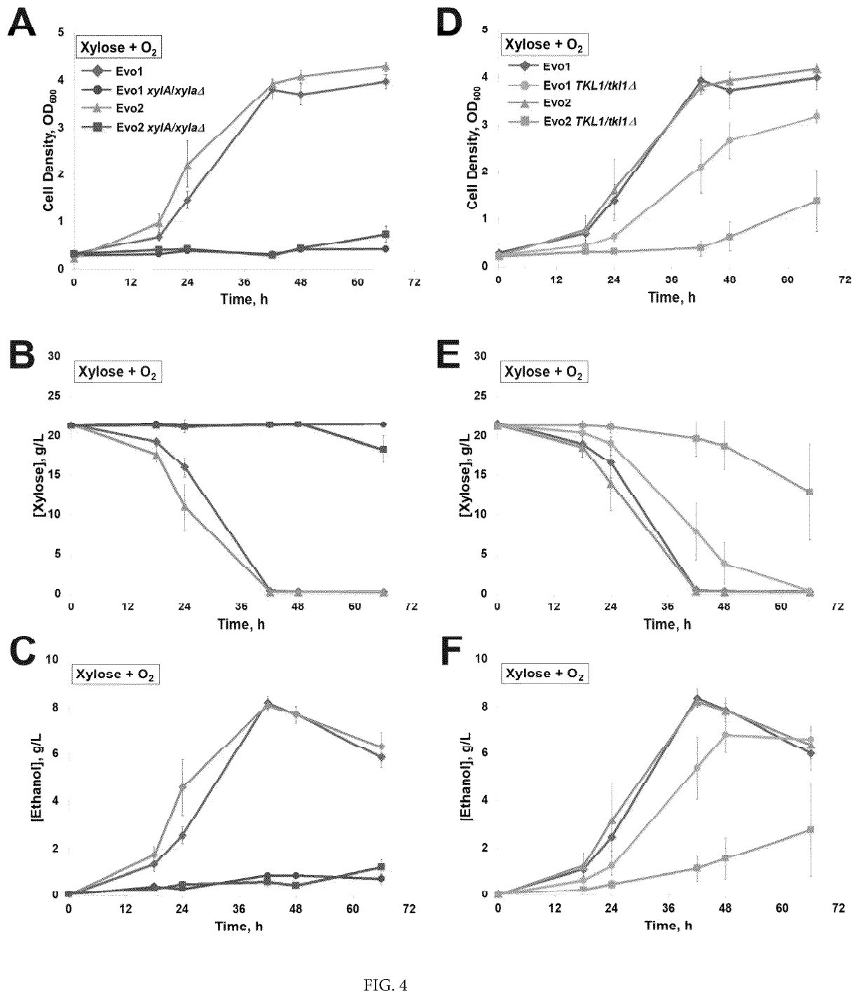

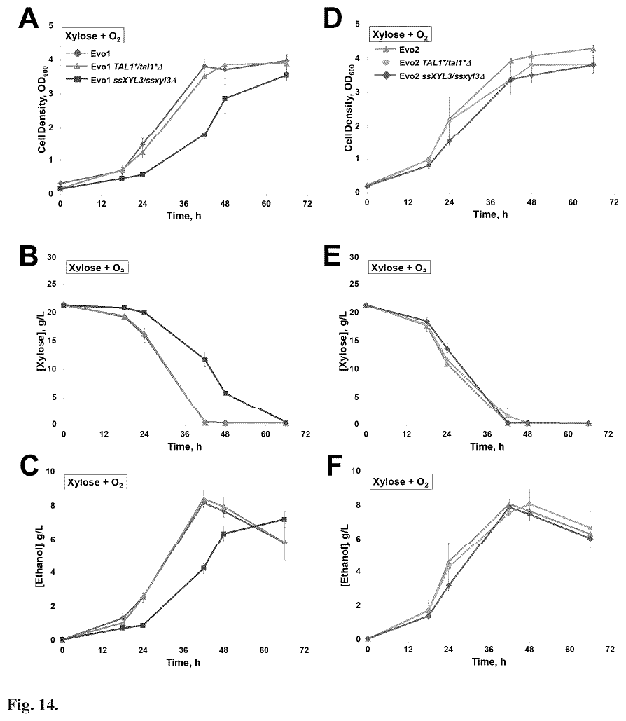

It was sought to confirm whether duplications of the engineered xylose metabolism genes TAL1, xylA, and XYL3 from ChrIV and TKL1 from ChrXVI were important for the ability of the evolved strains to ferment xylose aerobically without respiration. Each of the four genes from the evolved strains were individually deleted and tested for their requirement in aerobic xylose fermentation. Deletion of the second copy of xylA blocked aerobic xylose fermentation by both evolved strains (FIG. 4 (A-C)), whereas deletion of an extra copy of TAL1 had no effect (FIG. 14). In contrast, deletion of an extra copy of XYL3 had a modest effect on the Evo1 strain but little effect on the Evo2 strain, suggesting that the genetic bases for the Evo1 and Evo2 phenotypes are overlapping but not identical (FIG. 14). Consistent with this hypothesis, deletion of an extra copy of TKL1 had a stronger effect on the Evo2 strain, significantly decreasing aerobic xylose fermentation rate, whereas the Evo1 strain with the extra copy of TKL1 deleted displayed modestly decreased xylose fermentation (FIG. 4 (D-F). Together, these results suggest that duplications of xylA, XYL3, and TKL1 are important for aerobic xylose fermentation without respiration in the evolved strains.

### Duplicated Genes Synergize With hog1Δ, isu1Δ, and ira2Δ Mutations to Enhance Xylose Conversion Aerobically and Anaerobically

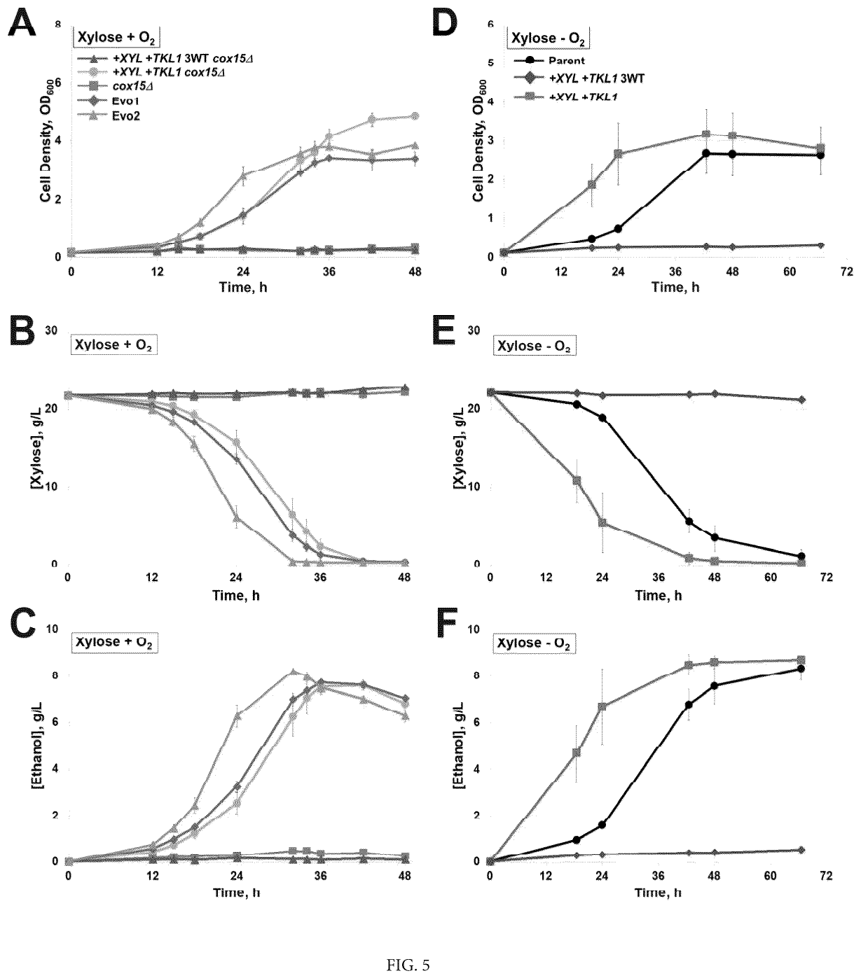

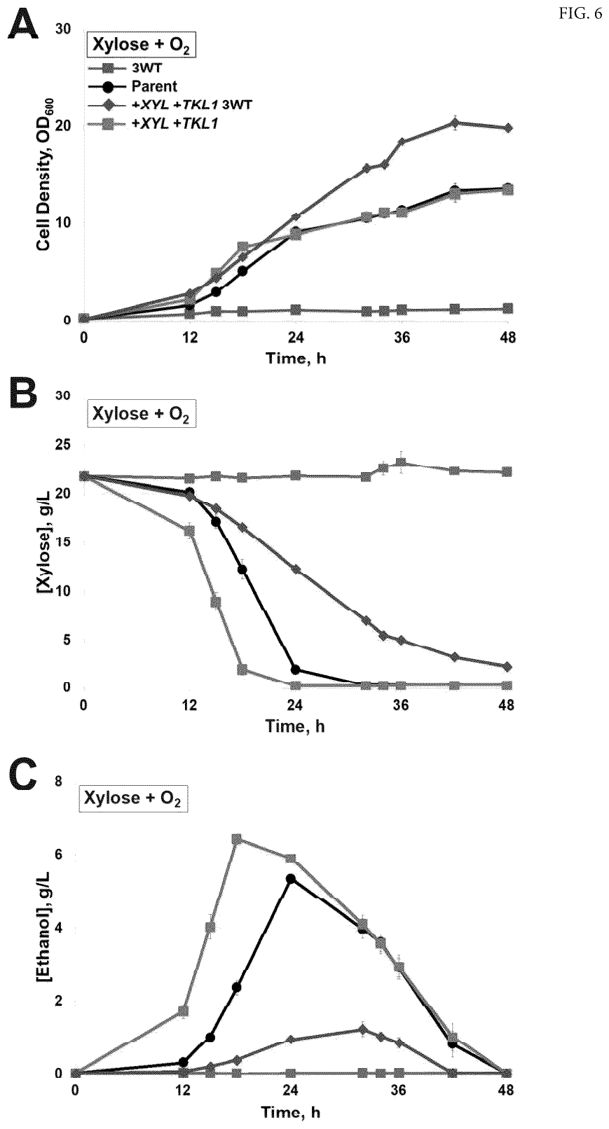

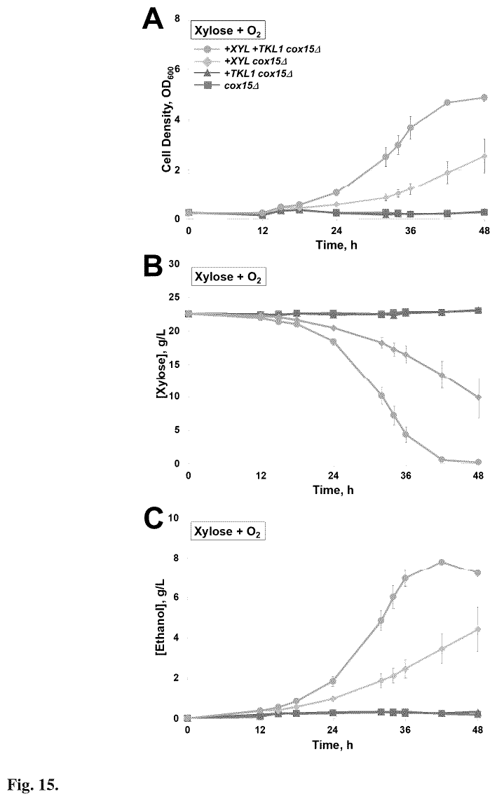

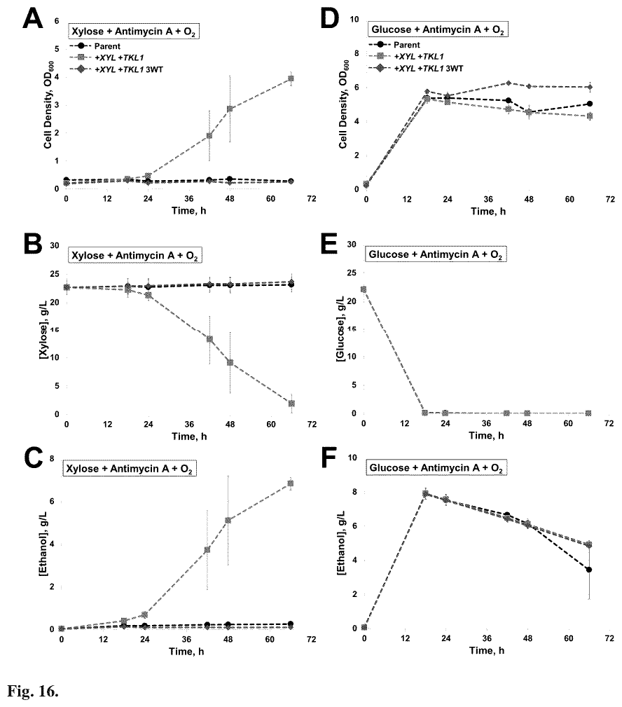

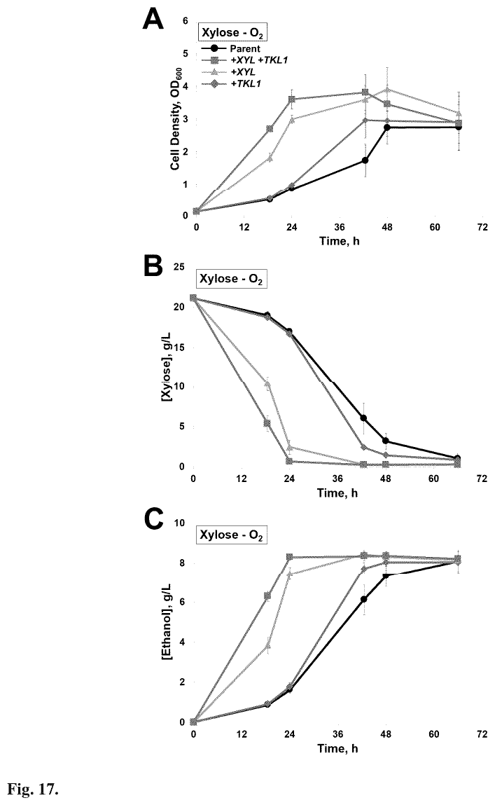

To directly test whether additional copies of the xylose metabolism genes and TKL1 enable xylose fermentation aerobically without respiration, second copies of the XYL cassette and TKL1 were engineered into the Parent strain containing the cox15Δ mutation. The cox15Δ strain engineered with additional copies of the xylose metabolism genes and TKL1 displayed rapid consumption of and growth on xylose aerobically (FIG. 5 (A-C)), similarly to the Evo1 and Evo2 strains. In contrast, insertion of an additional copy of TKL1 into the cox15Δ strain did not enable growth on xylose aerobically, while insertion of a second copy of the XYL cassette caused modestly faster growth aerobically on xylose (FIG. 15). This result suggests that duplications of the XYL cassette and TKL1 together allow for respiration-independent aerobic xylose fermentation. When two copies of the XYL cassette and TKL1 were engineered into the cox15Δ strain containing wild-type HOG1, ISU1, and IRA2 (denoted as “3WT”), the strain did not metabolize xylose aerobically (FIG. 5 (A-C)). This result further suggests that the hog1Δ, isu1Δ, and ira2Δ mutations, in combination with two copies of the XYL cassette and TKL1, enable aerobic xylose fermentation. It was shown that the increased rates of xylose conversion into ethanol by the evolved yeast strains were not dependent upon the deletion of COX15 aerobically or anaerobically (FIGS. 12 and 13). Similarly, it was found that expressing second copies of the XYL cassette and TKL1 in the COX15 -containing Parent strain also increased the rates of xylose consumption and ethanol production aerobically (FIG. 6) and increased overall xylose fermentation anaerobically (FIG. 5 (D-F)). Interestingly, when the Parent strain was engineered with a duplicated XYL cassette and TKL1, as well as wild-type HOG1, ISU1, and IRA2, it grew rapidly on xylose aerobically but produced very low amounts of ethanol, suggesting that this strain primarily respired xylose (FIG. 6). Indeed, aerobic growth and consumption of xylose, but not glucose, by this strain were completely blocked in the presence of Antimycin A. In contrast, the Parent strain engineered with the duplicated XYL cassette and TKL1, and containing deletion mutations in HOG1, ISU1, and IRA2 maintained rapid xylose conversion into ethanol aerobically under Antimycin A treatment (FIG. 16). Together, these results suggest that additional copies of the XYL cassette and TKL1 enhance aerobic respiration of xylose, while deletions of HOG1, ISU1, and IRA2 redirect xylose metabolism from respiration to fermentation. The Parent strain engineered with both the additional XYL cassette and the additional copy of TKL1, along with wild-type HOG1, ISU1, and IRA2 did not grow on xylose anaerobically (FIG. 5 (D-F)). This result further supports the conclusion that two copies of the xylose metabolism genes and TKL1 synergize with mutations in HOG1, ISU1, GRE3, and IRA2 to promote both aerobic and anaerobic xylose fermentation. When the Parent strain was engineered with an additional copy of TKL1 or the XYL cassette alone, more modest increases in the rate of xylose conversion into ethanol anaerobically were observed (FIG. 17).

### Engineered Gene Duplications and Deletion Mutations Have Broad Utility

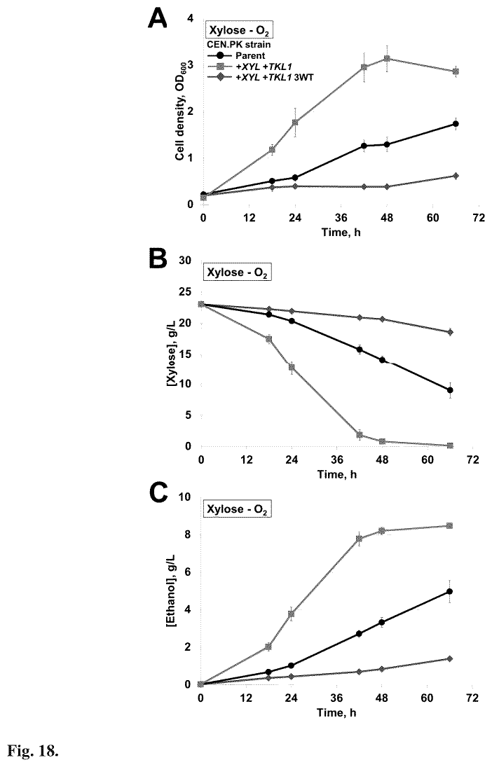

Mutations in HOG1 and IRA2/PKA pathway genes, as well as ISU1, have been shown to enhance xylose metabolism in multiple strain backgrounds (Dos Santos et al., 2016; Osiro et al., 2019; Sato et al., 2016), suggesting that these genetic modifications have universal utility. Similarly, the generality of the disclosed genetic modifications in another strain background was tested. First, additional copies of the XYL cassette and TKL1 were introduced into the commonly used strain CEN.PK113-5D (Landi et al., 2011). As in the Y22-3 strain background, extra copies of the XYL, cassette and TKL1 significantly enhanced anaerobic xylose fermentation in the CEN.PK113-5D background also containing mutations in HOG1, ISU1, GRE3, and IRA2 (FIG. 18). However, these genetic duplications were not effective in the CEN.PK113-5D background containing wild-type HOG1, ISU1, and IRA2. These results suggest that the genetic modifications disclosed herein can be applied to different strain backgrounds.

### Engineering the Duplications and Deletion Mutations Improves the Rate of Xylose Fermentation in Industrially Relevant Switchgrass Hydrolysate

As another test of the utility of the genetic modifications, it was investigated whether the combination of gene duplications and deletions enables accelerated xylose fermentation from pretreated and hydrolyzed lignocellulosic biomass to ethanol. Specifically, a comparison of the Parent strain containing single copies of the XYL cassette and TKL1, as well as deletion mutations in HOG1, ISU1, GRE3, and IRA2; the engineered Parent strain with extra copies of the XYL cassette and TKL1; and the modified Parent strain containing duplications of the XYL cassette and TKL1, but containing wild-type HOG1, ISU1, and IRA2, was conducted. The three strains were cultured anaerobically in bioreactors containing AFEX-pretreated switchgrass hydrolysate (ASGH) (Tables 1 and 2), which contains inhibitory compounds that are known to inhibit cell growth and fermentation (Parreiras et al., 2014). As when cultured anaerobically in YPX media (FIG. 5 (D-F)), the engineered Parent strain containing extra copies of the XYL cassette and TKL1 consumed xylose and produced ethanol significantly faster than the Parent strain, whereas the ethanol yield from xylose was not significantly different between these two strains (Table 1). The modified Parent strain containing the duplications but wild-type HOG1, ISU1, and IRA2 consumed little xylose and did not produce ethanol from xylose. These results suggest that extra copies of the XYL cassette and TKL1, in combination with deletion mutations in HOG1, ISU1, and IRA2, enable accelerated xylose conversion from lignocellulosic hydrolysate into ethanol anaerobically. On the other hand, duplications of the XYL cassette and TKL1 did not affect glucose conversion kinetics, whereas deletion mutations in HOG1, ISU1, and IRA2 slightly improved glucose consumption and ethanol production rates (Table 3). Thus, the genetic modifications could improve industrial processes by significantly and specifically improving the conversion of xylose into biofuels from lignocellulosic biomass.

TABLE 1
Xylose conversion kinetics of engineered S. cerevisiae strains from
AFEX-pretreated switchgrass hydrolysate (ASGH) fermentations.

| | Y1348 | | |
|---|---|---|---|
| | (+XYL, | | Y1327 |
| | +TKL1, | Y560 | (+XYL, |
| Strain | 3WT) | (4Δ) | +TKL1, 4Δ) |
| Specific xylose | 0.010 ± 0.008* | 0.048 ± 0.008** | 0.098 ± 0.004 |
| consumption rate¹ | | | |
| Specific ethanol | ND | 0.013 ± 0.002*** | 0.028 ± 0.004 |
| production rate | | | |
| from xylose² | | | |
| Ethanol productivity | ND | 0.064 ± 0.011** | 0.16 ± 0.01 |
| from xylose³ | | | |
| % of theoretical yield | ND | 43.9 ± 8.2 | 55.9 ± 7.8 |
| from xylose ⁴ | | | |

¹In g xylose consumed/OD₆₀₀/h ± SEM
²In g ethanol produced/OD₆₀₀/h ± SEM
³In g ethanol produced/L/h ± SEM
⁴ Percentage of maximum theoretical ethanol yield ± SEM
ND = Not determined; the rate of ethanol production did not exceed the rate of stripping
3WT = HOG1 ISU1 IRA2
4Δ = gre3Δ hog1Δ isu1Δ ira2Δ
*p < 0.05, Y1348 vs. Y1327
**p < 0.05, Y560 vs. Y1327
***p < 0.07, Y560 vs. Y1327

All the experiments were performed in biological triplicate.

TABLE 2
Saccharomyces cerevisiae strains used in this study.

| Strain Name | | | |
|---|---|---|---|
| (GLBRCY#) | Name in Figure | Description | Reference |
| Y22-3 | | NRRL YB-210MATa | Parreiras |
| | | spore HOΔ::ScTAL1-CpXylA- | et al. |
| | | SsXYL3-LoxP-KanMX-LoxP | (2014) |
| Y36 | 3WT | Y22-3 with LoxP-KanMX- | Parreiras |
| | | LoxP maker excised by Cre | et al. |
| | | | (2014) |
| Y286 | | Y36 gre3Δ::LoxP ira2Δ::LoxP | Sato |
| | | hog1Δ::LoxP-KanMX-LoxP | et al. |
| | | isu1Δ::LoxP | (2016) |
| Y560 | Parent | Y286 with LoxP-KanMX- | This |
| | | LoxP maker excised by Cre | study |
| Y583 | cox15Δ | Y286 cox15Δ::LoxP-HphMX-LoxP | This |
| | | | study |
| Y1031 | Evo1 | Y583 from aerobic xylose ALE | This |
| | | flask B and without KanMX | study |
| | | and HphMX cassettes | |
| Y1033 | Evo2 | Y583 from aerobic xylose ALE | This |
| | | flask A and without KanMX | study |
| | | and HphMX cassettes | |
| Y1031 + Empty | Evo1 + Empty | Y1031 transformed with | This |
| | | pRSCENHygMX plasmid | study |
| Y1033 + Empty | Evo2 + Empty | Y1033 transformed with | This |
| | | pRSCENHygMX plasmid | study |
| Y1031 + COX15 | Evo1 + COX15 | Y1031 transformed with | This |
| | | pRSCENHygMX-COX15 plasmid | study |
| Y1033 + COX15 | Evo2 + COX15 | Y1033 transformed with | This |
| | | pRSCENHygMX-COX15 plasmid | study |
| Y1176 | Evo1 xylA/xylaΔ | Evol ScTAL1-CpXylA-SsXYL3- | This |
| | | LoxP/TAL1-xylaΔ::LoxP-KanMX- | study |
| | | LoxP-SsXYL3 | |
| Y1183 | Evo2 xylA/xylaΔ | Evo2 ScTAL1-CpXylA-SsXYL3- | This |
| | | LoxP/TAL1-xylaΔ::LoxP-KanMX- | study |
| | | LoxP-SsXYL3 | |
| Y1185 | Evo1 TKL1/tkl1Δ | Evo1 TKL1/tkl1Δ::LoxP-KanMX- | This |
| | | LoxP | study |
| Y1189 | Evo2 TKL1/tkl1Δ | Evo2 TKL1/tkl1Δ::LoxP-KanMX- | This |
| | | LoxP | study |
| Y1327 | +XYL +TKL1 | Y560 gre3Δ::ScTAL1-CpXylA- | This |
| | | SsXYL3 ChrI::ScTKL1 | study |
| Y1348 | +XYL +TKL1 3WT | Y560 gre3Δ::ScTAL1-CpXylA- | This |
| | | SsXYL3 ChrI::ScTKL1 with wild- | study |
| | | type HOG1, ISU1 and IRA2 | |
| Y1374 | +XYL +TKL1 cox15Δ | Y1327 cox15Δ::LoxP-KanMX-LoxP | This |
| | | | study |
| Y1376 | +XYL +TKL1 3WT | Y1348 cox15Δ::LoxP-KanMX-LoxP | This |
| | cox15Δ | | study |

TABLE 3
Glucose conversion kinetics of engineered S. cerevisiae strains from
ASGH fermentations.

| | Y1348 | | Y1327 |
|---|---|---|---|
| | (+XYL, | | (+XYL, |
| | +TKL1, | Y560 | +TKL1, |
| Strain | 3WT) | (4Δ) | 4Δ) |
| Exponential growth rate¹ | 0.19 ± 0.01* | 0.13 ± 0.01 | 0.13 ± 0.01 |
| Specific glucose | 1.36 ± 0.10* | 1.86 ± 0.13 | 1.84 ± 0.06 |
| consumption rate² | | | |
| Specific ethanol production | 0.58 ± 0.03* | 0.82 ± 0.06 | 0.85 ± 0.02 |
| rate from glucose³ | | | |
| Ethanol productivity from | 2.27 ± 0.60 | 1.75 ± 0.20** | 1.61 ± 0.16 |
| glucose⁴ | | | |
| % of theoretical yield from | 90.3 ± 2.3 | 88.5 ± 1.8 | 89.4 ± 2.4 |
| glucose⁵ | | | |

¹In OD₆₀₀/h ± SEM
²In g glucose consumed/OD₆₀₀/h ± SEM
³In g ethanol produced/OD₆₀₀/h ± SEM
⁴In g ethanol produced/L/h ± SEM
⁵Percentage of maximum theoretical ethanol yield ± SEM
3WT = HOG1 ISU1 IRA2
4Δ = gre3Δ hog1Δ isu1Δ ira2Δ
*p < 0.05, Y1348 vs. Y1327
**p < 0.06, Y560 vs. Y1327

All the experiments were performed in biological triplicate.

TABLE 4
Xylose conversion kinetics of engineered S. cerevisiae strains from
AFEX-pretreated switchgrass hydrolysate (ASGH) fermentations.

| | Y1348 | | |
|---|---|---|---|
| | (+XYL | Y560 | Y1327 |
| Strain | +TKL1 3WT) | (Parent) | (+XYL +TKL1) |
| Specific xylose | 0.010 ± 0.008^{e} | 0.048 ± 0.008^{f} | 0.098 ± 0.004 |
| consumption | | | |
| rate^{a} | | | |
| Specific ethanol | ND | 0.013 ± 0.002^{g} | 0.028 ± 0.004 |
| production rate | | | |
| from xylose^{b} | | | |
| Ethanol | ND | 0.064 ± 0.011^{f} | 0.16 ± 0.01 |
| productivity | | | |
| from xylose^{c} | | | |
| % of theoretical | ND | 43.9 ± 8.2 | 55.9 ± 7.8 |
| yield from | | | |
| xylose ^{d} | | | |

ND = Not determined; the rate of ethanol production did not exceed the rate of stripping.
3WT = HOG1 ISU1 IRA2.
^{a}In g xylose consumed/OD₆₀₀/h ± SEM.
^{b}In g ethanol produced/OD₆₀₀/h ± SEM.
^{c}In g ethanol produced/L/h ± SEM.
^{d} Percentage of maximum theoretical ethanol yield ± SEM.
^{e}p < 0.05, Y1348 vs. Y1327.
^{f}p < 0.05, Y560 vs. Y1327.
^{g}p < 0.07, Y560 vs. Y1327.

### Discussion

Xylose has been targeted as a prospective carbon source for producing sustainable biofuels since it is the second most abundant sugar in plant-derived biomass (Lee et al., 2021). Thus, many studies have attempted to engineer S. cerevisiae to efficiently convert xylose into biofuels in a manner comparable to easily fermentable sugars like glucose (Demeke et al., 2015; Dos Santos et al., 2016; Feng et al., 2018; Lee et al., 2020; Parreiras et al., 2014; Sato et al., 2016; Zeng et al., 2017). The Crabtree/Warburg Effect was first described in tumor cells where lactate was aerobically fermented in high levels of glucose, along with decreased respiration (Crabtree, 1929; Warburg et al., 1927). This phenomenon also occurs in S. cerevisiae, which ferments high levels of glucose into ethanol under aerobic conditions (Pfeiffer and Morley, 2014). A similar effect has not been observed with xylose and other carbon sources in yeasts because it requires high metabolic flux, which is achieved for glucose partially through the repression of other metabolic pathways and the upregulation of hexose transporters and glycolytic enzymes (Kayikci and Nielsen, 2015; Pfeiffer and Morley, 2014). Through ALE and genetic manipulations, it was determined that a suite of genetic changes enable strains to grow on xylose aerobically without respiration (FIG. 3). These genetic changes impart a trait that is analogous to the Crabtree-Warburg Effect for xylose, although repression by glucosamine (FIG. 11) indicates that the aerobic fermentation of xylose and glucose are still using different metabolic and regulatory pathways. Increased expression of metabolic enzymes by integrating multiple gene copies has been shown by others to increase the rate of xylose utilization and anaerobic fermentation. In particular, several studies engineered S. cerevisiae with genes overexpressing xylose isomerase (xylA), xylulokinase (XYL3), and non-oxidative pentose phosphate pathway (RKI1, RPE1, TAL1 , TKL1, TKL2, and NQM1) in combination with the deletion of the gene encoding aldose reductase, GRE3 (Bracher et al., 2019; Demeke et al., 2013; Kuyper et al., 2005; Verhoeven et al., 2017). Deletions of several genes involved in iron-sulfur cluster biogenesis (ISU1) and various signaling pathways (HOG1, IRA2, BCY1, and SSK1) can also contribute to improved xylose metabolic flux in strains engineered with xylose isomerase, likely by reprogramming regulatory systems (Dos Santos et al., 2016; Myers et al., 2019; Sato et al., 2016; Wagner et al., 2019). Herein, it is disclosed that increased copy number of the XYL cassette and TKL1 played roles enabling aerobic xylose fermentation in a strain containing deletion mutations in HOG1, ISU1, and IRA2 (FIGS. 5 (A-C) and FIG. 6), a trait not previously described in any yeast strain. Adding only a second copy of the XYL, cassette resulted in increased xylose fermentation rate, whereas adding only a second copy of TKL1 had no effect on xylose fermentation (FIG. 15). Thus, in addition to the regulatory mutations, at least two copies of xylA allow for enzymatic capacity to initiate xylose fermentation aerobically, while a second copy of TKL1 further enhances this trait. Moreover, two copies of both the XYL cassette and TKL1 led to increased xylose fermentation rate anaerobically in lab medium and switchgrass hydrolysate (FIG. 5 (D-F), FIG. 17, and Table 1), demonstrating that the genetic changes also increase xylose metabolic flux in industrially relevant conditions.

The precise mechanism by which the mutations in HOG1, ISU1, and IRA2 synergize to enable xylose fermentation has been unclear, but recent studies suggest that these mutations alter how S. ceervisiae sense xylose. Osiro and colleagues determined that the responses of three major sugar-signaling pathways, including cAMP/PKA, Snf3p/Rgt2p, and Snf1p/Mig1p, by wild-type strains cultured in high xylose concentrations were similar to the signals induced by low glucose concentrations (Osiro et al., 2018), which typically promote respiration under aerobic conditions. In contrast, the authors later demonstrated that isu1Δira2Δ double mutants cultured in high xylose concentrations displayed activation of some high-glucose signaling pathways, suggesting that these mutations may promote the recognition of xylose as a fermentable sugar (Osiro et al., 2019). In support of this view, it was shown herein that deletions of HOG1, ISU1, and IRA2 caused the up-regulation of both the PKA and Snf1 pathways on xylose (Myers et al., 2019). PKA is normally activated in response to high glucose concentrations to promote growth and glucose fermentation, while Snf1 is normally activated when non-preferred carbon sources are consumed; this unusual regulatory combination seems to promote the fermentation of xylose with rapid uptake and high metabolic flux (Myers et al., 2019). Sugar uptake and growth rates also correlate with the hypothesized regulation of overflow metabolism by sensing glycolytic flux (Huberts et al., 2012; Litsios et al., 2018). When glycolytic flux surpasses a specific point of respiratory capacity, metabolism switches from respiration to fermentation due to the overflow of pyruvate flux to ethanol and other fermentation products (Dashko et al., 2014; Hagman and Piskur, 2015; Niebel et al., 2019). Thus, deletions of HOG1, ISU1, and IRA2 together may cause S. cerevisiae to initiate similar, but not identical, metabolic activities with xylose.

As shown herein, increasing xylose conversion in this primed regulatory background resulted in overflow metabolism of xylose into ethanol. Specifically, loss-of-function mutations in HOG1, ISU1, and IRA2 significantly increased xylose consumption and ethanol production rates aerobically in strains containing either one or two copies of the XYL cassette and TKL1 (FIG. 6), whereas the strain containing two copies of the XYL cassette and TKL1 with wild-type HOG1, ISU1, and IRA2 mostly respired xylose aerobically (FIG. 6) and did not grow on xylose anaerobically (FIG. 5 (D-F)). These results indicate that overexpression of xylose isomerase and the pentose phosphate pathway enzymes increase total metabolic conversion, while the altered regulatory pathways prime yeast to ferment xylose. Collectively, these genetic changes increase the flux of xylose into ethanol by enabling a form of xylose overflow metabolism that is analogous to how the Crabtree-Warburg Effect enables glucose fermentation under aerobic conditions (FIG. 16). Importantly, this increased metabolism also translates into faster rates of xylose fermentation in more industrially relevant anaerobic conditions.

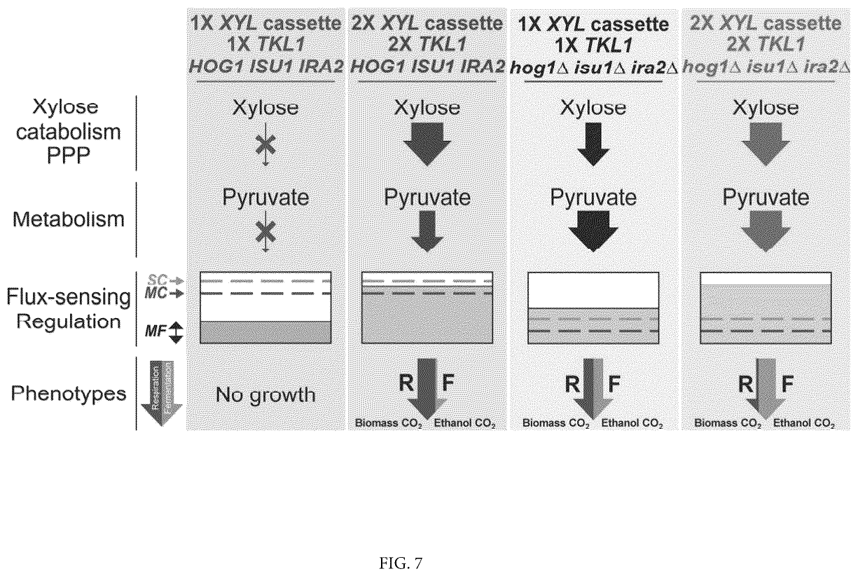

To synthesize the present observations with published work, a model of aerobic, Crabtree-Warburg-like metabolic flux for xylose is proposed (FIG. 7). It is assumed that xylose-fermenting strains have a minimal capacity (MC) for enabling aerobic growth through respiration and a minimal switching capacity (SC) for changing from respiratory to fermentative flux. Once total metabolic flux exceeds the MC value, a strain can utilize xylose through respiration. (FIG. 7, second column). Regulatory pathways altered in hog1Δisu1Δira2Δ triple mutants lower both MC and SC, causing the total metabolic flux (MF) of the strain to exceed both capacities. As xylose consumption rates and MF level increase beyond this lowered SC level, yeast begin to switch metabolic flux from respiration into fermentation. For yeast containing both the deletion mutations and the duplicated copies of the XYL cassette and TKL1 (FIG. 7, right-most green column), the increased metabolic flux far exceeds the switching capacity, resulting in much greater conversion of xylose into ethanol aerobically.

### Conclusion

Disclosed herein are defined genetic modifications that enable multiple xylose isomerase-expressing S. cerevisiae strains to successfully convert xylose from lab media and switchgrass hydrolysate into ethanol both aerobically and anaerobically. The theoretical yield of ethanol from xylose was still lower than that from glucose. However, altering tolerance to lignocellulose-derived inhibitors, to improve xylose uptake, and to derepress genes involved in xylose metabolism may improve the yield. Furthermore, the evolved strains have slightly different aneuploidies, and the Evo2 strain is more efficient for aerobic and anaerobic xylose fermentation than the Evo1 strain, suggesting that there are additional unknown mutations that may improve xylose conversion. Nonetheless, the yeast strains described here are able to ferment xylose anaerobically, and also preferentially fermenting xylose in the presence of oxygen, a hallmark of the Crabtree-Warburg Effect previously only seen in yeast during the aerobic fermentation of glucose.

### REFERENCES

Bamba et al., AMB Express. 6:4 (2016).
Barros et al., FEBS Lett., 492:133 (2001).
Bracher et al., FEMS Yeast Res., 19:______ (2019).
Brat et al., Appl. Environ. Microbiol., 75:2304 (2009).
Conant & Wolfe, PLoS Biol., 4:e109 (2006).
Crabtree, Biochem J., 23:536 (1929).
Cunha et al., Biotechnol. Biofuels, 12:20 (2019).
Dashko et al., Fems. Yeast Res., 14:826 (2014).
Demeke et al., Biotechnol. Biofuels, 6:89 (2013).
Demeke et al., Plos Genetics, 11:______ (2015).
Dos Santos et al., Sci. Rep., 6:38676 (2016).
Feng et al., PLoS One, 13:e0195633 (2018).
Gietz et al., Nat. Protoc., 2:31 (2007).
Glerum et al., J. Biol. Chem., 272:19088 (1997).
Guldener et al., Nucleic Acids Res., 24:2519 (1996).
Hagman & Piskur, PLoS One, 10:e0116942 (2015).
Hahn-Hagerdal et al., Appl. Microbiol. Biotechnol., 74:937 (2007).
Higgins et al., Genetics, 210:219 (2018).
Hittinger & Carroll, Nature, 449:677 (2007).
Huberts et al., Fems. Yeast Res., 12:118 (2012).
Jeong et al., PLoS One, 15:e0236294 (2020).
Jin et al., Appl. Environ. Microbiol., 70:6816 (2004).
Jin et al., J. Microbiol. Biotechn., 10:564 (2000).
Johansson et al., Appl. Environ. Microb., 67:4249 (2001).
Johnston & Kim, Biochem. Soc. T., 33:247 (2005).
Kayikci & Nielsen, FEMS Yeast Res., 15:______ (2015).
Kim et al., Biotechnol. Adv., 31:851 (2013).
Kotter & Ciriacy, Appl. Microbiol. Biot., 38:776 (1993).
Kuyper et al., FEMS Yeast Res., 5:399 (2005).
Kwak & Jin, Microb. Cell Fact, 16:82 (2017).
Landi et al., Biochem. Biophys. Res. Commun., 414:604 (2011).
Langdon et al., Mol. Biol. Evol., 35:2835 (2018).
Lee et al., Curr. Opin. Biotechnol., 67:15 (2021).
Lee et al., Biotechnol. Biofuels, 13:5 (2020).
Lee et al., Appl. Environ. Microbiol., 78:5708 (2012).
Lee et al., Biotechnol. Biofuels, 7:122 (2014).
Li, Aligning sequence reads, clone sequences and assembly contigs with BWA-MEM. arXiv preprint arXiv: 1303.3997 (2013).
Lin & Li, Mol. Biol. Evol., 28:131 (2011).
Litsios et al., Curr. Opin. Microbiol., 42:71 (2018).
Magtanong et al., Nat. Biotechnol., 29:505 (2011).
McIlwain et al., G3-Genes Genom. Genet., 6:1757 (2016).
Mckenna et al., Genome Res., 20:1297 (2010).
Myers et al., PLoS Genet., 15:e1008037 (2019).
Narayanaswamy et al., Bioresour. Technol., 102:6995 (2011).
Niebel et al., Nat. Metab., 1:125 (2019).
Osiro et al., Microb. Cell. Fact., 18:88 (2019).
Osiro et al., FEMS Yeast Res., 18:______ (2018).
Parreiras et al., PLoS One, 9:e107499 (2014).
Pauly & Keegstra, Plant J., 54:559 (2008).
Pfeiffer & Morley, Front. Mol. Biosci., 1:17 (2014).
Pronk et al., Yeast, 12:1607 (1996).
Reider Apel et al., Sci. Rep., 6:19512 (2016).
Runquist et al., Microb. Cell. Fact., 8:49 (2009).
Salusjarvi et al., Microb. Cell. Fact., 7:18 (2008).
Sato et al., PLoS Genet., 12:e1006372 (2016).
Scalcinati et al., FEMS Yeast Res., 12:582 (2012).
Schwalbach et al., Appl. Environ. Microbiol., 78:3442 (2012).
Sherman, Methods Enzymol., 350:3 (2002).
Stoneman et al., G3 (Bethesda), 10:4287 (2020).
Sun & Jin, Biotechnol. J., 16:e2000142 (2021).
Thompson et al., Elife, 2:e00603 (2013).
Traff et al., Appl. Environ. Microbiol., 67:5668 (2001).
Verhoeven et al., Sci. Rep., 7:46155 (2017).
Wagner et al., PLoS One, 14:e0212389 (2019).
Warburg et al., J. Gen. Physiol., 8:519 (1927).
Williams et al., Front Energy Res., 6:______ (2019).
Yamanaka, Arch. Biochem. Biophys., 131:502 (1969).
Zeng et al., Appl. Microbiol. Biotechnol., 101:1753 (2017).
Zhang et al., Bioresource Tech. Rep., 11:100517 (2020).
   All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1-30. (canceled)

31. A recombinant yeast comprising:
one or more recombinant nucleic acids that recombinantly express one or more of:
a xylulokinase;
a xylose isomerase;
a transaldolase; and
a transketolase; and
one or more disabling mutations of or in one or more of:
a Hog1 gene;
an Isu1 gene;
an Ira1 or Ira2 gene; and
a Gre3 gene.

32. The recombinant yeast of claim 31, wherein the recombinant yeast comprises one or more disabling mutations of or in one or more of:
a Hog1 gene;
an Isu1 gene; and
an Ira1 or Ira2 gene.

33. The recombinant yeast of claim 32, wherein the one or more recombinant nucleic acids recombinantly express each of:
a xylulokinase;
a xylose isomerase; and
a transketolase.

34. The recombinant yeast of claim 33, wherein the recombinant yeast comprises one or more disabling mutations of or in each of:
a Hog1 gene;
an Isu1 gene; and
an Ira1 or Ira2 gene.

35. The recombinant yeast of claim 33, wherein the recombinant yeast comprises one or more disabling mutations of or in each of:
a Hog1 gene;
an Isu1 gene;
an Ira1 or Ira2 gene; and
a Gre3 gene.

36. The recombinant yeast of claim 32, wherein the one or more recombinant nucleic acids recombinantly express each of:
a xylulokinase;
a xylose isomerase;
a transaldolase; and
a transketolase.

37. The recombinant yeast of claim 36, wherein the recombinant yeast comprises one or more disabling mutations of or in each of:
a Hog1 gene;
an Isu1 gene; and
an Ira1 or Ira2 gene.

38. The recombinant yeast of claim 36, wherein the recombinant yeast comprises one or more disabling mutations of or in each of:
a Hog1 gene;
an Isu1 gene;
an Ira1 or Ira2 gene; and
a Gre3 gene.

39. The recombinant yeast of claim 31, wherein the one or more recombinant nucleic acids recombinantly express each of:
a xylulokinase;
a xylose isomerase;
a transaldolase; and
a transketolase.

40. The recombinant yeast of claim 31, wherein the one or more recombinant nucleic acids comprise any combination of:
at least two genes that recombinantly express a xylulokinase;
at least two genes that recombinantly express a xylose isomerase; and
at least two genes that recombinantly express a transketolase.

41. The recombinant yeast of claim 31, wherein the one or more recombinant nucleic acids comprise at least two genes that recombinantly express a xylose isomerase.

42. The recombinant yeast of claim 41, wherein the recombinant yeast comprises one or more disabling mutations of or in each of:
a Hog1 gene;
an Isu1 gene; and
an Ira1 or Ira2 gene.

43. The recombinant yeast of claim 31, wherein the one or more recombinant nucleic acids comprise each of:
at least two genes that recombinantly express a xylulokinase;
at least two genes that recombinantly express a xylose isomerase;
at least one gene that recombinantly expresses a transaldolase; and
at least two genes that recombinantly express a transketolase.

44. The recombinant yeast of claim 43, wherein the recombinant yeast comprises one or more disabling mutations of or in each of:
a Hog1 gene;
an Isu1 gene; and
an Ira1 or Ira2 gene.

45. The recombinant yeast of claim 44, wherein the recombinant yeast exhibits aerobic xylose fermentation.

46. The recombinant yeast of claim 43, wherein the recombinant yeast comprises one or more disabling mutations of or in each of:
a Hog1 gene;
an Isu1 gene;
an Ira1 or Ira2 gene; and
a Gre3 gene.

47. The recombinant yeast of claim 46, wherein the recombinant yeast exhibits aerobic xylose fermentation.

48. The recombinant yeast of claim 31, wherein the recombinant yeast exhibits aerobic xylose fermentation.

49. A method of fermenting xylose comprising contacting the xylose with the recombinant yeast of claim 31 to thereby ferment the xylose.

50. The method of claim 49, wherein the fermenting the xylose converts the xylose into ethanol.


SEQUENCE LISTING (20 sequences)

SEQ ID NO: 1 - DNA, 20 bp, synthetic construct
  source 1..20: mol_type = other DNA
atgaaggagaaaactcaccg

SEQ ID NO: 2 - protein, 3079 aa, synthetic construct
MSQPTKNKKKEHGTDSKSSRMTRTLVNHILFERILPILPVESNLSTYSEVEEYSSFISCRSVLINVTVSRDANAMVEGTLELIESLLQGHEIISDKGSSDVIESILIILRLLSDALEYNWQNQESLHYNDISTHVEHDQEQKYRPKLNSILPDYSSTHSNGNKHFFHQSKPQALIPELASKLLESCAKLKFNTRTLQILQNMISHVHGNILTTLSSSILPRHKSYLTRHNHPSHCKMIDSTLGHILRFVAASNPSEYFEFIRKSVQVPVTQTHTHSHSHSHSLPSSVYNSIVPHFDLFSFIYLSKHNFKKYLELIKNLSVTLRKTIYHCLLLHYSAKAIMFWIMARPAEYYELFNLLKDNNNEHSKSLNTLNHTLFEEIHSTFNVNSMITTNQNAHQGSSSPSSSSPSSPPSSSSSDNNNQNIIAKSLSRQLSHHQSYIQQQSERKLHSSWTTNSQSSTSLSSSTSNSTTTDFSTHTQPGEYDPSLPDTPTMSNITISASSLLSQTPTPTTQLQQRLNSAAAAAAAAASPSNSTPTGYTAEQQSRASYDAHKTGHTGKDYDEHFLSVTRLDNVLELYTHFDDTEVLPHTSVLKFLTTLTMFDIDLFNELNATSFKYIPDCTMHRPKERTSSFNNTAHETGSEKTSGIKHITQGLKKLTSLPSSTKKTVKFVKMLLRNLNGNQAVSDVALLDTMRALLSFFTMTSAVFLVDRNLPSVLFAKRLIPIMGTNLSVGQDWNSKINNSLMVCLKKNSTTFVQLQLIFFSSAIQFDHELLLARLSIDTMANNLNMQKLCLYTEGFRIFFDIPSKKELRKAIAVKISKFFKTLFSIIADILLQEFPYFDEQITDIVASILDGTIINEYGTKKHFKGSSPSLCSTTRSRSGSTSQSSMTPVSPLGLDTDICPMNTLSLVGSSTSRNSDNVNSLNSSPKNLSSDPYLSHLVAPRARHALGGPSSIIRNKIPTTLTSPPGTEKSSPVQRPQTESISATPMAITNSTPLSSAAFGIRSPLQKIRTRRYSDESLGKFMKSTNNYIQEHLIPKDLNEATLQDARRIMINIFSIFKRPNSYFIIPHNINSNLQWVSQDFRNIMKPIFVAIVSPDVDLQNTAQSFMDTLLSNVITYGESDENISIEGYHLLCSYTVTLFAMGLFDLKINNEKRQILLDITVKFMKVRSHLAGIAEASHHMEYISDSEKLTFPLIMGTVGRALFVSLYSSQQKIEKTLKIAYTEYLSAINFHERNIDDADKTWVHNIEFVEAMCHDNYTTSGSIAFQRRTRNNILRFATIPNAILLDSMRMIYKKWHTYTHSKSLEKQERNDFRNFAGILASLSGILFINKKILQEMYPYLLDTVSELKKNIDSFISKQCQWLNYPDLLTRENSRDILSVELHPLSFNLLFNNLRLKLKELACSDLSIPENESSYVLLEQIIKMLRTILGRDDDNYVMMLFSTEIVDLIDLLTDEIKKIPAYCPKYLKAIIQMTKMFSALQHSEVNLGVKNHFHVKNKWLRQITDWFQVSIAREYDFENLSKPLKEMDLVKRDMDILYIDTAIEASTAIAYLTRHTFLEIPPAASDPELSRSRSVIFGFYFNILMKGLEKSSDRDNYPVFLRHKMSVLNDNVILSLTNLSNTNVDASLQFTLPMGYSGNRNIRNAFLEVFINIVTNYRTYTAKTDLGKLEAADKFLRYTIEHPQLSSFGAAVCPASDIDAYAAGLINAFETRNATHIVVAQLIKNEIEKSSRPTDILRRNSCATRSLSMLARSKGNEYLIRTLQPLLKKIIQNRDFFEIEKLKPEDSDAERQIELFVKYMNELLESISNSVSYFPPPLFYICQNIYKVACEKFPDHAIIAAGSFVFLRFFCPALVSPDSENIIDISHLSEKRTFISLAKVIQNIANGSENFSRWPALCSQKDFLKECSDRIFRFLAELCRTDRTIDIQVRTDPTPIAFDYQFLHSFVYLYGLEVRRNVLNEAKHDDGDIDGDDFYKTTFLLIDDVLGQLGQPKMEFSNEIPIYIREHMDDYPELYEFMNRHAFRNIETSTAYSPSVHESTSSEGIPIITLTMSNFSDRHVDIDTVAYKFLQIYARIWTTKHCLIIDCTEFDEGGLDMRKFISLVMGLLPEVAPKNCIGCYYFNVNETFMDNYGKCLDKDNVYVSSKIPHYFINSNSDEGLMKSVGITGQGLKVLQDIRVSLHDITLYDEKRNRFTPVSLKIGDIYFQVLHETPRQYKIRDMGTLFDVKFNDVYEISRIFEVHVSSITGVAAEFTVTFQDERRLIFSSPKYLEIVKMFYYAQIRLESEYEMDNNSSTSSPNSNNKDKQQKERTKLLCHLLLVSLIGLFDESKKMKNSSYNLIAATEASFGLNFGSHFHRSPEVYVPEDTTTFLGVIGKSLAESNPELTAYMFIYVLEALKNNVIPHVYIPHTICGLSYWIPNLYQHVYLADDEEGPENISHIFRILIRLSVRETDFKAVYMQYVWLLLLDDGRLTDIIVDEVINHALERDSENRDWKKTISLLTVLPTTEVANNIIQKILAKIRSFLPSLKLEAMTQSWSELTILVKISIHVFFETSLLVQMYLPEILFIVSLLIDVGPRELRSSLHQLLMNVCHSLAINSALPQDHRNNLDEISDIFAHQKVKFMFGFSEDKGRILQIFSASSFASKFNILDFFINNILLLMEYSSTYEANVWKTRYKKYVLESVFTSNSFLSARSIMIVGIMGKSYITEGLCKAMLIETMKVIAEPKITDEHLFLAISHIFTYSKIVEGLDPNLDLMKHLFWFSTLFLESRHPIIFEGALLFVSNCIRRLYMAQFENESETSLISTLLKGRKFAHTFLSKIENLSGIVWNEDNFTHILIFIINKGLSNPFIKSTAFDFLKMMFRNSYFEHQINQKSDHYLCYMFLLYFVLNCNQFEELLGDVDFEGEMVNIENKNTIPKILLEWLSSDNENANITLYQGAILFKCSVTDEPSRFRFALIIRHLLTKKPICALRFYSVIRNEIRKISAFEQNSDCVPLAFDILNLLVTHSESNSLEKLHEESIERLTKRGLSIVTSSGIFAKNSDMMIPLDVKPEDIYERKRIMTMILSRMSCSA

SEQ ID NO: 3 - protein, 165 aa, synthetic construct [sequence omitted]

SEQ ID NO: 4 - protein, 327 aa, synthetic construct
MSSLVTLNNGLKMPLVGLGCWKIDKKVCANQIYEAIKLGYRLFDGACDYGNEKEVGEGIRKAISEGLVSRKDIFVVSKLWNNFHHPDHVKLALKKTLSDMGLDYLDLYYIHFPIAFKYVPFEEKYPPGFYTGADDEKKGHITEAHVPIIDTYRALEECVDEGLIKSIGVSNFQGSLIQDLLRGCRIKPVALQIEHHPYLTQEHLVEFCKLHDIQVVAYSSFGPQSFIEMDLQLAKTTPTLFENDVIKKVSQNHPGSTTSQVLLRWATQRGIAVIPKSSKKERLLGNLEIEKKFTLTEQELKDISALNANIRFNDPWTWLDGKFPTFA

SEQ ID NO: 5 - protein, 435 aa, synthetic construct
MTTNEEFIRTQIFGTVFEITNRYNDLNPVGMGAFGLVCSATDTLTSQPVAIKKIMKPFSTAVLAKRTYRELKLLKHLRHENLICLQDIFLSPLEDIYFVTELQGTDLHRLLQTRPLEKQFVQYFLYQILRGLKYVHSAGVIHRDLKPSNILINENCDLKICDFGLARIQDPQMTGYVSTRYYRAPEIMLTWQKYDVEVDIWSAGCIFAEMIEGKPLFPGKDHVHQFSIITDLLGSPPKDVINTICSENTLKFVTSLPHRDPIPFSERFKTVEPDAVDLLEKMLVFDPKKRITAADALAHPYSAPYHDPTDEPVADAKFDWHFNDADLPVDTWRVMMYSEILDFHKIGGSDGQIDISATFDDQVAAATAAAAQAQAQAQAQVQLNMAAHSHNGAGTTGNDHSDIAGGNKVSDHVAANDTITDYGNQAIQYANEFQQ

SEQ ID NO: 6 - protein, 486 aa, synthetic construct
MLFRNIEVGRQAAKLLTRTSSRLAWQSIGASRNISTIRQQIRKTQLYNFKKTVSIRPFSLSSPVFKPHVASESNPIESRLKTSKNVAYWLIGTSGLVFGIVVLGGLTRLTESGLSITEWKPVTGTLPPMNQKEWEEEFIKYKESPEFKLLNSHIDLDEFKFIFFMEWIHRLWGRAIGAVFILPAVYFAVSKKTSGHVNKRLFGLAGLLGLQGFVGWWMVKSGLDQEQLDARKSKPTVSQYRLTTHLGTAFFLYMGMLWTGLEILRECKWIKNPVQAISLFKKLDNPAIGPMRKISLALLAVSFLTAMSGGMVAGLDAGWVYNTWPKMGERWFPSSRELMDENFCRREDKKDLWWRNLLENPVTVQLVHRTCAYVAFTSVLAAHMYAIKKKAVIPRNAMTSLHVMMGVVTLQATLGILTILYLVPISLASIHQAGALALLTSSLVFASQLRKPRAPMRNVIITLPHSSKVTSGKILSEASKLASKPL

SEQ ID NO: 7 - DNA, 1308 bp, synthetic construct
  source 1..1308: mol_type = other DNA
atgaccactaacgaggaattcattaggacacagatattcggtacagttttcgagatcacaaatagatacaatgatttaaaccccgttgggatgggggcatttgggttggtttgctcagccacggacactttgacatctcagccagttgccattaagaaaatcatgaaacctttttccactgcagtgctggccaaaaggacatatcgtgaactaaaactactaaaacatctaagacacgagaacttgatttgccttcaggacatatttctttctccattggaagatatatattttgtcacggaattacaaggaacagatttacatagactcttgcaaacaagacccttggaaaagcaatttgttcagtatttcctataccaaattctaaggggtttaaaatacgttcactccgcgggcgtcattcatagagatttgaaaccgagcaacattctgattaatgaaaactgtgatttgaagatttgcgatttcggtctagcaagaattcaagaccctcaaatgacaggctatgtttccactagatactacagggcacctgaaatcatgctaacgtggcaaaaatatgacgtcgaggtcgacatttggtccgctggttgtatttttgccgaaatgattgaaggtaagcctttgttccctgggaaagatcatgttcaccaattttcgatcatcactgacttgttgggatctccgccaaaggatgtgataaatactatttgttccgaaaatactctaaaatttgttacttcgttaccacacagagatccaattccattttctgaaagatttaaaacagtcgaacctgatgccgtagaccttttggaaaaaatgctggtttttgatcctaagaagagaatcactgcggcggatgccttggctcatccttattcggctccttaccacgatccaacggatgaaccagtagccgatgccaagttcgattggcactttaatgacgctgatctgcctgtcgatacctggcgtgttatgatgtactcagaaatcctagacttccataagattggtggcagtgatggacagattgatatatctgccacgtttgatgaccaagttgctgcagccaccgctgccgcggcgcaggcacaggctcaggctcaggctcaagttcagttaaacatggctgcgcattcgcataatggcgctggcactactggaaatgatcactcagatatagctggtggaaacaaagtcagcgatcatgtagctgcaaatgacaccattacggactacggtaaccaggccatacagtacgctaatgagttccaacagtaa

SEQ ID NO: 8 - DNA, 9632 bp, synthetic construct
  source 1..9632: mol_type = other DNA
cacccgtcctgtggatcatcttttgccctgcaaatagagcttcaaacttaacattcttcttcagcatataacatacaacaagattaaggctctttctaaaatgaatcaaagcgatccgcaagacaaaaaaaatttcccaatggaatactctttgaccaagcatctttttttttgataggcttctacttgttcttcccatagaatctaatttgaaaacatatgctgatgtggaggcagattcagttttcaattcgtgtcggtccatcattttgaatatagccatcactaaggacttgaacccgattatcgaaaacacattaggtttaattgacttgattgtgcaagatgaagaaattacgtctgacaatattacagatgatattgcccattctatattggttcttttgagattactgagtgatgtttttgagtattactgggatcaaaacaatgacttcaagaaaattagaaacgataattacaaaccgggattttcaagtcacaggccaaacttccatacatctaggccaaagcacacgagaatcaatccagctttggcgacgatgttactatgtaaaatttctaagctgaagttcaatacaagaactttaaaggttttacagaacatgtctcaccatctttctggcagcgctactatctcaaaatcgagtattttacccgattcacaggaatttttacaaaagagaaactatccagcatataccgagaaaatagatttaacaatagattatatccagagattcatatctgcttccaatcatgttgaattcacaaagtgtgtcaaaacaaaagttgttgcacctttattgatatcacacacctcaaccgaattgggcgtagtaaaccacttggatttatttggttgtgagtatttgactgataagaatctgctagcatatctggacatactacaacacctgtcaagttacatgaagaggaccatttttcattcgcttttgttatattatgcttccaaagcttttttattttggataatggcaaggccaaaagaatacgtcaaaatttataacaatctaatatcatcagattataatagtccgtcttcttcatctgataatggtggttcgaataattctgataaaacgtctatatcccaactagtctcactgttattcgatgacgtttattccacttttagtggatcatcattattaacaaatgtcaataatgaccaccactaccatcttcatcattcatcttcttcatcaaagacgaccaacactaatagtccaaactctatatcaaaaacgtcgataaagcagtcgagtgtgaatgcttctggcaatgtttctccgtctcagttttctactgggaatgatgcatcgcctacttcccctatggcatcattgagttcacccttaaacacgaacatcctagggtatccgttatctccaatcacttcaacactaggacaggcgaatacttccacatcgactacggctgcaactaccaaaacagatgcagatacgccctctactatgaatactaacaacaataataacaataacaacagcgctaatcttaataatattccacaacgcatattttccttagatgacatttcatcctttaactcgagtagaaaatcactcaatctagatgatagtaactccttgtttctttgggatacttctcagcattctaatgcatcgatgacaaatacaaatatgcatgcaggagttaataattctcagtctcagaacgatcagtcttctttaaactatatggaaaatattatggagctgtattccaactataccggatcagaactatcctcccatactgccatattaaggtttttggtggttctgaccttattagacagtgaagtatatgatgagatgaactcaaattcgtatagaaaaatttcggaaccgataatgaatattaatccgaaggactctaatacttcaagttggggctcagcatccaaaaacccaagtatcaggcacctcacccatggcttaaaaaaacttactttacagcaaggcaggaaacgtaacgttaaatttttgacatatttgattagaaatttgaatgggggacagttcgtttcagatgtttccttgattgactctatcaggtccattctattcttaatgacaatgacgtcttctatatcccaaatcgattcaaatattgcttctgttattttttcgaagagattctacaacttgttgggtcaaaatttagaggtcggcaccaattggaattctgccactgcaaatacttttatttctcattgtgttgaaaggaatccccttacacataggcgtttacaattagagttttttgcaagcggtttacagctggattctgatttatttttaagacatttacaactggaaaaagaactcaatcacatagaccttcccaaaatatcgttatacactgaaggatttagggtattttttcacctagtaagcaccaaaaaacttcatgaggatattgcagaaaaaacctcctctgtgttaaagagacttttctgcataattgctgatattttgttgaaagcaacgccttattttgacgataatgtaaccaagattattgcttctatattggatgggcatattttagatcaatttgacgctgcgcgaacactttctaatgatgatcatgtcagttttgatgctgccacaagcgtttacactgagccaaccgaaattattcataactcatcggatgcctcgttagtctcttcactttcccaatcacccttatcaattaactcaggaagcaatatcaccaatacgcgcacctgggatattcaatcaatcttaccaaccttatcgaacagatcaagtgcttctgatttgagcttgtctaacattttgactaatccgttggaggcacaacaaaataataatgcaaacttgttagcccatagattatctggggttcctactactaagagatacgcttcaccgaacgactctgaaagatcacgacaaagtccatattcttctccgccgcaattgcaacaaagtgacttgccttctccgctctcagtcctctcgtcaagtgcaggattttcttctaatcattcgattacggcaaccccaactattttgaaaaacatcaaatctccaaaaccaaacaaaacaaaaaaaattgctgatgataaacaattgaaacagccttcttattcaagagtaatactgagtgacaatgatgaagcaagaaagattatgatgaacattttcagcattttcaaaagaatgaccaactggtttatacgcccagatgctaatacagaattcccgaagacttttacggatattataaaaccactttttgtctctatattggattctaatcaaagactacaagttacagcgcgggcttttattgaaatcccattaagttatatagctacttttgaagacattgataatgatcttgacccaagagtactgaatgaccattatttgttatgtacatatgccgttactttgtttgcttcatcattgtttgatttgaagttagaaaatgcgaagagagagatgctactagacattattgttaaatttcaacgagtccgttcttatttatcaaatttagcagaaaaacacaacctagtccaggcaataattacgacggagaggttgacgctgccattattagttggtgctgtaggaagtggaattttcatttcattatactgcagtcgtggaaatacgccacgcttaataaaaatttcatgttgtgaatttctacgatccttgagattttatcaaaaatacgtaggcgctttggatcaatattccatttacaatattgatttcatagatgctatggcccaggacaatttcactgcctcaggatcagtggctttgcaacggcgtctaagaaataatattttaacttatatcaaaggatccgactcaatccttttggattcaatggacgtgatttacaagaagtggttttacttcagctgttcgaaatcagttacgcaagaggaactagtagattttagaagcttggcaggcattctagcttctatgtcaggtattctgtctgatatgcaagagttggaaaaaagcaagagcgctccagataatgaaggagacagcttatcatttgagtcacgaaatcccgcttatgaggtgcacaaaagtcttaaactcgagttaacgaaaaaaatgaatttctttatttcaaaacaatgtcaatggttgaataatccaaatctattaacaagagaaaattcgagagatatattaagtattgagttgcatcctctatcttttaacttattgtttaacaacctaggactgaaaatagatgaactgatgtcaattgatctttcaaagtcacatgaggattcatcgtttgttttactagagcagataataattataataagaactatactaaagagggatgatgatgaaaagataatgctactcttttcgacggacttgcttgatgcggtcgataagttgatcgaaatagtggagaaaatttccatcaagtcctccaaatattataagggaattatccaaatgtcgaaaatgtttagagcatttgagcactctgaaaagaacctgggcatttcaaatcatttccatttaaagaataaatggttgaagttagttattggttggttcaaactatctattaataaggattatgattttgaaaacctgtcaagaccattaagggaaatggatttgcagaaaagggacgaagattttttgtatatcgacacttctattgaatctgcaaaagcattggcttacctaacacataatgttcctttagaaataccgccttcaagctcaaaagaagattggaacagatcttctacagtatcatttggcaatcactttactattttgttaaaaggtctggagaaaagcgcggacctgaatcagtttccagtttcattaaggcataagatcagtatacttaatgaaaatgtaataatagcgctaacgaacttatctaatgccaatgtcaacgtttctttaaaattcactttaccaatgggttattctccaaataaagatatcagaatcgcctttttaagagttttcatcgacatagtaaccaactatccagttaaccctgagaaacatgaaatggataaaatgctagctatagacgacttcctgaaatatataatcaagaacccaatattagcatttttcggaagtttagcgtgttctcctgctgatgttgatttatatgctggtggattcttaaacgcctttgacactagaaatgcgtctcatatccttgttactgagctccttaaacaagaaatcaaacgggccgcaagatcagacgatattctcagaagaaatagttgtgcaacaagggctttgtcactttacactagatctagaggtaacaaatatttgataaaaactttgagacccgttttgcaagggatagtggataacaaggagtcttttgaaattgataagatgaaaccaggatccgaaaactccgaaaagatgttagacttatttgagaagtacatgacaagattaattgacgcaattacaagttctattgatgatttcccaatagaattagttgatatctgtaaaacaatttacaatgctgctagtgtaaattttccagaatacgcatatattgccgttgggtcattcgttttcttgaggtttatcgggcctgctttagttagtcctgattcggaaaatatcattattgttacgcacgcccatgacagaaagccctttattacactagctaaagttattcaaagtttagctaatggcagggaaaatatattcaagaaagatatcttagtttcaaaagaagagtttttgaaaacctgtagtgataaaatattcaattttttgtctgaattgtgcaagataccgactaacaatttcaccgtcaatgtaagagaagatccgacaccaataagctttgactactcatttttgcataaattcttttacctcaatgagtttaccataagaaaagaaattattaatgaatccaaattaccaggggagttcagctttttgaaaaatactgttatgctcaacgacaaaattcttggtgtattgggacaacctagcatggaaataaaaaatgaaattcctccttttgtagtcgagaatcgggaaaaatatccttcattgtatgaattcatgagtcgctatgccttcaaaaaagtggacatgaaagaagaagaagaggataatgcgccatttgtacatgaagcaatgacattggatggcatacaaatcattgtcgtaacttttaccaattgcgagtacaataattttgtaatggactcactggtctataaagttctgcagatatatgcaagaatgtggtgctctaaacattatgtagttatcgattgtaccaccttttatgggggtaaggctaatttccaaaaattgactactctatttttcagtttgataccagagcaagcatcaagtaattgtatgggatgttattacttcaacgtcaacaaatcatttatggaccaatgggcctcatcatatactgtagaaaatccgtacttggtcactacaattccccgttgtttcatcaacagcaatactgaccaaagtttgataaagtccttaggattgagtggtaggagtttggaagttttgaaagatgtaagagttactttgcatgatattaccctttatgacaaggaaaaaaagaagttttgtcccgtgtccttgaagataggaaacaaatacttccaagttttacatgagattccgcagttgtacaaggttaccgtatcaaacaggacattcagcatcaaattcaacaatgtttacaagatatcaaatttaatttcagtcgatgtctctaacaccacaggcgtttcctcggaatttacgttaagtcttgataatgaagaaaagttggtattttgcagtccgaagtacctagaaattgtgaaaatgttttattatgcccagttaaagatggaagaagactttggtacggatttttcgaacgatatttcattttcaacatcctcttcagcagttaatgcttcttactgcaatgttaaagaagttggtgaaattatatcacatttgtcattggtgatccttgtaggtttattcaatgaggatgatctcgtcaaaaacatatcatacaaccttctcgtggcaacgcaagaagcatttaatttagattttgggacaaggcttcacaaatccccagagacatatgtacccgatgataccaccacgttcttggccctaattttcaaggctttttcagaatcttcaacggaactaactccatatatatggaaatatatgctggatggccttgaaaacgacgtgattcctcaagaacatattcctacggttgtctgttcattgtcatactgggtaccaaacttatatgaacatgtatatttggcaaatgacgaagagggaccagaggcgatttcacgtataatctatagcttaatcaggttgacggtcaaagagccaaatttcacgacagcttaccttcaacagatttggtttttactggcattggatggtcgtctcacgaacgtgatagttgaagaaatagtaagtcatgcgctggatagagattcagaaaacagagactggatgaaagctgtgtcaatactaaccagttttccaacgacagagattgcttgtcaggtaatagagaagctaataaatatgatcaaatcttttctaccttctctagcagttgaggcttccgcacacagttggtctgagcttactattttatcaaaaattagtgtgtcaattttctttgaatcacccttactttcccagatgtatttaccggagattcttttcgctgtgtctctgttaattgatgtcggtccttcggaaataagagtctcattgtacgagttgttgatgaatgtttgtcattctttaaccaacaatgagtccttacctgaaaggaataggaaaaatttggatatcgtctgtgcaacattcgcacgtcaaaagttgaactttatctccggttttagccaagaaaaaggtagagttttaccaaattttgccgcttcctccttctccagtaaattcggaacattagatctcttcactaaaaacattatgctattgatggaatatggttctatttcagagggtgcacaatgggaggcaaaatataagaaatatttgatggatgcgatttttggccatcggtcgttcttctctgcgagagctatgatgattctaggtataatgagtaagtcgcacacgtcccttttcctttgtaaagaacttttagttgaaaccatgaaggtcttcgcagagccagttgtggatgatgaacaaatgttcatcattatagctcatgtctttacttacagcaaaattgtcgaagggttagatccttcttcagaattaatgaaagagctattttggcttgctacaatatgtgttgaatcccctcatcctttactctttgaaggtggtctcctgttcatggtaaattgtttgaagcgactgtacacggtccatcttcaacttggattcgatggcaaatcgctagccaaaaaattaatggaatctagaaattttgctgctacgcttttggctaagttagagtcatacaatggatgcatatggaacgaagataattttcctcatattattttaggtttcattgcaaacggtttatccattcctgtcgtaaaaggagccgcattagattgtctacaggcccttttcaagaatacatattacgaaagaaagtccaacccaaaatcctccgattatctttgttaccttttcttactccatttggtcttaagtcctgaacaactttctaccttgttacttgaagtcggcttcgaagatgaactggtacctttaaataatacactaaaagtgccacttactttgatcaactggctaagttcagactcagataaatctaatatagtcttataccaaggagcacttttgtttagctgtgttatgtcagacgaaccatgtaaattccgttttgctctattgatgaggtatttgctcaaagtcaaccctatttgtgtattcaggttctatacgctgactagaaaggaattcaggaggttatcaaccctagaacaatcatctgaagcggttgctgtctcttttgaattgattgggatgcttgttacacacagtgagtttaattacctagaggaatttaatgatgaaatggtcgaacttttaaaaaagagaggcttgtcagttgtgaagcctctggatatttttgatcaggaacatatagaaaagttaaaaggagagggtgaacatcaagtggcaatttatgagagaaaaagattagcaacaatgatactggcaagaatgtcgtgctcctaattttaaattagagtattgcagtgattatatacgtttttccttataattatattttgttttatatttgttggattgtagattttaaattaaatgaaacgtagagatttgtgtaaagtataatgattgcaaggttaagagtgaaacatttttagagcactactaactagcattttccaaggtggaaagatccgaaaaagcacgattaccggaaatccaactgttgaccatttccaaacttgcctccggttgatc

SEQ ID NO: 9 - skipped

SEQ ID NO: 10 - protein, 438 aa, synthetic construct
MKNYFPNVPEVKYEGPNSTNPFAFKYYDAERIVAGKTMKEHCRFALSWWHTLCAGGADPFGVTTMDRSYGNITDPMEFAKAKVDAGFELMTKLGIEYFCFHDADIAPEGENFEESKKNLFVIVDYIKEKMDQTGIKLLWGTANNFGHPRFMHGASTSCNADVFAYAAAKIKNALDATIKLGGKGYVFWGGREGYETLLNTDLGLELDNMARLMKMAVEYGRANGFDGDFYIEPKPKEPTKHQYDFDTATVLGFLRKYGLEKDFKMNIEANHATLAGHTFEHELALARVNGVFGSVDANQGDPNLGWDTDQFPTDVHSATLAMLEVLKAGGFTNGGLNFDAKVRRGSFEFDDIAYGYIAGMDTFALGLIKAAEIIEDGRIAKFVEDRYASYKTGIGKAIVDGTTSLEELEQYVLTHNEPVMQSGRQEVLESIVNNILFR

SEQ ID NO: 11 - protein, 438 aa, synthetic construct
MKNYFPNVPEVKYEGPNSTNPFAFKYYDANKVVAGKTMKEHCRFALSWWHTLCAGGADPFGVTTMDRTYGNITDPMELAKAKVDAGFELMTKLGIEFFCFHDADIAPEGDTFEESKKNLFEIVDYIKEKMDQTGIKLLWGTANNFSHPRFMHGASTSCNADVFAYAAAKIKNALDATIKLGGKGYVFWGGREGYETLLNTDLGLELDNMARLMKMAVEYGRANGFDGDFYIEPKPKEPTKHQYDFDTATVLAFLRKYGLEKDFKMNIEANHATLAGHTFEHELAMARVNGAFGSVDANQGDPNLGWDTDQFPTDVHSATLAMLEVLKAGGFTNGGLNFDAKVRRGSFEFDDIAYGYIAGMDTFALGLIKAAEIIDDGRIAKFVDDRYASYKTGIGKAIVDGTTSLEELEQYVLTHSEPVMQSGRQEVLETIVNNILFR

SEQ ID NO: 12 - protein, 438 aa, synthetic construct; identical to SEQ ID NO: 11

SEQ ID NO: 13 - protein, 623 aa, synthetic construct
MTTTPFDAPDKLFLGFDLSTQQLKIIVTDENLAALKTYNVEFDSINSSVQKGVIAINDEISKGAIISPVYMWLDALDHVFEDMKKDGFPFNKVVGISGSCQQHGSVYWSRTAEKVLSELDAESSLSSQMRSAFTFKHAPNWQDHSTGKELEEFERVIGADALADISGSRAHYRFTGLQIRKLSTRFKPEKYNRTARISLVSSFVASVLLGRITSIEEADACGMNLYDIEKREFNEELLAIAAGVHPELDGVEQDGEIYRAGINELKRKLGPVKPITYESEGDIASYFVTRYGFNPDCKIYSFTGDNLATIISLPLAPNDALISLGTSTTVLIITKNYAPSSQYHLFKHPTMPDHYMGMICYCNGSLAREKVRDEVNEKFNVEDKKSWDKFNEILDKSTDFNNKLGIYFPLGEIVPNAAAQIKRSVLNSKNEIVDVELGDKNWQPEDDVSSIVESQTLSCRLRTGPMLSKSGDSSASSSASPQPEGDGTDLHKVYQDLVKKFGDLYTDGKKQTFESLTARPNRCYYVGGASNNGSIIRKMGSILAPVNGNYKVDIPNACALGGAYKASWSYECEAKKEWIGYDQYINRLFEVSDEMNSFEVKDKWLEYANGVGMLAKMESELKH

SEQ ID NO: 14 - protein, 623 aa, synthetic construct; identical to SEQ ID NO: 13

SEQ ID NO: 15 - protein, 623 aa, synthetic construct
MTTTPFDAPDKLFLGFDLSTQQLKIIVTDENLAALKTYNVEFDSINSSVQKGVIAINDEISKGAIISPVYMWLDALDHVFEDMKKDGFPFNKVVGISGSCQQHGSVYWSRTAEKVLSELDAESSLSSQMRSAFTFKHAPNWQDHSTGKELEEFERVIGADALADISGSRAHYRFTGLQIRKLSTRFKPEKYNRTARISLVSSFVASVLLGRITSIEEADACGMNLYDIEKREFNEELLAIAAGVHPELDGVEQDGEIYRAGINELKRKLGPVKPITYESEGDIASYFVTRYGFNPDCKIYSFTGDNLATIISLPLAPNDALISLGTSTTVLIITKNYAPSSQYHLFKHPTMPDHYMGMICYCNGSLAREKVRDEVNEKFNVEDKKSWDKFNEILDKSTDFNNKLGIYFPLGEIVPNAAAQIKRSVLNSKNEIVDVELGDKNWQPEDDVSSIVESQTLSCRLRTGPMLSKSGDSSASSSASPQPEGDGTDLHKVYQDLVKKFGDLFTDGKKQTFESLTARPNRCYYVGGASNNGSIIXKMGSILAPVNGNYKVDIPNACALGGAYKASWSYECEAKKEWIGYDQYINRLFEVSDEMNSFEVKDKWLEYANGVGMLAKMESELKH

SEQ ID NO: 16 - protein, 623 aa, synthetic construct; identical to SEQ ID NO: 13

SEQ ID NO: 17 - protein, 680 aa, synthetic construct
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 18 - protein, 680 aa, synthetic construct; identical to SEQ ID NO: 17

SEQ ID NO: 19 - protein, 335 aa, synthetic construct
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 20 - protein, 335 aa, synthetic construct; identical to SEQ ID NO: 19